(19) United States
(12) Patent Application Publication
(10) Pub. No.: US 2021/0079057 A1
(43) Pub. Date: Mar. 18, 2021

(54) COMPOSITIONS AND METHODS FOR TCR REPROGRAMMING USING FUSION PROTEINS

(71) Applicant: TCR2 THERAPEUTICS INC., Cambridge, MA (US)

(72) Inventors: Patrick BAEUERLE, Gauting (DE); Gregory SIECZKIEWICZ, Cambridge, MA (US); Robert HOFMEISTER, Scituate, MA (US)

(21) Appl. No.: 16/622,791

(22) PCT Filed: Jun. 13, 2018
(86) PCT No.: PCT/US2018/037387

§ 371 Date: Dec. 13, 2019

Related U.S. Application Data
(60) Provisional application No. 62/671,333, filed on May 14, 2018.
(60) Provisional application No. 62/629,504, filed on Feb. 12, 2018.
(60) Provisional application No. 62/554,715, filed on Sep. 6, 2017.
(60) Provisional application No. 62/519,034, filed on Jun. 13, 2017.

(51) Int. Cl. C07K 14/725; C07K 16/30; A61K 35/17; A61P 35/00; A61K 45/06; A61K 38/17; A61K 39/395

(52) U.S. Cl. CPC C07K 14/7051; C07K 16/3076; A61K 35/17; A61P 35/00; A61K 45/06; A61K 2039/505; A61K 39/39558; C07K 2319/30; C07K 2319/33; C07K 2317/569; A61K 38/1774

(57) ABSTRACT

Provided herein are T cell receptor (TCR) fusion proteins (TFPs), T cells engineered to express one or more TFPs, and methods of use thereof for the treatment of diseases, including cancer.

### CROSS-REFERENCE

This application claims the benefit of U.S. Provisional Application No. 62/519,034, filed Jun. 13, 2017; U.S. Provisional Application No. 62/554,715, filed Sep. 6, 2017; U.S. Provisional Application No. 62/629,504, filed Feb. 12, 2018; and U.S. Provisional Application No. 62/671,333, filed May 14, 2018, which applications are incorporated herein by reference in its entirety.

### BACKGROUND OF THE INVENTION

Most patients with late-stage solid tumors are incurable with standard therapy. In addition, traditional treatment options often have serious side effects. Numerous attempts have been made to engage a patient's immune system for rejecting cancerous cells, an approach collectively referred to as cancer immunotherapy. However, several obstacles make it rather difficult to achieve clinical effectiveness. Although hundreds of so-called tumor antigens have been identified, these are often derived from self and thus can direct the cancer immunotherapy against healthy tissue, or are poorly immunogenic. Furthermore, cancer cells use multiple mechanisms to render themselves invisible or hostile to the initiation and propagation of an immune attack by cancer immunotherapies.

Recent developments using chimeric antigen receptor (CAR) modified autologous T cell therapy, which relies on redirecting genetically engineered T cells to a suitable cell-surface molecule on cancer cells, show promising results in harnessing the power of the immune system to treat B cell malignancies (see, e.g., Sadelain et al., Cancer Discovery 3:388-398 (2013)). The clinical results with CD-19-specific CAR-TT cells (called CTL019) have shown complete remissions in patients suffering from chronic lymphocytic leukemia (CLL) as well as in childhood acute lymphoblastic leukemia (ALL) (see, e.g., Kalos et al., Sci Transl Med 3:95ra73 (2011), Porter et al., NEJM 365:725-733 (2011), Grupp et al., NEJM 368:1509-1518 (2013)). An alternative approach is the use of T cell receptor (TCR) alpha and beta chains selected for a tumor-associated peptide antigen for genetically engineering autologous T cells. These TCR chains will form complete TCR complexes and provide the T cells with a TCR for a second defined specificity. Encouraging results were obtained with engineered autologous T cells expressing NY-ESO-1-specific TCR alpha and beta chains in patients with synovial carcinoma.

Besides the ability of genetically modified T cells expressing a CAR or a second TCR to recognize and destroy respective target cells in vitro/ex vivo, successful patient therapy with engineered T cells requires the T cells to be capable of strong activation, expansion, persistence over time, and, in case of relapsing disease, to enable a ‘memory’ response. High and manageable clinical efficacy of CAR-TT cells is currently limited to BCMA- and CD-19-positive B cell malignancies and to NY-ESO-1-peptide expressing synovial sarcoma patients expressing HLA-A2. There is a clear need to improve genetically engineered T cells to more broadly act against various human malignancies. Described herein are novel fusion proteins of TCR subunits, including CD3 epsilon, CD3gamma and CD3 delta, and of TCR alpha and TCR beta chains with binding domains specific for cell surface antigens that have the potential to overcome limitations of existing approaches. Described herein are novel fusion proteins that more efficiently kill target cells than CARs, but release comparable or lower levels of proinflammatory cytokines. These fusion proteins and methods of their use represent an advantage for T cell receptor (TCR) fusion proteins (TFPs) relative to CARs because elevated levels of these cytokines have been associated with dose-limiting toxicities for adoptive CAR-T therapies.

### SUMMARY OF THE INVENTION

Provided herein are T cell receptor (TCR) fusion proteins (TFPs), T cells engineered to express one or more TFPs, and methods of use thereof for the treatment of diseases.

In one aspect, provided herein is a method of providing an anti-tumor immunity in a mammal comprising administering to the mammal an effective amount of a population of T cells transduced with a recombinant nucleic acid molecule encoding a T cell receptor (TCR) fusion protein (TFP) comprising a TCR subunit comprising at least a portion of a TCR extracellular domain, and a TCR intracellular domain comprising a stimulatory domain from an intracellular signaling domain of CD3epsilon or CD3gamma; and a human or humanized antibody domain comprising an antigen binding domain that is an anti-mesothelin binding domain; wherein the TCR subunit and the antibody domain are operatively linked, wherein the TFP incorporates into a TCR when expressed in a T cell, and wherein lower levels of cytokines are released following treatment compared to the cytokine levels of a mammal treated with a CAR-T cell comprising the same human or humanized antibody domain.

In some embodiments, the human or humanized antibody domain is the VHH domain set forth in SEQ ID NO:53, SEQ ID NO:54, or SEQ ID NO:55.

In some embodiments, the cell is an autologous T cell.

In some embodiments, the cell is an allogeneic T cell.

In some embodiments, the mammal is a human.

In one aspect, provided herein is a method of treating a mammal having a disease associated with expression of mesothelin comprising administering to the mammal an effective amount of a population of T cells transduced with a recombinant nucleic acid molecule encoding a T cell receptor (TCR) fusion protein (TFP) comprising a TCR subunit comprising at least a portion of a TCR extracellular domain, and a TCR intracellular domain comprising a stimulatory domain from an intracellular signaling domain of CD3epsilon or CD3gamma; and a human or humanized antibody domain comprising an antigen binding domain that is an anti-mesothelin binding domain; wherein the TCR subunit and the antibody domain are operatively linked, wherein the TFP incorporates into a TCR when expressed in a T cell, and wherein lower levels of cytokines are released following treatment compared to the cytokine levels of a mammal treated with a CAR-T cell comprising the same human or humanized antibody domain.

In some embodiments, the human or humanized antibody domain is the VHH domain set forth in SEQ ID NO:53, SEQ ID NO:54, or SEQ ID NO:55.

In some embodiments, the cell is an autologous T cell.

In some embodiments, the cell is an allogeneic T cell.

In some embodiments, the disease associated with mesothelin expression is selected from the group consisting of a proliferative disease, a cancer, a malignancy, and a non-cancer related indication associated with expression of mesothelin.

In some embodiments, the disease is a cancer selected from the group consisting of mesothelioma, renal cell carcinoma, stomach cancer, breast cancer, lung cancer, ovarian cancer, prostate cancer, colon cancer, cervical cancer, brain cancer, liver cancer, pancreatic cancer, thyroid cancer, bladder cancer, ureter cancer, kidney cancer, endometrial cancer, esophogeal cancer, gastric cancer, thymic carcinoma, cholangiocarcinoma and stomach cancer.

In some embodiments, the disease is a cancer selected from the group consisting of mesothelioma, papillary serous ovarian adenocarcinoma, clear cell ovarian carcinoma, mixed Mullerian ovarian carcinoma, endometroid mucinous ovarian carcinoma, pancreatic adenocarcinoma, ductal pancreatic adenocarcinoma, uterine serous carcinoma, lung adenocarcinoma, extrahepatic bile duct carcinoma, gastric adenocarcinoma, esophageal adenocarcinoma, colorectal adenocarcinoma, breast adenocarcinoma, a disease associated with mesothelin expression, and combinations thereof.

In some embodiments, the cells expressing a TFP molecule are administered in combination with an agent that increases the efficacy of a cell expressing a TFP molecule.

In some embodiments, for a given cytokine, at least 10% less amount of the given cytokine is released following treatment compared to an amount of the given cytokine of a mammal treated with a CAR-T cell comprising the same human or humanized antibody domain.

In some embodiments, the given cytokine comprises one or more cytokines selected from the group consisting of IL-2, IFN-γ, IL-4, TNF-α, IL-6, IL-13, IL-5, IL-10, sCD137, GM-CSF, MIP-1α, MIP-1β, and any combinations thereof.

In some embodiments, a tumor growth in the mammal is inhibited such that a size of the tumor is at most 10%, at most 20%, at most 30%, at most 40%, at most 50%, or at most 60% of a size of a tumor in a mammal treated with T cells that do not express the TFP after at least 8 days of treatment, wherein the mammal treated with T cells expressing TFP and the mammal treated with T cells that do not express the TFP have the same tumor size before the treatment.

In some embodiments, the tumor growth in the mammal is completely inhibited.

In some embodiments, the tumor growth in the mammal is completely inhibited for at least 20 days, at least 30 days, at least 40 days, at least 50 days, at least 60 days, at least 70 days, at least 80 days, at least 90 days, at least 100 days, or more.

In some embodiments, the population of T cells transduced with TFP kill similar amount of tumor cells compared to the CAR-T cells comprising the same human or humanized antibody domain.

In some embodiments, the population of T cells transduced with the TFP have a different gene expression profile than the CAR-T cells comprising the same human or humanized antibody domain.

In some embodiments, an expression level of a gene is different in the T cells transduced with the TFP than an expression level of the gene in the CAR-T cells comprising the same human or humanized antibody domain.

In some embodiments, the gene has a function in antigen presentation, TCR signaling, homeostasis, metabolism, chemokine signaling, cytokine signaling, toll like receptor signaling, MMP and adhesion molecule signaling, or TNFR related signaling.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-C are a set of graphs showing CyTOF analysis of mesothelin binder SD1 TFP-T cells or SD1-BBζ CAR-T cells were thawed and cultured overnight as described above. In FIG. 1A, SD1 TFP T cells and SD1-BBζ CAR-T cells (CAR) were labelled with 36 cell surface expressed molecules (CD57, CCR10, CXCR3, CD161, CD69, CD44, CD27, CD95, CD152, CD2, CD62L, CD3, CD137, LAG3, CCR4, OX40, CD16, CD279, CD127, CD11a, CD5, CCR5, CD4, CD8A, CD28, ICOS, CD49D, CD7, TIM3, CD45RO, CD197, CD25, CD40, MH1, CD96, HLADR). Non-biased, multiparametric analysis was conducted using t-distributed stochastic neighbor embedding (tSNE), implemented in R. Data are representative of at least 3 replicates, stained using a barcoding approach (labelling CD45). SD1 TFP-T cells show a unique phenotype, characterized by lower activation molecule expression (FIG. 1B) and higher chemokine receptor expression (FIG. 1C).

FIG. 2 is a set of graphs showing ex vivo analysis of SD1 TFP T cells or SD1 CAR-T cells from MSTO^{MSLN} xenografted mice.

FIGS. 3A-I are a set of graphs demonstrating that SD1 TFP T cells can control tumor growth in MSTO^{MSLN} xenografted mice, and show an increased amount of T cells in circulation compared to mice treated with SD1 CAR-T cells. Ex vivo analysis of total CD3+ T cells (FIG. 3A) and transduced (CD3+/GFP+; FIG. 3B) T cells in the blood of NSG mice xenografted s.c. with MSTO^{MSLN} cells 56 days after inoculation showed increased numbers of circulating GFP+ SD1 TFP-T cells. A similar phenomenon was observed in total CD3+ cells (FIG. 3C) and GFP+ SD1 TFP-T cells (FIG. 3D) in the spleen. Interestingly, plasma MSLN levels (FIG. 3E) correlated with tumor volume (FIG. 3F) on day 56. Transduction efficiency of TC-210 and MH1-BBζ CAR (BBζ CAR) T cells prior to injection is shown in FIG. 3G. Tumor volumes of individual mice upon TC-210 and BBζ CAR-T cell treatment is shown in FIG. 3H. Expression of co-stimulatory molecules on MSTO^{MSLN} cells is shown in FIG. 3I.

FIGS. 4A-J are a series of graphs demonstrating that at Day 7 post-T cell injection mice have more circulating SD1 TFP T cells SD1 CAR-T cells in vivo. Mice bearing MSTO^{MSLN} tumors were treated with no T cells (n=4), non-transduced (NT) T cells (n=5), SD1 TFP T cells (n=9) or SD1-BBζ CAR-T cells (n=9). FIG. 4A shows the measurement of tumor burden on Day 6. FIG. 4B shows tissue sections of tumors from animals sacrificed on day 7. The tumors sections were stained with anti-CD3 (maroon) and anti-mesothelin (brown) antibodies, with a hematoxylin counter stain. The top left panel shows a section of tumor from animals treated with no T cells, top right with non-transduced (NT) T cells, bottom left with SD1 TFP T cells, and bottom right with SD1 CAR-T cells. The amount of either circulating T cells from blood (FIGS. 4C-F) or spleen (FIGS. 4G-J). Using flow cytometry, total circulating T cells (CD3+; FIG. 4C) and SD1 TFP T cells (CD3+GFP+; FIG. 4D) as well as their phenotype (FIG. 4E) and activation (FIG. 4F) status was examined. This was compared to the total T cells (FIG. 4G) and SD1 TFP T cell number (FIG. 4H), and their phenotype (FIG. 4I) and activation (FIG. 4J) profile in the spleen. In FIGS. 4E-F and 4I-J, for each x-axis label the dta are represented pairwise with dots representing SD1 TFP T cells on the left and dots representing SD1 CAR-T cells on the right.

FIGS. 5A-B are a series of graphs demonstrating that SD1 TFP T cells release lower levels of cytokines than SD1 CAR-T cells in vivo. The Day 7 post-treatment plasma of the animals described in FIGS. 4A-J was analyzed for levels of cytokines (FIG. 5A) and cytotoxic payload proteins (FIG. 5B). Analytes were measured using the human CD8+ T cell Magnetic Bead Panel (Millipore). *, p<0.05; **, p<0.01; ***, p<0.001; ****, p<0.0001. NT, non-transduced T cells. In each panel, the x-axis represents, from left to right, plasma from untreated mice, mice treated with nontransduced T cells (NT), Mice treated with SD1 TFP T cells, and mice treated with SD1 CAR-T cells.

FIG. 6A is a schematic of differential expression of TruC variants.

FIG. 6B is a set of graphs showing activated T cells transduced with lentivirus encoding TRuCs and expanded in the presence of IL-2. The transduction efficiency and surface expression level of the TRuCs was detected by MSLN_Fc binding using flow cytometry. As used herein, “TRuC” represents “TFP”.

FIGS. 7A-C demonstrate that TRuC-T cells outperform CAR-T cells in a mesothelioma model. FIG. 7A shows a graph of in vitro MSTO^{MSLN}-luciferase tumor cell lysis by T cells in vitro. T cells and target cells were co-cultured for 24 hours at a ratio of 1:1. Tumor cell lysis was measured as the luciferase activity of residual alive cells. FIG. 7B is a schematic of TC210 treatment of NSG mouse MSTOMSLN mesothelioma model. FIG. 7C is a series of graphs of tumor volume based estimation of tumor clearance over time. Ctrl, tumor alone.

FIGS. 8A-C demonstrate that TC-210 T cells persist and control MSTO^{MSLN} tumors upon re-challenge. FIG. 8A is a schematic of a MSTO^{MSLN} mesothelioma model testing efficacy of TC-210. FIG. 8B is a schematic CD4/CD8 T cell ratio and transduction efficiency of TC-210 after in vitro expansion. FIG. 8C is a schematic showing the average tumor volumes measured by caliper.

FIGS. 9A-B show that ovarian cancer patient derived TC-210 showed anti-tumor activity. FIG. 9A shows transduction efficiency of T cells derived from ovarian cancer patients.

FIG. 9B shows average tumor volumes measured in MSTO^{MSLN} xenograft model by caliper. Pt, patient; NT, non-transduced T cells.

FIG. 10 depicts experimental data showing patient derived activated T cells (e.g., donor 21, donor 23, and donor 24) transduced with lentivirus encoding TRuCs (TC-210) or CAR (BBζ CAR). The T cells were expanded. The transduction efficiency and surface expression level of the TRuCs and CARs was detected by MSLN-Fc binding using flow cytometry. NT T cells served as negative control.

FIG. 11A depicts experimental data showing mature and immature dendritic cells differentiation. Immature and mature DC were grown 7-8 days post culture. Surface levels expression of activation/co-stimulation marker on mature DC compared to immature DC in Donor 21. Gating on live cells was performed using isotype control for mature DC and immature DCs.

FIG. 11B depicts experimental data showing mature and immature dendritic cells differentiation. Immature and mature DC were grown 7-8 days post culture. Surface levels expression of activation/co-stimulation marker on mature DC compared to immature DC in Donor 23. Gating on live cells was performed using isotype control for mature DC and immature DCs.

FIG. 11C depicts experimental data showing mature and immature dendritic cells differentiation. Immature and mature DC were grown 7-8 days post culture. Surface levels expression of activation/co-stimulation marker on mature DC compared to immature DC in Donor 24. Gating on live cells was performed using isotype control for mature DC and immature DCs.

FIG. 12A depicts experimental data showing TC-210 release less IL-6 than CAR-T cells in the presence of APCs In vitro. IL-6 levels from NT, TC-210 or MH1 41BBζ CAR transduced T cells from representative Donor 21 were co-cultured with MSTO^{MSLN} in the presence or absence of immature or mature DCs. Culture supernatants were harvested at various time points were subjected to cytokine analysis by luminex based immuno assay. Two-way ANOVA statistics used to compare cytokine levels between TRuCs and CARs (*p-value <0.5, **p-value <0.01, ***p-value <0.001).

FIG. 12B depicts experimental data showing TC-210 release less IL-6 than CAR-T cells in the presence of APCs In vitro. IL-6 levels from NT, TC-210 or MH1 41BBζ CAR transduced T cells from representative Donor 23 were co-cultured with MSTO^{MSLN} in the presence or absence of immature or mature DCs. Culture supernatants were harvested at various time points were subjected to cytokine analysis by luminex based immuno assay. Two-way ANOVA statistics used to compare cytokine levels between TRuCs and CARs (*p-value <0.5, **p-value <0.01, ***p-value <0.001).

FIG. 12C depicts experimental data showing TC-210 release less IL-6 than CAR-T cells in the presence of APCs in vitro. IL-6 levels from NT, TC-210 or MH1 41BBζ CAR transduced T cells from representative Donor 24 were co-cultured with MSTO^{MSLN} in the presence or absence of immature or mature DCs. Culture supernatants were harvested at various time points were subjected to cytokine analysis by luminex based immuno assay. Two-way ANOVA statistics used to compare cytokine levels between TRuCs and CARs (*p-value <0.5, **p-value <0.01, ***p-value <0.001).

FIG. 13A depicts experimental data showing TC-210 release less CRS-related cytokines, e.g., IL-6, than CAR-T cells in the presence of APCs in vitro. NT, TC-210 or MH1 41BBζ CAR transduced T cells from representative Donor 24 were co-cultured with MSTO^{MSLN} in the presence or absence of immature or mature DCs. Culture supernatants were harvested at various time points were subjected to cytokine analysis by luminex based immuno assay. Two-way ANOVA statistics used to compare cytokine levels between TRuCs and CARs (*p-value <0.5, **p-value <0.01, ***p-value <0.001).

FIG. 13B depicts experimental data showing TC-210 release less CRS-related cytokines, e.g., IFN-g, than CAR-T cells in the presence of APCs in vitro. NT, TC-210 or MH1 41BBζ CAR transduced T cells from representative Donor 24 were co-cultured with MSTO^{MSLN} in the presence or absence of immature or mature DCs. Culture supernatants were harvested at various time points were subjected to cytokine analysis by luminex based immuno assay. Two-way ANOVA statistics used to compare cytokine levels between TRuCs and CARs (*p-value <0.5, **p-value <0.01, ***p-value <0.001).

FIG. 13C depicts experimental data showing TC-210 release less CRS-related cytokines, e.g., IL-1β, than CAR-T cells in the presence of APCs in vitro. NT, TC-210 or MH1 41BBζ CAR transduced T cells from representative Donor 24 were co-cultured with MSTO^{MSLN} in the presence or absence of immature or mature DCs. Culture supernatants were harvested at various time points were subjected to cytokine analysis by luminex based immuno assay. Two-way ANOVA statistics used to compare cytokine levels between TRuCs and CARs (*p-value <0.5, **p-value <0.01, ***p-value <0.001).

FIG. 13D depicts experimental data showing TC-210 release less CRS-related cytokines, e.g., TNF-α, than CAR-T cells in the presence of APCs in vitro. NT, TC-210 or MH1 41BBζ CAR transduced T cells from representative Donor 24 were co-cultured with MSTO^{MSLN} in the presence or absence of immature or mature DCs. Culture supernatants were harvested at various time points were subjected to cytokine analysis by luminex based immuno assay. Two-way ANOVA statistics used to compare cytokine levels between TRuCs and CARs (*p-value <0.5, **p-value <0.01, ***p-value <0.001).

FIG. 13E depicts experimental data showing TC-210 release less CRS-related cytokines, e.g., IL-2, than CAR-T cells in the presence of APCs in vitro. NT, TC-210 or MH1 41BBζ CAR transduced T cells from representative Donor 24 were co-cultured with MSTO^{MSLN} in the presence or absence of immature or mature DCs. Culture supernatants were harvested at various time points were subjected to cytokine analysis by luminex based immuno assay. Two-way ANOVA statistics used to compare cytokine levels between TRuCs and CARs (*p-value <0.5, **p-value <0.01, ***p-value <0.001).

FIG. 13F depicts experimental data showing TC-210 release less CRS-related cytokines, e.g., GM-CSF, than CAR-T cells in the presence of APCs in vitro. NT, TC-210 or MH1 41BBζ CAR transduced T cells from representative Donor 24 were co-cultured with MSTO^{MSLN} in the presence or absence of immature or mature DCs. Culture supernatants were harvested at various time points were subjected to cytokine analysis by luminex based immuno assay. Two-way ANOVA statistics used to compare cytokine levels between TRuCs and CARs (*p-value <0.5, **p-value <0.01, ***p-value <0.001).

FIG. 14 depicts experimental data showing TC-210 express lower cytokines mRNA levels than BBζ CAR-T cells in vivo. Heatmap representing fold change in selected cytokine gene expression from TC-210 and BBζ CAR-T cells obtained from mouse, seven days post T cell infusion in MSTO^{MSLN} tumor model in vivo. The cytokine genes were normalized to seven day post-infused NT T cells.

FIG. 15 depicts examples of lentivirus constructs of anti-MSLN TFP. The lentivirus constructs were cloned into p526 lentivector via XbaI and EcoRI restriction sides.

FIG. 16 depicts TFP and CAR expression on T cell surface. NT represents negative controls. MH1e represents T cells expressing anti-MSLN CD3ε TFP receptors. MH1-CD28z represents T cells expressing anti-MSLN CD28ζ CAR receptors. MH1-41BBz represents T cells expressing anti-MSLN 41BBζ CAR receptors. The expression of TFP or CAR receptors on the cell surface were tested in three different donors, including donor 14, donor 15, and donor 17.

FIG. 17A depicts experimental data showing activation of anti-MSLN T cells from donor 14 in the presence of plate-bound mesothelin antigen.

FIG. 17B depicts experimental data showing activation of anti-MSLN T cells from donor 15 in the presence of plate-bound mesothelin antigen.

FIG. 17C depicts experimental data showing activation of anti-MSLN T cells from donor 17 in the presence of plate-bound mesothelin antigen.

FIG. 18 depicts log counts of genes expressed before and after activation.

FIG. 19 depicts a schematic of data analysis methodology. TRuC represents T cells expressing TFP receptors. 28z represents T cells expressing CD28ζ CAR receptors. 41BBz represents T cells expressing 41BBζ CAR receptors.

FIG. 20A depicts a heat map showing gene expression profile in untransduced cells (e.g., UTD_D14, UTD_D15, UTD_D17), anti-MSLN TFP transduced cells (e.g., TRuC_D14, TRuC_D15, TRuC_D17), and anti-MSLN CAR transduced cells (e.g., 28z_D14, 28z_D15, 28z_D17, 41BBz_D14, 41BBz_D15, 41BBz_D17).

FIG. 20B depicts a heat map showing gene expression profile in anti-MSLN TFP transduced cells (e.g., TRuC_D14, TRuC_D15, TRuC_D17), and anti-MSLN CAR transduced cells (e.g., 28z_D14, 28z_D15, 28z_D17).

FIG. 20C depicts a heat map showing gene expression profile in anti-MSLN TFP transduced cells (e.g., TRuC_D14, TRuC_D15, TRuC_D17), and anti-MSLN CAR transduced cells (41BBz_D14, 41BBz_D15, 41BBz_D17).

FIG. 21A depicts a heat map showing gene expression profile in untransduced cells (e.g., UTD_D14, UTD_D15, UTD_D17), anti-MSLN TFP transduced cells (e.g., TRuC_D14, TRuC_D15, TRuC_D17), and anti-MSLN CAR transduced cells (e.g., 28z_D14, 28z_D15, 28z_D17, 41BBz_D14, 41BBz_D15, 41BBz_D17). These genes have functions in antigen presentation.

FIG. 21B depicts a heat map showing gene expression profile in untransduced cells (e.g., UTD_D14, UTD_D15, UTD_D17), anti-MSLN TFP transduced cells (e.g., TRuC_D14, TRuC_D15, TRuC_D17), and anti-MSLN CAR transduced cells (e.g., 28z_D14, 28z_D15, 28z_D17, 41BBz_D14, 41BBz_D15, 41BBz_D17). These genes have functions in TCR singling.

FIG. 21C depicts a heat map showing gene expression profile in untransduced cells (e.g., UTD_D14, UTD_D15, UTD_D17), anti-MSLN TFP transduced cells (e.g., TRuC_D14, TRuC_D15, TRuC_D17), and anti-MSLN CAR transduced cells (e.g., 28z_D14, 28z_D15, 28z_D17, 41BBz_D14, 41BBz_D15, 41BBz_D17). These genes have functions in homeostasis or metabolism.

FIG. 21D depicts a heat map showing gene expression profile in untransduced cells (e.g., UTD_D14, UTD_D15, UTD_D17), anti-MSLN TFP transduced cells (e.g., TRuC_D14, TRuC_D15, TRuC_D17), and anti-MSLN CAR transduced cells (e.g., 28z_D14, 28z_D15, 28z_D17, 41BBz_D14, 41BBz_D15, 41BBz_D17). These genes have functions in MMP and adhesion, toll-like receptor (TLR) associated pathways, or tumor necrosis factor receptor (TNFR) associated pathways.

FIG. 21E depicts a heat map showing gene expression profile in untransduced cells (e.g., UTD_D14, UTD_D15, UTD_D17), anti-MSLN TFP transduced cells (e.g., TRuC_D14, TRuC_D15, TRuC_D17), and anti-MSLN CAR transduced cells (e.g., 28z_D14, 28z_D15, 28z_D17, 41BBz_D14, 41BBz_D15, 41BBz_D17). These genes have functions in chemokines.

FIG. 21F depicts a heat map showing gene expression profile in untransduced cells (e.g., UTD_D14, UTD_D15, UTD_D17), anti-MSLN TFP transduced cells (e.g., TRuC_D14, TRuC_D15, TRuC_D17), and anti-MSLN CAR transduced cells (e.g., 28z_D14, 28z_D15, 28z_D17, 41BBz_D14, 41BBz_D15, 41BBz_D17). These genes have functions in cytokine signaling.

FIG. 22 depicts a heat map showing gene expression profile in anti-MSLN TFP transduced cells (e.g., TRuC_D14, TRuC_D15, TRuC_D17), and anti-MSLN CAR transduced cells (e.g., 28z_D14, 28z_D15, 28z_D17). The genes are grouped based on their functionality.

FIG. 23A depicts a heat map showing gene expression profile in anti-MSLN TFP transduced cells (e.g., TRuC_D14, TRuC_D15, TRuC_D17), and anti-MSLN CAR transduced cells (e.g., 41BBz_D14, 41BBz_D15, 41BBz_D17). The genes have functions in TCR signaling.

FIG. 23B depicts a heat map showing gene expression profile in anti-MSLN TFP transduced cells (e.g., TRuC_D14, TRuC_D15, TRuC_D17), and anti-MSLN CAR transduced cells (e.g., 41BBz_D14, 41BBz_D15, 41BBz_D17). The genes have functions in chemokine signaling.

FIG. 23C depicts a heat map showing gene expression profile in anti-MSLN TFP transduced cells (e.g., TRuC_D14, TRuC_D15, TRuC_D17), and anti-MSLN CAR transduced cells (e.g., 41BBz_D14, 41BBz_D15, 41BBz_D17). These genes have functions in cytokine signaling.

FIG. 23D depicts a heat map showing gene expression profile in anti-MSLN TFP transduced cells (e.g., TRuC_D14, TRuC_D15, TRuC_D17), and anti-MSLN CAR transduced cells (e.g., 41BBz_D14, 41BBz_D15, 41BBz_D17). The genes have functions in cell signaling.

FIG. 23E depicts a heat map showing gene expression profile in anti-MSLN TFP transduced cells (e.g., TRuC_D14, TRuC_D15, TRuC_D17), and anti-MSLN CAR transduced cells (e.g., 41BBz_D14, 41BBz_D15, 41BBz_D17). The gene have functions in antigen presentation, hemostasis, metabolism, MMP and ECM interactions, or are associated with TLR or TNFR.

FIG. 24 depicts examples of lentivirus constructs of CD19-specific TFP. The lentivirus constructs were cloned into p526 lentivector via XbaI and EcoRI restriction sites.

FIG. 25 depicts a schematic of experimental outline. T cells were co-cultured with Raji cells at 1:1 E:T ratio for 4 hours and sorted using anti-CD19 beads to isolate T cells.

FIG. 26A depicts CD19-specific TFP receptor surface expression on T cells from donor 12 before and after co-culture with Raji cells.

FIG. 26B depicts CD19-specific TFP receptor surface expression on T cells from donor 14 before and after co-culture with Raji cells.

FIG. 26C depicts CD19-specific TFP receptor surface expression on T cells from donor 15 before and after co-culture with Raji cells.

FIG. 27 depicts experimental data showing the presence of CD19+ Raji cells post-T cell enrichment.

FIG. 28A depicts T cell activation post tumor cell co-culture. 30-40% CD 69 expression showed T cell activation post tumor cell co-culture for 4 hours. CD69 and GFP expression on T cells before co-culture, cells in sorting column, and eluted T cells post co-culture for donor 12 are represented.

FIG. 28B depicts T cell activation post tumor cell co-culture. 30-40% CD 69 expression showed T cell activation post tumor cell co-culture for 4 hours. CD69 and GFP expression on T cells before co-culture, cells in sorting column, and eluted T cells post co-culture for donor 14 are represented.

FIG. 28C depicts T cell activation post tumor cell co-culture. 30-40% CD 69 expression showed T cell activation post tumor cell co-culture for 4 hours. CD69 and GFP expression on T cells before co-culture, cells in sorting column, and eluted T cells post co-culture for donor 15 are represented.

FIG. 29A depicts log counts of genes expressed before activation in negative control T cells, T cells expressing CD19-specific 41BBζ CAR (41BBz), T cells expressing CD19-specific CD3ε TFP (CD3e), T cells expressing CD28ζ CAR (CD28z).

FIG. 29B depicts log counts of genes expressed after activation in negative control T cells, T cells expressing CD19-specific 41BBζ CAR (41BBz), T cells expressing CD19-specific CD3ε TFP (CD3e), T cells expressing CD28ζ CAR (CD28z).

FIG. 30 depicts a schematic of data analysis methodology.

FIG. 31A depicts normalization of gene count based on endogenous control and p-value. The heat map represents genes that are significantly upregulated or down regulated post tumor cell-mediated activation. The figure depicts a heat map showing gene expression profile in untransduced cells (e.g., UTD_D14, UTD_D15, UTD_D17), CD19-specific TFP transduced cells (e.g., TRuC_D14, TRuC_D15, TRuC_D17), and CD19-specific CAR transduced cells (e.g., 28z_D14, 28z_D15, 28z_D17, 41BBz_D14, 41BBz_D15, 41BBz_D17).

FIG. 31B depicts normalization of gene count based on endogenous control and p-value. The heat map represents genes that are significantly upregulated or down regulated post tumor cell-mediated activation. The figure depicts a heat map showing gene expression profile in CD19-specific TFP transduced cells (e.g., TRuC_D14, TRuC_D15, TRuC_D17), and CD19-specific CAR transduced cells (e.g., 28z_D14, 28z_D15, 28z_D17).

FIG. 31C depicts normalization of gene count based on endogenous control and p-value. The heat map represents genes that are significantly upregulated or down regulated post tumor cell-mediated activation. The figure depicts a heat map showing gene expression profile in CD19-specific TFP transduced cells (e.g., TRuC_D14, TRuC_D15, TRuC_D17), and CD19-specific CAR transduced cells (41BBz_D14, 41BBz_D15, 41BBz_D17).

FIG. 32 depicts activated CD19-specific TFP expressing T cells show different gene expression patterns than CD19-specific CAR-T Cells after 4-hour stimulation.

FIG. 33A depicts a heat map showing gene expression profile in CD19-specific TFP transduced cells, and CD19-specific CD28ζ CAR transduced cells. The genes have functions in cell signaling.

FIG. 33B depicts a heat map showing gene expression profile in CD19-specific TFP transduced cells, and CD19-specific CD28ζ CAR transduced cells. The genes have functions in chemokine signaling.

FIG. 33C depicts a heat map showing gene expression profile in CD19-specific TFP transduced cells, and CD19-specific CD28ζ CAR transduced cells. The genes have functions in cytokine signaling.

FIG. 33D depicts a heat map showing gene expression profile in CD19-specific CD3ε TFP transduced cells (e.g., CD3e_12, CD3e_14, CD3e_15), and CD19-specific 41BBζ CAR transduced cells (e.g., 41BBz_12, 41BBz_14, 41BBz_15). The genes have functions in TCR signaling.

FIG. 33E depicts a heat map showing gene expression profile in CD19-specific CD3ε TFP transduced cells (e.g., CD3e_12, CD3e_14, CD3e_15), and CD19-specific 41BBζ CAR transduced cells (e.g., 41BBz_12, 41BBz_14, 41BBz_15). The genes have functions in chemokine signaling.

FIG. 33F depicts a heat map showing gene expression profile in CD19-specific CD3ε TFP transduced cells (e.g., CD3e_12, CD3e_14, CD3e_15), and CD19-specific 41BBζ CAR transduced cells (e.g., 41BBz_12, 41BBz_14, 41BBz_15). The genes have functions in cytokine signaling.

FIG. 34A depicts data showing pathway involvement of upregulated genes in CD19-specific CD3ε TFP transduced cells, CD19-specific CD28ζ CAR transduced cells, and CD19-specific 41BBζ CAR transduced cells.

FIG. 34B depicts data showing pathway involvement of downregulated genes in CD19-specific CD3ε TFP transduced cells, CD19-specific CD28ζ CAR transduced cells, and CD19-specific 41BBζ CAR transduced cells.

FIG. 35A depicts experimental data showing cell surface expression of TFP receptors in cells transduced with different variants of CD19-specific TFP constructs. α, β, δ, γ, and ε represent different chains of TCR with which anti-CD19 binding domain is fused with.

FIG. 35B depicts experimental data showing cell surface expression of CAR receptors in cells transduced with different variants of CD19-specific CAR constructs.

FIG. 36 depicts experimental data showing co-immunoprecipitation of different TFP T variants with CD3ε chain. The Western Blots were blotted with either Anti-TCRα, Anti-TCRβ, anti-CD3ε, Anti-CD3γ, or Anti-CD3ζ.

FIG. 37A depicts phosphoprotein analysis with five T cell donors showing activated TFP T cells signal differently than activated CAR-T cells. The results show at least a 3.2 fold increase in phosphorylation of CD3ε. As used herein, “TRuC” represents “TFP”.

FIG. 37B depicts phosphoprotein analysis with five T cell donors showing activated TFP T cells signal differently than activated CAR-T cells. The results show at least a 1.5 fold increase in phosphorylation of LAT. As used herein, “TRuC” represents “TFP”.

FIG. 37C depicts phosphoprotein analysis with five T cell donors showing activated TFP T cells signal differently than activated CAR-T cells. The results show at least a 1.3 fold increase in phosphorylation of CREB. As used herein, “TRuC” represents “TFP”.

FIG. 38A depicts experimental data showing impedance-based kinetic cell lysis assay. The TFP T cells show potent target cell killing activity. As used herein, “TRuC” represents “TFP”.

FIG. 38B depicts experimental data showing Raji-Luc lysis assay. The TFP T cells show potent target cell killing activity. As used herein, “TRuC™” represents “TFP”.

FIG. 39 depicts experimental data showing TFP T cells secrete less cytokines than CAR-T cells. As used herein, “TRuC” represents “TFP”.

FIG. 40A depicts an example experimental design for tumor volume measurement.

FIG. 40B depicts experimental data of the experiment in FIG. 40A showing TFP T cells demonstrate higher anti-tumor activity in Raji subcutaneous model compared to CAR-T cells.

FIG. 41 depicts experimental data showing survival percentage over time in Raji xenograft mouse model.

FIG. 42 depicts experimental data showing impedance-based kinetic cell lysis assay. The data show that MSLN-specific TFP T and CAR-T cells appear equally potent in this assay.

FIG. 43A depicts experimental data showing co-immunoprecipitation of different TFP T variants with anti-scFv antibody. As used herein, “TRuC” represents “TFP”. The Western Blots were blotted with either Anti-TCRα, Anti-TCRβ, anti-CD3ε, Anti-CD3γ, Anti-CD3δ, or Anti-CD3ζ

FIG. 43B depicts experimental data showing TFP variants are integrated into the endogenous TCR complex. As used herein, “TRuC” represents “TFP”.

### DETAILED DESCRIPTION

In one aspect, described herein are isolated nucleic acid molecules encoding a T cell Receptor (TCR) fusion protein (TFP) that comprise a TCR subunit and a human or humanized antibody domain comprising an anti-mesothelin binding domain. In some embodiments, the TCR subunit comprises a TCR extracellular domain. In other embodiments, the TCR subunit comprises a TCR transmembrane domain. In yet other embodiments, the TCR subunit comprises a TCR intracellular domain. In further embodiments, the TCR subunit comprises (i) a TCR extracellular domain, (ii) a TCR transmembrane domain, and (iii) a TCR intracellular domain, wherein at least two of (i), (ii), and (iii) are from the same TCR subunit. In yet further embodiments, the TCR subunit comprises a TCR intracellular domain comprising a stimulatory domain selected from an intracellular signaling domain of CD3 epsilon, CD3 gamma or CD3 delta, or an amino acid sequence having at least one, two or three modifications thereto. In yet further embodiments, the TCR subunit comprises an intracellular domain comprising a stimulatory domain selected from a functional signaling domain of 4-1BB and/or a functional signaling domain of CD3 zeta, or an amino acid sequence having at least one, two or three modifications thereto.

In some embodiments, the isolated nucleic acid molecules comprise (i) a light chain (LC) CDR1, LC CDR2 and LC CDR3 of any anti-mesothelin light chain binding domain amino acid sequence provided herein, and/or (ii) a heavy chain (HC) CDR1, HC CDR2 and HC CDR3 of any anti-mesothelin heavy chain binding domain amino acid sequence provided herein.

In some embodiments, the light chain variable region comprises an amino acid sequence having at least one, two or three modifications but not more than 30, 20 or 10 modifications of an amino acid sequence of a light chain variable region provided herein, or a sequence with 95-99% identity to an amino acid sequence provided herein. In other embodiments, the heavy chain variable region comprises an amino acid sequence having at least one, two or three modifications but not more than 30, 20 or 10 modifications of an amino acid sequence of a heavy chain variable region provided herein, or a sequence with 95-99% identity to an amino acid sequence provided herein.

In some embodiments, the TFP includes an extracellular domain of a TCR subunit that comprises an extracellular domain or portion thereof of a protein selected from the group consisting of the alpha or beta chain of the T cell receptor, CD3 delta, CD3 epsilon, or CD3 gamma, or a functional fragment thereof, or an amino acid sequence having at least one, two or three modifications but not more than 20, 10 or 5 modifications thereto. In other embodiments, the encoded TFP includes a transmembrane domain that comprises a transmembrane domain of a protein selected from the group consisting of the alpha, beta chain of the TCR or TCR subunits CD3 epsilon, CD3 gamma and CD3 delta, or a functional fragment thereof, or an amino acid sequence having at least one, two or three modifications but not more than 20, 10 or 5 modifications thereto.

In some embodiments, the encoded TFP includes a transmembrane domain that comprises a transmembrane domain of a protein selected from the group consisting of the alpha, beta or zeta chain of the TCR or CD3 epsilon, CD3 gamma and CD3 delta CD45, CD2, CD4, CD5, CD8, CD9, CD16, CD22, CD33, CD28, CD37, CD64, CD80, CD86, CD134, CD137 and CD154, or a functional fragment thereof, or an amino acid sequence having at least one, two or three modifications but not more than 20, 10 or 5 modifications thereto.

In some embodiments, the encoded anti-mesothelin binding domain is connected to the TCR extracellular domain by a linker sequence. In some instances, the encoded linker sequence comprises (G₄S)ₙ, wherein n=1 to 4. In some instances, the encoded linker sequence comprises a long linker (LL) sequence. In some instances, the encoded long linker sequence comprises (G₄S)ₙ, wherein n=2 to 4. In some instances, the encoded linker sequence comprises a short linker (SL) sequence. In some instances, the encoded short linker sequence comprises (G₄S)ₙ, wherein n=1 to 3.

In some embodiments, the isolated nucleic acid molecules further comprise a sequence encoding a costimulatory domain. In some instances, the costimulatory domain is a functional signaling domain obtained from a protein selected from the group consisting of DAP10, DAP12, CD30, LIGHT, OX40, CD2, CD27, CD28, CDS, ICAM-1, LFA-1 (CD11a/CD18), ICOS (CD278), and 4-1BB (CD137), or an amino acid sequence having at least one, two or three modifications but not more than 20, 10 or 5 modifications thereto.

In some embodiments, the isolated nucleic acid molecules further comprise a leader sequence.

Also provided herein are isolated polypeptide molecules encoded by any of the previously described nucleic acid molecules.

Also provided herein in another aspect, are isolated T cell receptor fusion protein (TFP) molecules that comprise a human or humanized anti-mesothelin binding domain, a TCR extracellular domain, a transmembrane domain, and an intracellular domain. In some embodiments, the isolated TFP molecules comprises an antibody or antibody fragment comprising a human or humanized anti-mesothelin binding domain, a TCR extracellular domain, a transmembrane domain, and an intracellular domain.

In some embodiments, the human or humanized antibody domain comprises an antibody fragment. In some embodiments, the human or humanized antibody domain comprises a scFv or a V_{H} domain.

In some embodiments, the anti-mesothelin binding domain is a scFv or a V_{H} domain. In other embodiments, the anti-mesothelin binding domain comprises a light chain and a heavy chain of an amino acid sequence provided herein, or a functional fragment thereof, or an amino acid sequence having at least one, two or three modifications but not more than 30, 20 or 10 modifications of an amino acid sequence of a light chain variable region provided herein, or a sequence with 95-99% identity with an amino acid sequence provided herein.

In some embodiments, the isolated TFP molecules comprise a TCR extracellular domain that comprises an extracellular domain or portion thereof of a protein selected from the group consisting of the alpha or beta chain of the T cell receptor, CD3 delta, CD3 epsilon, or CD3 gamma, or an amino acid sequence having at least one, two or three modifications but not more than 20, 10 or 5 modifications thereto.

In some embodiments, the anti-mesothelin binding domain is connected to the TCR extracellular domain by a linker sequence. In some instances, the linker region comprises (G₄S)ₙ, wherein n=1 to 4. In some instances, the linker sequence comprises a long linker (LL) sequence. In some instances, the long linker sequence comprises (G₄S)ₙ, wherein n=2 to 4. In some instances, the linker sequence comprises a short linker (SL) sequence. In some instances, the short linker sequence comprises (G₄S)ₙ, wherein n=1 to 3.

In some embodiments, the isolated TFP molecules further comprise a sequence encoding a costimulatory domain. In other embodiments, the isolated TFP molecules further comprise a sequence encoding an intracellular signaling domain. In yet other embodiments, the isolated TFP molecules further comprise a leader sequence.

Also provided herein are vectors that comprise a nucleic acid molecule encoding any of the previously described TFP molecules. In some embodiments, the vector is selected from the group consisting of a DNA, a RNA, a plasmid, a lentivirus vector, adenoviral vector, or a retrovirus vector. In some embodiments, the vector further comprises a promoter. In some embodiments, the vector is an in vitro transcribed vector. In some embodiments, a nucleic acid sequence in the vector further comprises a poly(A) tail. In some embodiments, a nucleic acid sequence in the vector further comprises a 3′UTR.

Also provided herein are cells that comprise any of the described vectors. In some embodiments, the cell is a human T cell. In some embodiments, the cell is a CD8+ or CD4+ T cell. In other embodiments, the cells further comprise a nucleic acid encoding an inhibitory molecule that comprises a first polypeptide that comprises at least a portion of an inhibitory molecule, associated with a second polypeptide that comprises a positive signal from an intracellular signaling domain. In some instances, the inhibitory molecule comprise first polypeptide that comprises at least a portion of PD1 and a second polypeptide comprising a costimulatory domain and primary signaling domain.

In another aspect, provided herein are isolated TFP molecules that comprise a human or humanized anti-mesothelin binding domain, a TCR extracellular domain, a transmembrane domain, and an intracellular signaling domain, wherein the TFP molecule is capable of functionally interacting with an endogenous TCR complex and/or at least one endogenous TCR polypeptide.

In another aspect, provided herein are isolated TFP molecules that comprise a human or humanized anti-mesothelin binding domain, a TCR extracellular domain, a transmembrane domain, and an intracellular signaling domain, wherein the TFP molecule is capable of functionally integrating into an endogenous TCR complex.

In another aspect, provided herein are human CD8+ or CD4+ T cells that comprise at least two TFP molecules, the TFP molecules comprising a human or humanized anti-mesothelin binding domain, a TCR extracellular domain, a transmembrane domain, and an intracellular domain, wherein the TFP molecule is capable of functionally interacting with an endogenous TCR complex and/or at least one endogenous TCR polypeptide in, at and/or on the surface of the human CD8+ or CD4+ T cell.

In another aspect, provided herein are protein complexes that comprise i) a TFP molecule comprising a human or humanized anti-mesothelin binding domain, a TCR extracellular domain, a transmembrane domain, and an intracellular domain; and ii) at least one endogenous TCR complex.

In some embodiments, the TCR comprises an extracellular domain or portion thereof of a protein selected from the group consisting of the alpha or beta chain of the T cell receptor, CD3 delta, CD3 epsilon, or CD3 gamma. In some embodiments, the anti-mesothelin binding domain is connected to the TCR extracellular domain by a linker sequence. In some instances, the linker region comprises (G₄S)ₙ, wherein n=1 to 4. In some instances, the linker sequence comprises a long linker (LL) sequence. In some instances, the long linker sequence comprises (G₄S)ₙ, wherein n=2 to 4. In some instances, the linker sequence comprises a short linker (SL) sequence. In some instances, the short linker sequence comprises (G₄S)ₙ, wherein n=1 to 3.

Also provided herein are human CD8+ or CD4+ T cells that comprise at least two different TFP proteins per any of the described protein complexes.

In another aspect, provided herein is a population of human CD8+ or CD4+ T cells, wherein the T cells of the population individually or collectively comprise at least two TFP molecules, the TFP molecules comprising a human or humanized anti-mesothelin binding domain, a TCR extracellular domain, a transmembrane domain, and an intracellular domain, wherein the TFP molecule is capable of functionally interacting with an endogenous TCR complex and/or at least one endogenous TCR polypeptide in, at and/or on the surface of the human CD8+ or CD4+ T cell.

In another aspect, provided herein is a population of human CD8+ or CD4+ T cells, wherein the T cells of the population individually or collectively comprise at least two TFP molecules encoded by an isolated nucleic acid molecule provided herein.

In another aspect, provided herein are methods of making a cell comprising transducing a T cell with any of the described vectors.

In another aspect, provided herein are methods of generating a population of RNA-engineered cells that comprise introducing an in vitro transcribed RNA or synthetic RNA into a cell, where the RNA comprises a nucleic acid encoding any of the described TFP molecules.

In another aspect, provided herein are methods of providing an anti-tumor immunity in a mammal that comprise administering to the mammal an effective amount of a cell expressing any of the described TFP molecules. In some embodiments, the cell is an autologous T cell. In some embodiments, the cell is an allogeneic T cell. In some embodiments, the mammal is a human.

In another aspect, provided herein are methods of treating a mammal having a disease associated with expression of mesothelin that comprise administering to the mammal an effective amount of the cell of comprising any of the described TFP molecules. In some embodiments, the disease associated with mesothelin expression is selected from a proliferative disease such as a cancer or malignancy or a precancerous condition such as a pancreatic cancer, an ovarian cancer, a stomach cancer, a lung cancer, or an endometrial cancer, or is a non-cancer related indication associated with expression of mesothelin.

In some embodiments, the cells expressing any of the described TFP molecules are administered in combination with an agent that ameliorates one or more side effects associated with administration of a cell expressing a TFP molecule. In some embodiments, the cells expressing any of the described TFP molecules are administered in combination with an agent that treats the disease associated with mesothelin.

Also provided herein are any of the described isolated nucleic acid molecules, any of the described isolated polypeptide molecules, any of the described isolated TFPs, any of the described protein complexes, any of the described vectors or any of the described cells for use as a medicament

### Definitions

Unless defined otherwise, all technical and scientific terms used herein have the same meaning as commonly understood by one of ordinary skill in the art to which the invention pertains.

The term “a” and “an” refers to one or to more than one (i.e., to at least one) of the grammatical object of the article. By way of example, “an element” means one element or more than one element.

As used herein, “about” can mean plus or minus less than 1 or 1, 2, 3, 4, 5, 6, 7, 8, 9, 10, 11, 12, 13, 14, 15, 16, 17, 18, 19, 20, 25, 30, or greater than 30 percent, depending upon the situation and known or knowable by one skilled in the art.

As used herein the specification, “subject” or “subjects” or “individuals” may include, but are not limited to, mammals such as humans or non-human mammals, e.g., domesticated, agricultural or wild, animals, as well as birds, and aquatic animals. “Patients” are subjects suffering from or at risk of developing a disease, disorder or condition or otherwise in need of the compositions and methods provided herein.

As used herein, “treating” or “treatment” refers to any indicia of success in the treatment or amelioration of the disease or condition. Treating can include, for example, reducing, delaying or alleviating the severity of one or more symptoms of the disease or condition, or it can include reducing the frequency with which symptoms of a disease, defect, disorder, or adverse condition, and the like, are experienced by a patient. As used herein, “treat or prevent” is sometimes used herein to refer to a method that results in some level of treatment or amelioration of the disease or condition, and contemplates a range of results directed to that end, including but not restricted to prevention of the condition entirely.

As used herein, “preventing” refers to the prevention of the disease or condition, e.g., tumor formation, in the patient. For example, if an individual at risk of developing a tumor or other form of cancer is treated with the methods of the present invention and does not later develop the tumor or other form of cancer, then the disease has been prevented, at least over a period of time, in that individual.

As used herein, a “therapeutically effective amount” is the amount of a composition or an active component thereof sufficient to provide a beneficial effect or to otherwise reduce a detrimental non-beneficial event to the individual to whom the composition is administered. By “therapeutically effective dose” herein is meant a dose that produces one or more desired or desirable (e.g., beneficial) effects for which it is administered, such administration occurring one or more times over a given period of time. The exact dose will depend on the purpose of the treatment, and will be ascertainable by one skilled in the art using known techniques (see, e.g. Lieberman, Pharmaceutical Dosage Forms (vols. 1-3, 1992); Lloyd, The Art, Science and Technology of Pharmaceutical Compounding (1999); and Pickar, Dosage Calculations (1999))

As used herein, a “T cell receptor (TCR) fusion protein” or “TFP” includes a recombinant polypeptide derived from the various polypeptides comprising the TCR that is generally capable of i) binding to a surface antigen on target cells and ii) interacting with other polypeptide components of the intact TCR complex, typically when co-located in or on the surface of a T cell. A “TFP T cell” is a T cell that has been transduced according to the methods disclosed herein and that expresses a TFP, e.g., incorporated into the natural TCR. In some embodiments, the T cell is a CD4+ T cell, a CD8+ T cell, or a CD4+/CD8+ T cell. In some embodiments, the TFP T cell is an NK cell. In some embodiments,

As used herein, the term “mesothelin” also known as MSLN or CAK1 antigen or Pre-pro-megakaryocyte-potentiating factor, refers to the protein that in humans is encoded by the MSLN (or Megakaryocyte-potentiating factor (MPF)) gene. Mesothelin is a 40 kDa protein present on normal mesothelial cells and overexpressed in several human tumors, including mesothelioma and ovarian and pancreatic adenocarcinoma. The mesothelin gene encodes a precursor protein that is processed to yield mesothelin which is attached to the cell membrane by a glycophosphatidylinositol linkage and a 31-kDa shed fragment named megakaryocyte-potentiating factor (MPF). Mesothelin may be involved in cell adhesion, but its biological function is not known. Mesothelin is a tumour differentiation antigen that is normally present on the mesothelial cells lining the pleura, peritoneum and pericardium. Mesothelin is an antigenic determinant detectable on mesothelioma cells, ovarian cancer cells, pancreatic adenocarcinoma cell and some squamous cell carcinomas (see, e.g., Kojima et al., J. Biol. Chem. 270:21984-21990 (1995) and Onda et al., Clin. Cancer Res. 12:4225-4231 (2006)). Mesothelin interacts with CA125/MUC16 (see, e.g., Rump et al., J. Biol. Chem. 279:9190-9198 (2004) and Ma et al., J. Biol. Chem. 287:33123-33131 (2012)).

The human and murine amino acid and nucleic acid sequences can be found in a public database, such as GenBank, UniProt and Swiss-Prot. For example, the amino acid sequence of human mesothelin can be found as UniProt/Swiss-Prot Accession No. Q13421. The human mesothelin polypeptide canonical sequence is UniProt Accession No. Q13421 (or Q13421-1):

(SEQ ID NO: 15)
MALPTARPLLGSCGTPALGSLLFLLFSLGWVQPSRTLAGETGQEAAPLDG
VLANPPNISSLSPRQLLGFPCAEVSGLSTERVRELAVALAQKNVKLSTEQ
LRCLAHRLSEPPEDLDALPLDLLLFLNPDAFSGPQACTRFFSRITKANVD
LLPRGAPERQRLLPAALACWGVRGSLLSEADVRALGGLACDLPGRFVAES
AEVLLPRLVSCPGPLDQDQQEAARAALQGGGPPYGPPSTWSVSTMDALRG
LLPVLGQPIIRSIPQGIVAAWRQRSSRDPSWRQPERTILRPRFRREVEKT
ACPSGKKAREIDESLIFYKKWELEACVDAALLATQMDRVNAIPFTYEQLD
VLKHKLDELYPQGYPESVIQHLGYLFLKMSPEDIRKWNVTSLETLKALLE
VNKGHEMSPQAPRRPLPQVATLIDRFVKGRGQLDKDTLDTLTAFYPGYLC
SLSPEELSSVPPSSIWAVRPQDLDTCDPRQLDVLYPKARLAFQNMNGSEY
FVKIQSFLGGAPTEDLKALSQQNVSMDLATFMKLRTDAVLPLTVAEVQKL
LGPHVEGLKAEERHRPVRDWILRQRQDDLDTLGLGLQGGIPNGYLVLDLS
MQEALSGTPCLLGPGPVLTVLALLLASTLA.

The nucleotide sequence encoding human mesothelin transcript variant 1 can be found at Accession No. NM005823. The nucleotide sequence encoding human mesothelin transcript variant 2 can be found at Accession No. NM013404. The nucleotide sequence encoding human mesothelin transcript variant 3 can be found at Accession No. NM001177355. Mesothelin is expressed on mesothelioma cells, ovarian cancer cells, pancreatic adenocarcinoma cell and squamous cell carcinomas (see, e.g., Kojima et al., J. Biol. Chem. 270:21984-21990 (1995) and Onda et al., Clin. Cancer Res. 12:4225-4231 (2006)). Other cells that express mesothelin are provided below in the definition of “disease associated with expression of mesothelin.” Mesothelin also interacts with CA125/MUC16 (see, e.g., Rump et al., J. Biol. Chem. 279:9190-9198 (2004) and Ma et al., J. Biol. Chem. 287:33123-33131 (2012)). In one example, the antigen-binding portion of TFPs recognizes and binds an epitope within the extracellular domain of the mesothelin protein as expressed on a normal or malignant mesothelioma cell, ovarian cancer cell, pancreatic adenocarcinoma cell, or squamous cell carcinoma cell.

The term “antibody,” as used herein, refers to a protein, or polypeptide sequences derived from an immunoglobulin molecule, which specifically binds to an antigen. Antibodies can be intact immunoglobulins of polyclonal or monoclonal origin, or fragments thereof and can be derived from natural or from recombinant sources.

The terms “antibody fragment” or “antibody binding domain” refer to at least one portion of an antibody, or recombinant variants thereof, that contains the antigen binding domain, i.e., an antigenic determining variable region of an intact antibody, that is sufficient to confer recognition and specific binding of the antibody fragment to a target, such as an antigen and its defined epitope. Examples of antibody fragments include, but are not limited to, Fab, Fab′, F(ab′)₂, and Fv fragments, single-chain (sc)Fv (“scFv”) antibody fragments, linear antibodies, single domain antibodies (abbreviated “sdAb”) (either V_{L} or V_{H}), camelid V_{HH} domains, and multi-specific antibodies formed from antibody fragments.

The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single polypeptide chain, and wherein the scFv retains the specificity of the intact antibody from which it is derived.

“Heavy chain variable region” or “V_{H}” (or, in the case of single domain antibodies, e.g., nanobodies, “V_{HH}”) with regard to an antibody refers to the fragment of the heavy chain that contains three CDRs interposed between flanking stretches known as framework regions, these framework regions are generally more highly conserved than the CDRs and form a scaffold to support the CDRs.

Unless specified, as used herein a scFv may have the V_{L} and V_{H} variable regions in either order, e.g., with respect to the N-terminal and C-terminal ends of the polypeptide, the scFv may comprise V_{L}-linker-V_{H} or may comprise V_{H}-linker-V_{L}.

The portion of the TFP composition of the invention comprising an antibody or antibody fragment thereof may exist in a variety of forms where the antigen binding domain is expressed as part of a contiguous polypeptide chain including, for example, a single domain antibody fragment (sdAb) or heavy chain antibodies HCAb, a single chain antibody (scFv) derived from a murine, humanized or human antibody (Harlow et al., 1999, In: Using Antibodies: A Laboratory Manual, Cold Spring Harbor Laboratory Press, N.Y.; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). In one aspect, the antigen binding domain of a TFP composition of the invention comprises an antibody fragment. In a further aspect, the TFP comprises an antibody fragment that comprises a scFv or a sdAb.

The term “antibody heavy chain,” refers to the larger of the two types of polypeptide chains present in antibody molecules in their naturally occurring conformations, and which normally determines the class to which the antibody belongs.

The term “antibody light chain,” refers to the smaller of the two types of polypeptide chains present in antibody molecules in their naturally occurring conformations. Kappa (“κ”) and lambda (“λ”) light chains refer to the two major antibody light chain isotypes.

The term “recombinant antibody” refers to an antibody that is generated using recombinant DNA technology, such as, for example, an antibody expressed by a bacteriophage or yeast expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

The term “antigen” or “Ag” refers to a molecule that is capable of being bound specifically by an antibody, or otherwise provokes an immune response. This immune response may involve either antibody production, or the activation of specific immunologically-competent cells, or both.

The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be generated synthesized or can be derived from a biological sample, or might be macromolecule besides a polypeptide. Such a biological sample can include, but is not limited to a tissue sample, a tumor sample, a cell or a fluid with other biological components.

The term “anti-tumor effect” refers to a biological effect which can be manifested by various means, including but not limited to, e.g., a decrease in tumor volume, a decrease in the number of tumor cells, a decrease in the number of metastases, an increase in life expectancy, decrease in tumor cell proliferation, decrease in tumor cell survival, or amelioration of various physiological symptoms associated with the cancerous condition. An “anti-tumor effect” can also be manifested by the ability of the peptides, polynucleotides, cells and antibodies of the invention in prevention of the occurrence of tumor in the first place.

The term “autologous” refers to any material derived from the same individual to whom it is later to be re-introduced into the individual.

The term “allogeneic” refers to any material derived from a different animal of the same species or different patient as the individual to whom the material is introduced. Two or more individuals are said to be allogeneic to one another when the genes at one or more loci are not identical. In some aspects, allogeneic material from individuals of the same species may be sufficiently unlike genetically to interact antigenically.

The term “xenogeneic” refers to a graft derived from an animal of a different species.

The term “cancer” refers to a disease characterized by the rapid and uncontrolled growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, lung cancer, and the like.

The phrase “disease associated with expression of mesothelin” includes, but is not limited to, a disease associated with expression of mesothelin or condition associated with cells which express mesothelin including, e.g., proliferative diseases such as a cancer or malignancy or a precancerous condition In one aspect, the cancer is a mesothelioma. In one aspect, the cancer is a pancreatic cancer. In one aspect, the cancer is an ovarian cancer. In one aspect, the cancer is a stomach cancer. In one aspect, the cancer is a lung cancer. In one aspect, the cancer is an endometrial cancer. Non-cancer related indications associated with expression of mesothelin include, but are not limited to, e.g., autoimmune disease, (e.g., lupus, rheumatoid arthritis, colitis), inflammatory disorders (allergy and asthma), and transplantation.

The term “conservative sequence modifications” refers to amino acid modifications that do not significantly affect or alter the binding characteristics of the antibody or antibody fragment containing the amino acid sequence. Such conservative modifications include amino acid substitutions, additions and deletions. Modifications can be introduced into an antibody or antibody fragment of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within a TFP of the invention can be replaced with other amino acid residues from the same side chain family and the altered TFP can be tested using the functional assays described herein.

The term “stimulation” refers to a primary response induced by binding of a stimulatory domain or stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, and/or reorganization of cytoskeletal structures, and the like.

The term “stimulatory molecule” or “stimulatory domain” refers to a molecule or portion thereof expressed by a T cell that provides the primary cytoplasmic signaling sequence(s) that regulate primary activation of the TCR complex in a stimulatory way for at least some aspect of the T cell signaling pathway. In one aspect, the primary signal is initiated by, for instance, binding of a TCR/CD3 complex with an MHC molecule loaded with peptide, and which leads to mediation of a T cell response, including, but not limited to, proliferation, activation, differentiation, and the like. A primary cytoplasmic signaling sequence (also referred to as a “primary signaling domain”) that acts in a stimulatory manner may contain a signaling motif which is known as immunoreceptor tyrosine-based activation motif or “ITAM”. Examples of an ITAM containing primary cytoplasmic signaling sequence that is of particular use in the invention includes, but is not limited to, those derived from TCR zeta, FcR gamma, FcR beta, CD3 gamma, CD3 delta, CD3 epsilon, CD5, CD22, CD79a, CD79b, CD278 (also known as “ICOS”) and CD66d.

The term “antigen presenting cell” or “APC” refers to an immune system cell such as an accessory cell (e.g., a B-cell, a dendritic cell, and the like) that displays a foreign antigen complexed with major histocompatibility complexes (MHC's) on its surface. T cells may recognize these complexes using their T cell receptors (TCRs). APCs process antigens and present them to T cells.

An “intracellular signaling domain,” as the term is used herein, refers to an intracellular portion of a molecule. The intracellular signaling domain generates a signal that promotes an immune effector function of the TFP containing cell, e.g., a TFP-expressing T cell. Examples of immune effector function, e.g., in a TFP-expressing T cell, include cytolytic activity and T helper cell activity, including the secretion of cytokines. In an embodiment, the intracellular signaling domain can comprise a primary intracellular signaling domain. Exemplary primary intracellular signaling domains include those derived from the molecules responsible for primary stimulation, or antigen dependent simulation. In an embodiment, the intracellular signaling domain can comprise a costimulatory intracellular domain. Exemplary costimulatory intracellular signaling domains include those derived from molecules responsible for costimulatory signals, or antigen independent stimulation.

A primary intracellular signaling domain can comprise an ITAM (“immunoreceptor tyrosine-based activation motif”). Examples of ITAM containing primary cytoplasmic signaling sequences include, but are not limited to, those derived from CD3 zeta, FcR gamma, FcR beta, CD3 gamma, CD3 delta, CD3 epsilon, CD5, CD22, CD79a, CD79b, and CD66d DAP10 and DAP12.

The term “costimulatory molecule” refers to the cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are required for an efficient immune response. Costimulatory molecules include, but are not limited to an MHC class 1 molecule, BTLA and a Toll ligand receptor, as well as DAP10, DAP12, CD30, LIGHT, OX40, CD2, CD27, CD28, CDS, ICAM-1, LFA-1 (CD11a/CD18) and 4-1BB (CD137). A costimulatory intracellular signaling domain can be the intracellular portion of a costimulatory molecule. A costimulatory molecule can be represented in the following protein families: TNF receptor proteins, Immunoglobulin-like proteins, cytokine receptors, integrins, signaling lymphocytic activation molecules (SLAM proteins), and activating NK cell receptors. Examples of such molecules include CD27, CD28, 4-1BB (CD137), OX40, GITR, CD30, CD40, ICOS, BAFFR, HVEM, lymphocyte function-associated antigen-1 (LFA-1), CD2, CD7, LIGHT, NKG2C, SLAMF7, NKp80, CD160, B7-H3, and a ligand that specifically binds with CD83, and the like. The intracellular signaling domain can comprise the entire intracellular portion, or the entire native intracellular signaling domain, of the molecule from which it is derived, or a functional fragment thereof. The term “4-1BB” refers to a member of the TNFR superfamily with an amino acid sequence provided as GenBank Acc. No. AAA62478.2, or the equivalent residues from a non-human species, e.g., mouse, rodent, monkey, ape and the like; and a “4-1BB costimulatory domain” is defined as amino acid residues 214-255 of GenBank Acc. No. AAA62478.2, or equivalent residues from non-human species, e.g., mouse, rodent, monkey, ape and the like.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain one or more introns.

The term “effective amount” or “therapeutically effective amount” are used interchangeably herein, and refer to an amount of a compound, formulation, material, or composition, as described herein effective to achieve a particular biological or therapeutic result.

The term “endogenous” refers to any material from or produced inside an organism, cell, tissue or system.

The term “exogenous” refers to any material introduced from or produced outside an organism, cell, tissue or system.

The term “expression” refers to the transcription and/or translation of a particular nucleotide sequence driven by a promoter.

The term “transfer vector” refers to a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “transfer vector” includes an autonomously replicating plasmid or a virus. The term should also be construed to further include non-plasmid and non-viral compounds which facilitate transfer of nucleic acid into cells, such as, for example, a polylysine compound, liposome, and the like. Examples of viral transfer vectors include, but are not limited to, adenoviral vectors, adeno-associated virus vectors, retroviral vectors, lentiviral vectors, and the like.

The term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses.

The term “lentiviral vector” refers to a vector derived from at least a portion of a lentivirus genome, including especially a self-inactivating lentiviral vector as provided in Milone et al., Mol. Ther. 17(8): 1453-1464 (2009). Other examples of lentivirus vectors that may be used in the clinic, include but are not limited to, e.g., the LENTIVECTOR™ gene delivery technology from Oxford BioMedica, the LENTIMAX™ vector system from Lentigen, and the like. Nonclinical types of lentiviral vectors are also available and would be known to one skilled in the art.

The term “homologous” or “identity” refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous or identical at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Humanized” forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. For the most part, humanized antibodies and antibody fragments thereof are human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

“Human” or “fully human” refers to an immunoglobulin, such as an antibody or antibody fragment, where the whole molecule is of human origin or consists of an amino acid sequence identical to a human form of the antibody or immunoglobulin.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “operably linked” or “transcriptional control” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

The term “parenteral” administration of an immunogenic composition includes, e.g., subcutaneous (s.c.), intravenous (i.v.), intramuscular (i.m.), or intrasternal injection, intratumoral, or infusion techniques.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

The term “promoter” refers to a DNA sequence recognized by the transcription machinery of the cell, or introduced synthetic machinery, required to initiate the specific transcription of a polynucleotide sequence.

The term “promoter/regulatory sequence” refers to a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

The term “constitutive” promoter refers to a nucleotide sequence which, when operably linked with a polynucleotide which encodes or specifies a gene product, causes the gene product to be produced in a cell under most or all physiological conditions of the cell.

The term “inducible” promoter refers to a nucleotide sequence which, when operably linked with a polynucleotide which encodes or specifies a gene product, causes the gene product to be produced in a cell substantially only when an inducer which corresponds to the promoter is present in the cell.

The term “tissue-specific” promoter refers to a nucleotide sequence which, when operably linked with a polynucleotide encodes or specified by a gene, causes the gene product to be produced in a cell substantially only if the cell is a cell of the tissue type corresponding to the promoter.

The terms “linker” and “flexible polypeptide linker” as used in the context of a scFv refers to a peptide linker that consists of amino acids such as glycine and/or serine residues used alone or in combination, to link variable heavy and variable light chain regions together. In one embodiment, the flexible polypeptide linker is a Gly/Ser linker and comprises the amino acid sequence (Gly-Gly-Gly-Ser)ₙ, where n is a positive integer equal to or greater than 1. For example, n=1, n=2, n=3, n=4, n=5, n=6, n=7, n=8, n=9 and n=10. In one embodiment, the flexible polypeptide linkers include, but are not limited to, (Gly₄Ser)₄ or (Gly₄Ser)₃. In another embodiment, the linkers include multiple repeats of (Gly₂Ser), (GlySer) or (Gly₃Ser). Also included within the scope of the invention are linkers described in WO2012/138475 (incorporated herein by reference). In some instances, the linker sequence comprises a long linker (LL) sequence. In some instances, the long linker sequence comprises (G₄S)ₙ, wherein n=2 to 4. In some instances, the linker sequence comprises a short linker (SL) sequence. In some instances, the short linker sequence comprises (G₄S)ₙ, wherein n=1 to 3.

As used herein, a 5′ cap (also termed an RNA cap, an RNA 7-methylguanosine cap or an RNA m7G cap) is a modified guanine nucleotide that has been added to the “front” or 5′ end of a eukaryotic messenger RNA shortly after the start of transcription. The 5′ cap consists of a terminal group which is linked to the first transcribed nucleotide. Its presence is critical for recognition by the ribosome and protection from RNases. Cap addition is coupled to transcription, and occurs co-transcriptionally, such that each influences the other. Shortly after the start of transcription, the 5′ end of the mRNA being synthesized is bound by a cap-synthesizing complex associated with RNA polymerase. This enzymatic complex catalyzes the chemical reactions that are required for mRNA capping. Synthesis proceeds as a multi-step biochemical reaction. The capping moiety can be modified to modulate functionality of mRNA such as its stability or efficiency of translation.

As used herein, “in vitro transcribed RNA” refers to RNA, preferably mRNA, which has been synthesized in vitro. Generally, the in vitro transcribed RNA is generated from an in vitro transcription vector. The in vitro transcription vector comprises a template that is used to generate the in vitro transcribed RNA.

As used herein, a “poly(A)” is a series of adenosines attached by polyadenylation to the mRNA. In the preferred embodiment of a construct for transient expression, the polyA is between 50 and 5000, preferably greater than 64, more preferably greater than 100, most preferably greater than 300 or 400. Poly(A) sequences can be modified chemically or enzymatically to modulate mRNA functionality such as localization, stability or efficiency of translation.

As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. The 3′ poly(A) tail is a long sequence of adenine nucleotides (often several hundred) added to the pre-mRNA through the action of an enzyme, polyadenylate polymerase. In higher eukaryotes, the poly(A) tail is added onto transcripts that contain a specific sequence, the polyadenylation signal. The poly(A) tail and the protein bound to it aid in protecting mRNA from degradation by exonucleases. Polyadenylation is also important for transcription termination, export of the mRNA from the nucleus, and translation. Polyadenylation occurs in the nucleus immediately after transcription of DNA into RNA, but additionally can also occur later in the cytoplasm. After transcription has been terminated, the mRNA chain is cleaved through the action of an endonuclease complex associated with RNA polymerase. The cleavage site is usually characterized by the presence of the base sequence AAUAAA near the cleavage site. After the mRNA has been cleaved, adenosine residues are added to the free 3′ end at the cleavage site.

As used herein, “transient” refers to expression of a non-integrated transgene for a period of hours, days or weeks, wherein the period of time of expression is less than the period of time for expression of the gene if integrated into the genome or contained within a stable plasmid replicon in the host cell.

The term “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the membrane of a cell.

The term “subject” is intended to include living organisms in which an immune response can be elicited (e.g., mammals, human).

The term, a “substantially purified” cell refers to a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some aspects, the cells are cultured in vitro. In other aspects, the cells are not cultured in vitro.

The term “therapeutic” as used herein means a treatment. A therapeutic effect is obtained by reduction, suppression, remission, or eradication of a disease state.

The term “prophylaxis” as used herein means the prevention of or protective treatment for a disease or disease state.

In the context of the present invention, “tumor antigen” or “hyperproliferative disorder antigen” or “antigen associated with a hyperproliferative disorder” refers to antigens that are common to specific hyperproliferative disorders. In certain aspects, the hyperproliferative disorder antigens of the present invention are derived from, cancers including but not limited to primary or metastatic melanoma

mesothelioma, renal cell carcinoma, stomach cancer, breast cancer, lung cancer, ovarian cancer, prostate cancer, colon cancer, cervical cancer, brain cancer, liver cancer, pancreatic cancer, kidney, endometrial, and stomach cancer.

In some instances, the disease is a cancer selected from the group consisting of mesothelioma, papillary serous ovarian adenocarcinoma, clear cell ovarian carcinoma, mixed Mullerian ovarian carcinoma, endometroid mucinous ovarian carcinoma, malignant pleural disease, pancreatic adenocarcinoma, ductal pancreatic adenocarcinoma, uterine serous carcinoma, lung adenocarcinoma, extrahepatic bile duct carcinoma, gastric adenocarcinoma, esophageal adenocarcinoma, colorectal adenocarcinoma, breast adenocarcinoma, a disease associated with mesothelin expression, and combinations thereof, a disease associated with mesothelin expression, and combinations thereof.

The term “transfected” or “transformed” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “specifically binds,” refers to an antibody, an antibody fragment or a specific ligand, which recognizes and binds a cognate binding partner (e.g., mesothelin) present in a sample, but which does not necessarily and substantially recognize or bind other molecules in the sample.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98% or 99% identity, and includes subranges such as 96-99%, 96-98%, 96-97%, 97-99%, 97-98% and 98-99% identity. This applies regardless of the breadth of the range.

### DESCRIPTION

Provided herein are compositions of matter and methods of use for the treatment of a disease such as cancer, using T cell receptor (TCR) fusion proteins. As used herein, a “T cell receptor (TCR) fusion protein” or “TFP” includes a recombinant polypeptide derived from the various polypeptides comprising the TCR that is generally capable of i) binding to a surface antigen on target cells and ii) interacting with other polypeptide components of the intact TCR complex, typically when co-located in or on the surface of a T cell. As provided herein, TFPs provide substantial benefits as compared to Chimeric Antigen Receptors. The term “Chimeric Antigen Receptor” or alternatively a “CAR” refers to a recombinant polypeptide comprising an extracellular antigen binding domain in the form of a scFv, a transmembrane domain, and cytoplasmic signaling domains (also referred to herein as “an intracellular signaling domains”) comprising a functional signaling domain derived from a stimulatory molecule as defined below. Generally, the central intracellular signaling domain of a CAR is derived from the CD3 zeta chain that is normally found associated with the TCR complex. The CD3 zeta signaling domain can be fused with one or more functional signaling domains derived from at least one co-stimulatory molecule such as 4-1BB (i.e., CD137), CD27 and/or CD28.

### T Cell Receptor (TCR) Fusion Proteins (TFP)

The present invention encompasses recombinant DNA constructs encoding TFPs, wherein the TFP comprises an antibody fragment that binds specifically to mesothelin, e.g., human mesothelin, wherein the sequence of the antibody fragment is contiguous with and in the same reading frame as a nucleic acid sequence encoding a TCR subunit or portion thereof. The TFPs provided herein are able to associate with one or more endogenous (or alternatively, one or more exogenous, or a combination of endogenous and exogenous) TCR subunits in order to form a functional TCR complex.

In one aspect, the TFP of the invention comprises a target-specific binding element otherwise referred to as an antigen binding domain. The choice of moiety depends upon the type and number of target antigen that define the surface of a target cell. For example, the antigen binding domain may be chosen to recognize a target antigen that acts as a cell surface marker on target cells associated with a particular disease state. Thus, examples of cell surface markers that may act as target antigens for the antigen binding domain in a TFP of the invention include those associated with viral, bacterial and parasitic infections; autoimmune diseases; and cancerous diseases (e.g., malignant diseases).

In one aspect, the TFP-mediated T cell response can be directed to an antigen of interest by way of engineering an antigen-binding domain into the TFP that specifically binds a desired antigen.

In one aspect, the portion of the TFP comprising the antigen binding domain comprises an antigen binding domain that targets mesothelin. In one aspect, the antigen binding domain targets human mesothelin.

The antigen binding domain can be any domain that binds to the antigen including but not limited to a monoclonal antibody, a polyclonal antibody, a recombinant antibody, a human antibody, a humanized antibody, and a functional fragment thereof, including but not limited to a single-domain antibody such as a heavy chain variable domain (V_{H}), a light chain variable domain (V_{L}) and a variable domain (V_{HH}) of a camelid derived nanobody, and to an alternative scaffold known in the art to function as antigen binding domain, such as a recombinant fibronectin domain, anticalin, DARPIN and the like. Likewise a natural or synthetic ligand specifically recognizing and binding the target antigen can be used as antigen binding domain for the TFP. In some instances, it is beneficial for the antigen binding domain to be derived from the same species in which the TFP will ultimately be used in. For example, for use in humans, it may be beneficial for the antigen binding domain of the TFP to comprise human or humanized residues for the antigen binding domain of an antibody or antibody fragment.

Thus, in one aspect, the antigen-binding domain comprises a humanized or human antibody or an antibody fragment, or a murine antibody or antibody fragment. In one embodiment, the humanized or human anti-mesothelin binding domain comprises one or more (e.g., all three) light chain complementary determining region 1 (LC CDR1), light chain complementary determining region 2 (LC CDR2), and light chain complementary determining region 3 (LC CDR3) of a humanized or human anti-mesothelin binding domain described herein, and/or one or more (e.g., all three) heavy chain complementary determining region 1 (HC CDR1), heavy chain complementary determining region 2 (HC CDR2), and heavy chain complementary determining region 3 (HC CDR3) of a humanized or human anti-mesothelin binding domain described herein, e.g., a humanized or human anti-mesothelin binding domain comprising one or more, e.g., all three, LC CDRs and one or more, e.g., all three, HC CDRs. In one embodiment, the humanized or human anti-mesothelin binding domain comprises one or more (e.g., all three) heavy chain complementary determining region 1 (HC CDR1), heavy chain complementary determining region 2 (HC CDR2), and heavy chain complementary determining region 3 (HC CDR3) of a humanized or human anti-mesothelin binding domain described herein, e.g., the humanized or human anti-mesothelin binding domain has two variable heavy chain regions, each comprising a HC CDR1, a HC CDR2 and a HC CDR3 described herein. In one embodiment, the humanized or human anti-mesothelin binding domain comprises a humanized or human light chain variable region described herein and/or a humanized or human heavy chain variable region described herein. In one embodiment, the humanized or human anti-mesothelin binding domain comprises a humanized heavy chain variable region described herein, e.g., at least two humanized or human heavy chain variable regions described herein. In one embodiment, the anti-mesothelin binding domain is a scFv comprising a light chain and a heavy chain of an amino acid sequence provided herein. In an embodiment, the anti-mesothelin binding domain (e.g., a scFv) comprises: a light chain variable region comprising an amino acid sequence having at least one, two or three modifications (e.g., substitutions) but not more than 30, 20 or 10 modifications (e.g., substitutions) of an amino acid sequence of a light chain variable region provided herein, or a sequence with 95-99% identity with an amino acid sequence provided herein; and/or a heavy chain variable region comprising an amino acid sequence having at least one, two or three modifications (e.g., substitutions) but not more than 30, 20 or 10 modifications (e.g., substitutions) of an amino acid sequence of a heavy chain variable region provided herein, or a sequence with 95-99% identity to an amino acid sequence provided herein. In one embodiment, the humanized or human anti-mesothelin binding domain is a scFv, and a light chain variable region comprising an amino acid sequence described herein, is attached to a heavy chain variable region comprising an amino acid sequence described herein, via a linker, e.g., a linker described herein. In one embodiment, the humanized anti-mesothelin binding domain includes a (Gly₄-Ser)ₙ linker, wherein n is 1, 2, 3, 4, 5, or 6, preferably 3 or 4. The light chain variable region and heavy chain variable region of a scFv can be, e.g., in any of the following orientations: light chain variable region-linker-heavy chain variable region or heavy chain variable region-linker-light chain variable region. In some instances, the linker sequence comprises a long linker (LL) sequence. In some instances, the long linker sequence comprises (G₄S)ₙ, wherein n=2 to 4. In some instances, the linker sequence comprises a short linker (SL) sequence. In some instances, the short linker sequence comprises (G₄S)ₙ, wherein n=1 to 3.

In some aspects, a non-human antibody is humanized, where specific sequences or regions of the antibody are modified to increase similarity to an antibody naturally produced in a human or fragment thereof. In one aspect, the antigen binding domain is humanized.

A humanized antibody can be produced using a variety of techniques known in the art, including but not limited to, CDR-grafting (see, e.g., European Patent No. EP 239,400; International Publication No. WO 91/09967; and U.S. Pat. Nos. 5,225,539, 5,530,101, and 5,585,089, each of which is incorporated herein in its entirety by reference), veneering or resurfacing (see, e.g., European Patent Nos. EP 592,106 and EP 519,596; Padlan, 1991, Molecular Immunology, 28(4/5):489-498; Studnicka et al., 1994, Protein Engineering, 7(6):805-814; and Roguska et al., 1994, PNAS, 91:969-973, each of which is incorporated herein by its entirety by reference), chain shuffling (see, e.g., U.S. Pat. No. 5,565,332, which is incorporated herein in its entirety by reference), and techniques disclosed in, e.g., U.S. Patent Application Publication No. US2005/0042664, U.S. Patent Application Publication No. US2005/0048617, U.S. Pat. Nos. 6,407,213, 5,766,886, International Publication No. WO 9317105, Tan et al., J. Immunol., 169:1119-25 (2002), Caldas et al., Protein Eng., 13(5):353-60 (2000), Morea et al., Methods, 20(3):267-79 (2000), Baca et al., J. Biol. Chem., 272(16):10678-84 (1997), Roguska et al., Protein Eng., 9(10):895-904 (1996), Couto et al., Cancer Res., 55 (23 Supp):5973s-5977s (1995), Couto et al., Cancer Res., 55(8):1717-22 (1995), Sandhu J S, Gene, 150(2):409-10 (1994), and Pedersen et al., J. Mol. Biol., 235(3):959-73 (1994), each of which is incorporated herein in its entirety by reference. Often, framework residues in the framework regions will be substituted with the corresponding residue from the CDR donor antibody to alter, for example improve, antigen binding. These framework substitutions are identified by methods well-known in the art, e.g., by modeling of the interactions of the CDR and framework residues to identify framework residues important for antigen binding and sequence comparison to identify unusual framework residues at particular positions (see, e.g., Queen et al., U.S. Pat. No. 5,585,089; and Riechmann et al., 1988, Nature, 332:323, which are incorporated herein by reference in their entireties.)

A humanized antibody or antibody fragment has one or more amino acid residues remaining in it from a source which is nonhuman. These nonhuman amino acid residues are often referred to as “import” residues, which are typically taken from an “import” variable domain. As provided herein, humanized antibodies or antibody fragments comprise one or more CDRs from nonhuman immunoglobulin molecules and framework regions wherein the amino acid residues comprising the framework are derived completely or mostly from human germline. Multiple techniques for humanization of antibodies or antibody fragments are well-known in the art and can essentially be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody, i.e., CDR-grafting (EP 239,400; PCT Publication No. WO 91/09967; and U.S. Pat. Nos. 4,816,567; 6,331,415; 5,225,539; 5,530,101; 5,585,089; 6,548,640, the contents of which are incorporated herein by reference in their entirety). In such humanized antibodies and antibody fragments, substantially less than an intact human variable domain has been substituted by the corresponding sequence from a nonhuman species. Humanized antibodies are often human antibodies in which some CDR residues and possibly some framework (FR) residues are substituted by residues from analogous sites in rodent antibodies. Humanization of antibodies and antibody fragments can also be achieved by veneering or resurfacing (EP 592,106; EP 519,596; Padlan, 1991, Molecular Immunology, 28(4/5):489-498; Studnicka et al., Protein Engineering, 7(6):805-814 (1994); and Roguska et al., PNAS, 91:969-973 (1994)) or chain shuffling (U.S. Pat. No. 5,565,332), the contents of which are incorporated herein by reference in their entirety.

The choice of human variable domains, both light and heavy, to be used in making the humanized antibodies is to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., J. Immunol., 151:2296 (1993); Chothia et al., J. Mol. Biol., 196:901 (1987), the contents of which are incorporated herein by reference herein in their entirety). Another method uses a particular framework derived from the consensus sequence of all human antibodies of a particular subgroup of light or heavy chains. The same framework may be used for several different humanized antibodies (see, e.g., Nicholson et al. Mol. Immun. 34 (16-17): 1157-1165 (1997); Carter et al., Proc. Natl. Acad. Sci. USA, 89:4285 (1992); Presta et al., J. Immunol., 151:2623 (1993), the contents of which are incorporated herein by reference herein in their entirety). In some embodiments, the framework region, e.g., all four framework regions, of the heavy chain variable region are derived from a V_{H}4-4-59 germline sequence. In one embodiment, the framework region can comprise, one, two, three, four or five modifications, e.g., substitutions, e.g., from the amino acid at the corresponding murine sequence. In one embodiment, the framework region, e.g., all four framework regions of the light chain variable region are derived from a VK3-1.25 germline sequence. In one embodiment, the framework region can comprise, one, two, three, four or five modifications, e.g., substitutions, e.g., from the amino acid at the corresponding murine sequence.

In some aspects, the portion of a TFP composition of the invention that comprises an antibody fragment is humanized with retention of high affinity for the target antigen and other favorable biological properties. According to one aspect of the invention, humanized antibodies and antibody fragments are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, e.g., the analysis of residues that influence the ability of the candidate immunoglobulin to bind the target antigen. In this way, FR residues can be selected and combined from the recipient and import sequences so that the desired antibody or antibody fragment characteristic, such as increased affinity for the target antigen, is achieved. In general, the CDR residues are directly and most substantially involved in influencing antigen binding.

A humanized antibody or antibody fragment may retain a similar antigenic specificity as the original antibody, e.g., in the present invention, the ability to bind human mesothelin. In some embodiments, a humanized antibody or antibody fragment may have improved affinity and/or specificity of binding to human mesothelin.

In one aspect, the anti-mesothelin binding domain is characterized by particular functional features or properties of an antibody or antibody fragment. For example, in one aspect, the portion of a TFP composition of the invention that comprises an antigen binding domain specifically binds human mesothelin. In one aspect, the antigen binding domain has the same or a similar binding specificity to human mesothelin as the FMC63 scFv described in Nicholson et al. Mol. Immun. 34 (16-17): 1157-1165 (1997). In one aspect, the invention relates to an antigen binding domain comprising an antibody or antibody fragment, wherein the antibody binding domain specifically binds to a mesothelin protein or fragment thereof, wherein the antibody or antibody fragment comprises a variable light chain and/or a variable heavy chain that includes an amino acid sequence provided herein. In certain aspects, the scFv is contiguous with and in the same reading frame as a leader sequence.

In one aspect, the anti-mesothelin binding domain is a fragment, e.g., a single chain variable fragment (scFv). In one aspect, the anti-mesothelin binding domain is a Fv, a Fab, a (Fab′)₂, or a bi-functional (e.g. bi-specific) hybrid antibody (e.g., Lanzavecchia et al., Eur. J. Immunol. 17, 105 (1987)). In one aspect, the antibodies and fragments thereof disclosed herein bind a mesothelin protein with wild-type or enhanced affinity.

Also provided herein are methods for obtaining an antibody antigen binding domain specific for a target antigen (e.g., mesothelin or any target antigen described elsewhere herein for targets of fusion moiety binding domains), the method comprising providing by way of addition, deletion, substitution or insertion of one or more amino acids in the amino acid sequence of a V_{H} domain set out herein a V_{H} domain which is an amino acid sequence variant of the V_{H} domain, optionally combining the V_{H} domain thus provided with one or more V_{L} domains, and testing the V_{H} domain or V_{H}/V_{L} combination or combinations to identify a specific binding member or an antibody antigen binding domain specific for a target antigen of interest (e.g., mesothelin) and optionally with one or more desired properties.

In some instances, V_{H} domains and scFvs can be prepared according to method known in the art (see, for example, Bird et al., (1988) Science 242:423-426 and Huston et al., (1988) Proc. Natl. Acad. Sci. USA 85:5879-5883). scFv molecules can be produced by linking V_{H} and V_{L} regions together using flexible polypeptide linkers. The scFv molecules comprise a linker (e.g., a Ser-Gly linker) with an optimized length and/or amino acid composition. The linker length can greatly affect how the variable regions of a scFv fold and interact. In fact, if a short polypeptide linker is employed (e.g., between 5-10 amino acids) intra-chain folding is prevented. Inter-chain folding is also required to bring the two variable regions together to form a functional epitope binding site. In some instances, the linker sequence comprises a long linker (LL) sequence. In some instances, the long linker sequence comprises (G₄S)ₙ, wherein n=2 to 4. In some instances, the linker sequence comprises a short linker (SL) sequence. In some instances, the short linker sequence comprises (G₄S)ₙ, wherein n=1 to 3. For examples of linker orientation and size see, e.g., Hollinger et al. 1993 Proc Natl Acad. Sci. U.S.A. 90:6444-6448, U.S. Pat. No. 7,695,936, U.S. Patent Application Publication Nos. 20050100543 and 20050175606, and PCT Publication Nos. WO2006/020258 and WO2007/024715, all of which are incorporated herein by reference.

A scFv can comprise a linker of about 10, 11, 12, 13, 14, 15 or greater than 15 residues between its V_{L} and V_{H} regions. The linker sequence may comprise any naturally occurring amino acid. In some embodiments, the linker sequence comprises amino acids glycine and serine. In another embodiment, the linker sequence comprises sets of glycine and serine repeats such as (Gly₄Ser)ₙ, where n is a positive integer equal to or greater than 1. In one embodiment, the linker can be (Gly₄Ser)₄ or (Gly₄Ser)₃. Variation in the linker length may retain or enhance activity, giving rise to superior efficacy in activity studies. In some instances, the linker sequence comprises a long linker (LL) sequence. In some instances, the long linker sequence comprises (G₄S)ₙ, wherein n=2 to 4. In some instances, the linker sequence comprises a short linker (SL) sequence. In some instances, the short linker sequence comprises (G₄S)ₙ, wherein n=1 to 3.

### Stability and Mutations

The stability of an anti-mesothelin binding domain, e.g., scFv molecules (e.g., soluble scFv) can be evaluated in reference to the biophysical properties (e.g., thermal stability) of a conventional control scFv molecule or a full length antibody. In one embodiment, the humanized or human scFv has a thermal stability that is greater than about 0.1, about 0.25, about 0.5, about 0.75, about 1, about 1.25, about 1.5, about 1.75, about 2, about 2.5, about 3, about 3.5, about 4, about 4.5, about 5, about 5.5, about 6, about 6.5, about 7, about 7.5, about 8, about 8.5, about 9, about 9.5, about 10 degrees, about 11 degrees, about 12 degrees, about 13 degrees, about 14 degrees, or about 15 degrees Celsius than a parent scFv in the described assays.

The improved thermal stability of the anti-mesothelin binding domain, e.g., scFv is subsequently conferred to the entire mesothelin-TFP construct, leading to improved therapeutic properties of the anti-mesothelin TFP construct. The thermal stability of the anti-mesothelin binding domain, e.g., scFv can be improved by at least about 2° C. or 3° C. as compared to a conventional antibody. In one embodiment, the anti-mesothelin binding domain, e.g., scFv has a 1° C. improved thermal stability as compared to a conventional antibody. In another embodiment, the anti-mesothelin binding domain, e.g., scFv has a 2° C. improved thermal stability as compared to a conventional antibody. In another embodiment, the scFv has a 4° C., 5° C., 6° C., 7° C., 8° C., 9° C., 10° C., 11° C., 12° C., 13° C., 14° C., or 15° C. improved thermal stability as compared to a conventional antibody. Comparisons can be made, for example, between the scFv molecules disclosed herein and scFv molecules or Fab fragments of an antibody from which the scFv V_{H} and V_{L} were derived. Thermal stability can be measured using methods known in the art. For example, in one embodiment, T_{M} can be measured. Methods for measuring T_{M} and other methods of determining protein stability are described below.

Mutations in scFv (arising through humanization or mutagenesis of the soluble scFv) alter the stability of the scFv and improve the overall stability of the scFv and the anti-mesothelin TFP construct. Stability of the humanized scFv is compared against the murine scFv using measurements such as T_{M}, temperature denaturation and temperature aggregation. In one embodiment, the anti-mesothelin binding domain, e.g., a scFv, comprises at least one mutation arising from the humanization process such that the mutated scFv confers improved stability to the anti-mesothelin TFP construct. In another embodiment, the anti-mesothelin binding domain, e.g., scFv comprises at least 1, 2, 3, 4, 5, 6, 7, 8, 9, 10 mutations arising from the humanization process such that the mutated scFv confers improved stability to the mesothelin-TFP construct.

In one aspect, the antigen binding domain of the TFP comprises an amino acid sequence that is homologous to an antigen binding domain amino acid sequence described herein, and the antigen binding domain retains the desired functional properties of the anti-mesothelin antibody fragments described herein. In one specific aspect, the TFP composition of the invention comprises an antibody fragment. In a further aspect, that antibody fragment comprises a scFv.

In various aspects, the antigen binding domain of the TFP is engineered by modifying one or more amino acids within one or both variable regions (e.g., V_{H} and/or V_{L}), for example within one or more CDR regions and/or within one or more framework regions. In one specific aspect, the TFP composition of the invention comprises an antibody fragment. In a further aspect, that antibody fragment comprises a scFv.

It will be understood by one of ordinary skill in the art that the antibody or antibody fragment of the invention may further be modified such that they vary in amino acid sequence (e.g., from wild-type), but not in desired activity. For example, additional nucleotide substitutions leading to amino acid substitutions at “non-essential” amino acid residues may be made to the protein. For example, a nonessential amino acid residue in a molecule may be replaced with another amino acid residue from the same side chain family. In another embodiment, a string of amino acids can be replaced with a structurally similar string that differs in order and/or composition of side chain family members, e.g., a conservative substitution, in which an amino acid residue is replaced with an amino acid residue having a similar side chain, may be made.

Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Percent identity in the context of two or more nucleic acids or polypeptide sequences refers to two or more sequences that are the same. Two sequences are “substantially identical” if two sequences have a specified percentage of amino acid residues or nucleotides that are the same (e.g., 60% identity, optionally 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more preferably over a region that is 100 to 500 or 1000 or more nucleotides (or 20, 50, 200 or more amino acids) in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Methods of alignment of sequences for comparison are well known in the art. Optimal alignment of sequences for comparison can be conducted, e.g., by the local homology algorithm of Smith and Waterman, (1970) Adv. Appl. Math. 2:482c, by the homology alignment algorithm of Needleman and Wunsch, (1970) J. Mol. Biol. 48:443, by the search for similarity method of Pearson and Lipman, (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Brent et al., (2003) Current Protocols in Molecular Biology). Two examples of algorithms that are suitable for determining percent sequence identity and sequence similarity are the BLAST and BLAST 2.0 algorithms, which are described in Altschul et al., (1977) Nuc. Acids Res. 25:3389-3402; and Altschul et al., (1990) J. Mol. Biol. 215:403-410, respectively. Software for performing BLAST analyses is publicly available through the National Center for Biotechnology Information.

In one aspect, the present invention contemplates modifications of the starting antibody or fragment (e.g., scFv) amino acid sequence that generate functionally equivalent molecules. For example, the V_{H} or V_{L} of an anti-mesothelin binding domain, e.g., scFv, comprised in the TFP can be modified to retain at least about 70%, 71%. 72%. 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity of the starting V_{H} or V_{L} framework region of the anti-mesothelin binding domain, e.g., scFv. The present invention contemplates modifications of the entire TFP construct, e.g., modifications in one or more amino acid sequences of the various domains of the TFP construct in order to generate functionally equivalent molecules. The TFP construct can be modified to retain at least about 70%, 71%. 72%. 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity of the starting TFP construct.

### Extracellular Domain

The extracellular domain may be derived either from a natural or from a recombinant source. Where the source is natural, the domain may be derived from any protein, but in particular a membrane-bound or transmembrane protein. In one aspect the extracellular domain is capable of associating with the transmembrane domain. An extracellular domain of particular use in this invention may include at least the extracellular region(s) of e.g., the alpha, beta or zeta chain of the T cell receptor, or CD3 epsilon, CD3 gamma, or CD3 delta, or in alternative embodiments, CD28, CD45, CD4, CD5, CD8, CD9, CD16, CD22, CD33, CD37, CD64, CD80, CD86, CD134, CD137, CD154.

### Transmembrane Domain

In general, a TFP sequence contains an extracellular domain and a transmembrane domain encoded by a single genomic sequence. In alternative embodiments, a TFP can be designed to comprise a transmembrane domain that is heterologous to the extracellular domain of the TFP. A transmembrane domain can include one or more additional amino acids adjacent to the transmembrane region, e.g., one or more amino acid associated with the extracellular region of the protein from which the transmembrane was derived (e.g., 1, 2, 3, 4, 5, 6, 7, 8, 9, 10 or up to 15 amino acids of the extracellular region) and/or one or more additional amino acids associated with the intracellular region of the protein from which the transmembrane protein is derived (e.g., 1, 2, 3, 4, 5, 6, 7, 8, 9, 10 or up to 15 amino acids of the intracellular region). In one aspect, the transmembrane domain is one that is associated with one of the other domains of the TFP is used. In some instances, the transmembrane domain can be selected or modified by amino acid substitution to avoid binding of such domains to the transmembrane domains of the same or different surface membrane proteins, e.g., to minimize interactions with other members of the receptor complex. In one aspect, the transmembrane domain is capable of homodimerization with another TFP on the TFP-T cell surface. In a different aspect the amino acid sequence of the transmembrane domain may be modified or substituted so as to minimize interactions with the binding domains of the native binding partner present in the same TFP.

The transmembrane domain may be derived either from a natural or from a recombinant source. Where the source is natural, the domain may be derived from any membrane-bound or transmembrane protein. In one aspect the transmembrane domain is capable of signaling to the intracellular domain(s) whenever the TFP has bound to a target. A transmembrane domain of particular use in this invention may include at least the transmembrane region(s) of e.g., the alpha, beta or zeta chain of the T cell receptor, CD28, CD3 epsilon, CD45, CD4, CD5, CD8, CD9, CD16, CD22, CD33, CD37, CD64, CD80, CD86, CD134, CD137, CD154.

In some instances, the transmembrane domain can be attached to the extracellular region of the TFP, e.g., the antigen binding domain of the TFP, via a hinge, e.g., a hinge from a human protein. For example, in one embodiment, the hinge can be a human immunoglobulin (Ig) hinge, e.g., an IgG4 hinge, or a CD8a hinge.

### Linkers

Optionally, a short oligo- or polypeptide linker, between 2 and 10 amino acids in length may form the linkage between the transmembrane domain and the cytoplasmic region of the TFP. A glycine-serine doublet provides a particularly suitable linker. For example, in one aspect, the linker comprises the amino acid sequence of GGGGSGGGGS (SEQ ID NO. 53). In some embodiments, the linker is encoded by a nucleotide sequence of

| | |
|---|---|
| GGTGGCGGAGGTTCTGGAGGTGGAGGTTCC. | (SEQ ID NO. 54) |

### Cytoplasmic Domain

The cytoplasmic domain of the TFP can include an intracellular signaling domain, if the TFP contains CD3 gamma, delta or epsilon polypeptides; TCR alpha and TCR beta subunits are generally lacking in a signaling domain. An intracellular signaling domain is generally responsible for activation of at least one of the normal effector functions of the immune cell in which the TFP has been introduced. The term “effector function” refers to a specialized function of a cell. Effector function of a T cell, for example, may be cytolytic activity or helper activity including the secretion of cytokines. Thus the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

Examples of intracellular signaling domains for use in the TFP of the invention include the cytoplasmic sequences of the T cell receptor (TCR) and co-receptors that act in concert to initiate signal transduction following antigen receptor engagement, as well as any derivative or variant of these sequences and any recombinant sequence that has the same functional capability.

It is known that signals generated through the TCR alone are insufficient for full activation of naive T cells and that a secondary and/or costimulatory signal is required. Thus, naive T cell activation can be said to be mediated by two distinct classes of cytoplasmic signaling sequences: those that initiate antigen-dependent primary activation through the TCR (primary intracellular signaling domains) and those that act in an antigen-independent manner to provide a secondary or costimulatory signal (secondary cytoplasmic domain, e.g., a costimulatory domain).

A primary signaling domain regulates primary activation of the TCR complex either in a stimulatory way, or in an inhibitory way. Primary intracellular signaling domains that act in a stimulatory manner may contain signaling motifs which are known as immunoreceptor tyrosine-based activation motifs (ITAMs).

Examples of ITAMs containing primary intracellular signaling domains that are of particular use in the invention include those of CD3 zeta, FcR gamma, FcR beta, CD3 gamma, CD3 delta, CD3 epsilon, CD5, CD22, CD79a, CD79b, and CD66d. In one embodiment, a TFP of the invention comprises an intracellular signaling domain, e.g., a primary signaling domain of CD3-epsilon. In one embodiment, a primary signaling domain comprises a modified ITAM domain, e.g., a mutated ITAM domain which has altered (e.g., increased or decreased) activity as compared to the native ITAM domain. In one embodiment, a primary signaling domain comprises a modified ITAM-containing primary intracellular signaling domain, e.g., an optimized and/or truncated ITAM-containing primary intracellular signaling domain. In an embodiment, a primary signaling domain comprises one, two, three, four or more ITAM motifs.

The intracellular signaling domain of the TFP can comprise the CD3 zeta signaling domain by itself or it can be combined with any other desired intracellular signaling domain(s) useful in the context of a TFP of the invention. For example, the intracellular signaling domain of the TFP can comprise a CD3 epsilon chain portion and a costimulatory signaling domain. The costimulatory signaling domain refers to a portion of the TFP comprising the intracellular domain of a costimulatory molecule. A costimulatory molecule is a cell surface molecule other than an antigen receptor or its ligands that is required for an efficient response of lymphocytes to an antigen. Examples of such molecules include CD27, CD28, 4-1BB (CD137), OX40, DAP10, DAP12, CD30, CD40, PD1, ICOS, lymphocyte function-associated antigen-1 (LFA-1), CD2, CD7, LIGHT, NKG2C, B7-H3, and a ligand that specifically binds with CD83, and the like. For example, CD27 costimulation has been demonstrated to enhance expansion, effector function, and survival of human TFP-T cells in vitro and augments human T cell persistence and antitumor activity in vivo (Song et al. Blood. 2012; 119(3):696-706).

The intracellular signaling sequences within the cytoplasmic portion of the TFP of the invention may be linked to each other in a random or specified order. Optionally, a short oligo- or polypeptide linker, for example, between 2 and 10 amino acids (e.g., 2, 3, 4, 5, 6, 7, 8, 9, or 10 amino acids) in length may form the linkage between intracellular signaling sequences.

In one embodiment, a glycine-serine doublet can be used as a suitable linker. In one embodiment, a single amino acid, e.g., an alanine, a glycine, can be used as a suitable linker.

In one aspect, the TFP-expressing cell described herein can further comprise a second TFP, e.g., a second TFP that includes a different antigen binding domain, e.g., to the same target (mesothelin) or a different target (e.g., CD123). In one embodiment, when the TFP-expressing cell comprises two or more different TFPs, the antigen binding domains of the different TFPs can be such that the antigen binding domains do not interact with one another. For example, a cell expressing a first and second TFP can have an antigen binding domain of the first TFP, e.g., as a fragment, e.g., a scFv, that does not associate with the antigen binding domain of the second TFP, e.g., the antigen binding domain of the second TFP is a V_{HH}. In one embodiment, the antigen binding domain is SD1 (SEQ ID NO:53), SD4 (SEQ ID NO:54), or SD6 (SEQ ID NO:55)

In another aspect, the TFP-expressing cell described herein can further express another agent, e.g., an agent which enhances the activity of a TFP-expressing cell. For example, in one embodiment, the agent can be an agent which inhibits an inhibitory molecule. Inhibitory molecules, e.g., PD1, can, in some embodiments, decrease the ability of a TFP-expressing cell to mount an immune effector response. Examples of inhibitory molecules include PD1, PD-L1, CTLA4, TIM3, LAG3, VISTA, BTLA, TIGIT, LAIR1, CD160, 2B4 and TGFR beta. In one embodiment, the agent that inhibits an inhibitory molecule comprises a first polypeptide, e.g., an inhibitory molecule, associated with a second polypeptide that provides a positive signal to the cell, e.g., an intracellular signaling domain described herein. In one embodiment, the agent comprises a first polypeptide, e.g., of an inhibitory molecule such as PD1, LAG3, CTLA4, CD160, BTLA, LAIR1, TIM3, 2B4 and TIGIT, or a fragment of any of these (e.g., at least a portion of an extracellular domain of any of these), and a second polypeptide which is an intracellular signaling domain described herein (e.g., comprising a costimulatory domain (e.g., 4-1BB, CD27 or CD28, e.g., as described herein) and/or a primary signaling domain (e.g., a CD3 zeta signaling domain described herein). In one embodiment, the agent comprises a first polypeptide of PD1 or a fragment thereof (e.g., at least a portion of an extracellular domain of PD1), and a second polypeptide of an intracellular signaling domain described herein (e.g., a CD28 signaling domain described herein and/or a CD3 zeta signaling domain described herein). PD1 is an inhibitory member of the CD28 family of receptors that also includes CD28, CTLA-4, ICOS, and BTLA. PD-1 is expressed on activated B cells, T cells and myeloid cells (Agata et al. 1996 Int. Immunol 8:765-75). Two ligands for PD1, PD-L1 and PD-L2 have been shown to downregulate T cell activation upon binding to PD1 (Freeman et al. 2000 J Exp Med 192:1027-34; Latchman et al. 2001 Nat Immunol 2:261-8; Carter et al. 2002 Eur J Immunol 32:634-43). PD-L1 is abundant in human cancers (Dong et al. 2003 J Mol Med 81:281-7; Blank et al. 2005 Cancer Immunol. Immunother 54:307-314; Konishi et al. 2004 Clin Cancer Res 10:5094). Immune suppression can be reversed by inhibiting the local interaction of PD1 with PD-L1.

In one embodiment, the agent comprises the extracellular domain (ECD) of an inhibitory molecule, e.g., Programmed Death 1 (PD1) can be fused to a transmembrane domain and optionally an intracellular signaling domain such as 41BB and CD3 zeta (also referred to herein as a PD1 TFP). In one embodiment, the PD1 TFP, when used in combinations with an anti-mesothelin TFP described herein, improves the persistence of the T cell. In one embodiment, the TFP is a PD1 TFP comprising the extracellular domain of PD 1. Alternatively, provided are TFPs containing an antibody or antibody fragment such as a scFv that specifically binds to the Programmed Death-Ligand 1 (PD-L1) or Programmed Death-Ligand 2 (PD-L2).

In another aspect, the present invention provides a population of TFP-expressing T cells, e.g., TFP-T cells. In some embodiments, the population of TFP-expressing T cells comprises a mixture of cells expressing different TFPs. For example, in one embodiment, the population of TFP-T cells can include a first cell expressing a TFP having an anti-mesothelin binding domain described herein, and a second cell expressing a TFP having a different anti-mesothelin binding domain, e.g., an anti-mesothelin binding domain described herein that differs from the anti-mesothelin binding domain in the TFP expressed by the first cell. As another example, the population of TFP-expressing cells can include a first cell expressing a TFP that includes an anti-mesothelin binding domain, e.g., as described herein, and a second cell expressing a TFP that includes an antigen binding domain to a target other than mesothelin (e.g., another tumor-associated antigen).

In another aspect, the present invention provides a population of cells wherein at least one cell in the population expresses a TFP having an anti-mesothelin domain described herein, and a second cell expressing another agent, e.g., an agent which enhances the activity of a TFP-expressing cell. For example, in one embodiment, the agent can be an agent which inhibits an inhibitory molecule. Inhibitory molecules, e.g., can, in some embodiments, decrease the ability of a TFP-expressing cell to mount an immune effector response. Examples of inhibitory molecules include PD1, PD-L1, PD-L2, CTLA4, TIM3, LAG3, VISTA, BTLA, TIGIT, LAIR1, CD160, 2B4 and TGFR beta. In one embodiment, the agent that inhibits an inhibitory molecule comprises a first polypeptide, e.g., an inhibitory molecule, associated with a second polypeptide that provides a positive signal to the cell, e.g., an intracellular signaling domain described herein.

Disclosed herein are methods for producing in vitro transcribed RNA encoding TFPs. The present invention also includes a TFP encoding RNA construct that can be directly transfected into a cell. A method for generating mRNA for use in transfection can involve in vitro transcription (IVT) of a template with specially designed primers, followed by polyA addition, to produce a construct containing 3′ and 5′ untranslated sequence (“UTR”), a 5′ cap and/or Internal Ribosome Entry Site (IRES), the nucleic acid to be expressed, and a polyA tail, typically 50-2000 bases in length. RNA so produced can efficiently transfect different kinds of cells. In one aspect, the template includes sequences for the TFP.

In one aspect, the anti-mesothelin TFP is encoded by a messenger RNA (mRNA). In one aspect the mRNA encoding the anti-mesothelin TFP is introduced into a T cell for production of a TFP-T cell. In one embodiment, the in vitro transcribed RNA TFP can be introduced to a cell as a form of transient transfection. The RNA is produced by in vitro transcription using a polymerase chain reaction (PCR)-generated template. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA. The desired template for in vitro transcription is a TFP of the present invention. In one embodiment, the DNA to be used for PCR contains an open reading frame. The DNA can be from a naturally occurring DNA sequence from the genome of an organism. In one embodiment, the nucleic acid can include some or all of the 5′ and/or 3′ untranslated regions (UTRs). The nucleic acid can include exons and introns. In one embodiment, the DNA to be used for PCR is a human nucleic acid sequence. In another embodiment, the DNA to be used for PCR is a human nucleic acid sequence including the 5′ and 3′ UTRs. The DNA can alternatively be an artificial DNA sequence that is not normally expressed in a naturally occurring organism. An exemplary artificial DNA sequence is one that contains portions of genes that are ligated together to form an open reading frame that encodes a fusion protein. The portions of DNA that are ligated together can be from a single organism or from more than one organism.

PCR is used to generate a template for in vitro transcription of mRNA which is used for transfection. Methods for performing PCR are well known in the art. Primers for use in PCR are designed to have regions that are substantially complementary to regions of the DNA to be used as a template for the PCR. “Substantially complementary,” as used herein, refers to sequences of nucleotides where a majority or all of the bases in the primer sequence are complementary, or one or more bases are non-complementary, or mismatched. Substantially complementary sequences are able to anneal or hybridize with the intended DNA target under annealing conditions used for PCR. The primers can be designed to be substantially complementary to any portion of the DNA template. For example, the primers can be designed to amplify the portion of a nucleic acid that is normally transcribed in cells (the open reading frame), including 5′ and 3′ UTRs. The primers can also be designed to amplify a portion of a nucleic acid that encodes a particular domain of interest. In one embodiment, the primers are designed to amplify the coding region of a human cDNA, including all or portions of the 5′ and 3′ UTRs. Primers useful for PCR can be generated by synthetic methods that are well known in the art. “Forward primers” are primers that contain a region of nucleotides that are substantially complementary to nucleotides on the DNA template that are upstream of the DNA sequence that is to be amplified. “Upstream” is used herein to refer to a location 5, to the DNA sequence to be amplified relative to the coding strand. “Reverse primers” are primers that contain a region of nucleotides that are substantially complementary to a double-stranded DNA template that are downstream of the DNA sequence that is to be amplified. “Downstream” is used herein to refer to a location 3′ to the DNA sequence to be amplified relative to the coding strand.

Any DNA polymerase useful for PCR can be used in the methods disclosed herein. The reagents and polymerase are commercially available from a number of sources.

Chemical structures with the ability to promote stability and/or translation efficiency may also be used. The RNA preferably has 5′ and 3′ UTRs. In one embodiment, the 5′ UTR is between one and 3,000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the nucleic acid of interest. Alternatively, UTR sequences that are not endogenous to the nucleic acid of interest can be added by incorporating the UTR sequences into the forward and reverse primers or by any other modifications of the template. The use of UTR sequences that are not endogenous to the nucleic acid of interest can be useful for modifying the stability and/or translation efficiency of the RNA. For example, it is known that AU-rich elements in 3′UTR sequences can decrease the stability of mRNA. Therefore, 3′ UTRs can be selected or designed to increase the stability of the transcribed RNA based on properties of UTRs that are well known in the art.

In one embodiment, the 5′ UTR can contain the Kozak sequence of the endogenous nucleic acid. Alternatively, when a 5′ UTR that is not endogenous to the nucleic acid of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many mRNAs is known in the art. In other embodiments the 5′ UTR can be 5′UTR of an RNA virus whose RNA genome is stable in cells. In other embodiments various nucleotide analogues can be used in the 3′ or 5′ UTR to impede exonuclease degradation of the mRNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one preferred embodiment, the promoter is a T7 polymerase promoter, as described elsewhere herein. Other useful promoters include, but are not limited to, T3 and SP6 RNA polymerase promoters. Consensus nucleotide sequences for T7, T3 and SP6 promoters are known in the art.

In a preferred embodiment, the mRNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized mRNA which is not effective in eukaryotic transfection even if it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003).

The conventional method of integration of polyA/T stretches into a DNA template is molecular cloning. However polyA/T sequence integrated into plasmid DNA can cause plasmid instability, which is why plasmid DNA templates obtained from bacterial cells are often highly contaminated with deletions and other aberrations. This makes cloning procedures not only laborious and time consuming but often not reliable. That is why a method which allows construction of DNA templates with polyA/T 3′ stretch without cloning highly desirable.

The polyA/T segment of the transcriptional DNA template can be produced during PCR by using a reverse primer containing a polyT tail, such as 100 T tail (size can be 50-5000 Ts), or after PCR by any other method, including, but not limited to, DNA ligation or in vitro recombination. Poly(A) tails also provide stability to RNAs and reduce their degradation. Generally, the length of a poly(A) tail positively correlates with the stability of the transcribed RNA. In one embodiment, the poly(A) tail is between 100 and 5000 adenosines.

Poly(A) tails of RNAs can be further extended following in vitro transcription with the use of a poly(A) polymerase, such as E. coli polyA polymerase (E-PAP). In one embodiment, increasing the length of a poly(A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps on also provide stability to RNA molecules. In a preferred embodiment, RNAs produced by the methods disclosed herein include a 5′ cap. The 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

The RNAs produced by the methods disclosed herein can also contain an internal ribosome entry site (IRES) sequence. The IRES sequence may be any viral, chromosomal or artificially designed sequence which initiates cap-independent ribosome binding to mRNA and facilitates the initiation of translation. Any solutes suitable for cell electroporation, which can contain factors facilitating cellular permeability and viability such as sugars, peptides, lipids, proteins, antioxidants, and surfactants can be included.

RNA can be introduced into target cells using any of a number of different methods, for instance, commercially available methods which include, but are not limited to, electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany), cationic liposome mediated transfection using lipofection, polymer encapsulation, peptide mediated transfection, or biolistic particle delivery systems such as “gene guns” (see, for example, Nishikawa, et al. Hum Gene Ther., 12(8):861-70 (2001).

### Nucleic Acid Constructs Encoding a TFP

The present invention also provides nucleic acid molecules encoding one or more TFP constructs described herein. In one aspect, the nucleic acid molecule is provided as a messenger RNA transcript. In one aspect, the nucleic acid molecule is provided as a DNA construct.

The nucleic acid sequences coding for the desired molecules can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

The present invention also provides vectors in which a DNA of the present invention is inserted. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

In another embodiment, the vector comprising the nucleic acid encoding the desired TFP of the invention is an adenoviral vector (A5/35). In another embodiment, the expression of nucleic acids encoding TFPs can be accomplished using of transposons such as sleeping beauty, crisper, CAS9, and zinc finger nucleases (See, June et al. 2009 Nature Reviews Immunol. 9.10: 704-716, incorporated herein by reference).

The expression constructs of the present invention may also be used for nucleic acid immunization and gene therapy, using standard gene delivery protocols. Methods for gene delivery are known in the art (see, e.g., U.S. Pat. Nos. 5,399,346, 5,580,859, 5,589,466, incorporated by reference herein in their entireties). In another embodiment, the invention provides a gene therapy vector.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, e.g., in Sambrook et al., 2012, Molecular Cloning: A Laboratory Manual, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers (e.g., WO 01/96584; WO 01/29058; and U.S. Pat. No. 6,326,193).

A number of virally based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

Additional promoter elements, e.g., enhancers, regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

An example of a promoter that is capable of expressing a TFP transgene in a mammalian T cell is the EF1a promoter. The native EF1a promoter drives expression of the alpha subunit of the elongation factor-1 complex, which is responsible for the enzymatic delivery of aminoacyl tRNAs to the ribosome. The EF1a promoter has been extensively used in mammalian expression plasmids and has been shown to be effective in driving TFP expression from transgenes cloned into a lentiviral vector (see, e.g., Milone et al., Mol. Ther. 17(8): 1453-1464 (2009)). Another example of a promoter is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-1a promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline-regulated promoter.

In order to assess the expression of a TFP polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art (see, e.g., Sambrook et al., 2012, Molecular Cloning: A Laboratory Manual, volumes 1-4, Cold Spring Harbor Press, NY). One method for the introduction of a polynucleotide into a host cell is calcium phosphate transfection

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like (see, e.g., U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods of state-of-the-art targeted delivery of nucleic acids are available, such as delivery of polynucleotides with targeted nanoparticles or other suitable sub-micron sized delivery system.

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

The present invention further provides a vector comprising a TFP encoding nucleic acid molecule. In one aspect, a TFP vector can be directly transduced into a cell, e.g., a T cell. In one aspect, the vector is a cloning or expression vector, e.g., a vector including, but not limited to, one or more plasmids (e.g., expression plasmids, cloning vectors, minicircles, minivectors, double minute chromosomes), retroviral and lentiviral vector constructs. In one aspect, the vector is capable of expressing the TFP construct in mammalian T cells. In one aspect, the mammalian T cell is a human T cell.

### Sources of T Cells

Prior to expansion and genetic modification, a source of T cells is obtained from a subject. The term “subject” is intended to include living organisms in which an immune response can be elicited (e.g., mammals). Examples of subjects include humans, dogs, cats, mice, rats, and transgenic species thereof. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain aspects of the present invention, any number of T cell lines available in the art, may be used. In certain aspects of the present invention, T cells can be obtained from a unit of blood collected from a subject using any number of techniques known to the skilled artisan, such as Ficoll™ separation. In one preferred aspect, cells from the circulating blood of an individual are obtained by apheresis. The apheresis product typically contains lymphocytes, including T cells, monocytes, granulocytes, B cells, other nucleated white blood cells, red blood cells, and platelets. In one aspect, the cells collected by apheresis may be washed to remove the plasma fraction and to place the cells in an appropriate buffer or media for subsequent processing steps. In one aspect of the invention, the cells are washed with phosphate buffered saline (PBS). In an alternative aspect, the wash solution lacks calcium and may lack magnesium or may lack many if not all divalent cations. Initial activation steps in the absence of calcium can lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca-free, Mg-free PBS, PlasmaLyte A, or other saline solution with or without buffer. Alternatively, the undesirable components of the apheresis sample may be removed and the cells directly resuspended in culture media.

In one aspect, T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3+, CD28+, CD4+, CD8+, CD45RA+, and CD45RO+ T cells, can be further isolated by positive or negative selection techniques. For example, in one aspect, T cells are isolated by incubation with anti-CD³/anti-CD28 (e.g., 3×28)-conjugated beads, such as DYNABEADS™ M-450 CD3/CD28 T, for a time period sufficient for positive selection of the desired T cells. In one aspect, the time period is about 30 minutes. In a further aspect, the time period ranges from 30 minutes to 36 hours or longer and all integer values there between. In a further aspect, the time period is at least 1, 2, 3, 4, 5, or 6 hours. In yet another preferred aspect, the time period is 10 to 24 hours. In one aspect, the incubation time period is 24 hours. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used in the context of this invention. In certain aspects, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8. In certain aspects, it may be desirable to enrich for or positively select for regulatory T cells which typically express CD4+, CD25+, CD62Lhi, GITR+, and FoxP3+. Alternatively, in certain aspects, T regulatory cells are depleted by anti-C25 conjugated beads or other similar method of selection.

In one embodiment, a T cell population can be selected that expresses one or more of IFN-γ, TNF-alpha, IL-17A, IL-2, IL-3, IL-4, GM-CSF, IL-10, IL-13, granzyme B, and perforin, or other appropriate molecules, e.g., other cytokines. Methods for screening for cell expression can be determined, e.g., by the methods described in PCT Publication No.: WO 2013/126712.

For isolation of a desired population of cells by positive or negative selection, the concentration of cells and surface (e.g., particles such as beads) can be varied. In certain aspects, it may be desirable to significantly decrease the volume in which beads and cells are mixed together (e.g., increase the concentration of cells), to ensure maximum contact of cells and beads. For example, in one aspect, a concentration of 2 billion cells/mL is used. In one aspect, a concentration of 1 billion cells/mL is used. In a further aspect, greater than 100 million cells/mL is used. In a further aspect, a concentration of cells of 10, 15, 20, 25, 30, 35, 40, 45, or 50 million cells/mL is used. In yet one aspect, a concentration of cells from 75, 80, 85, 90, 95, or 100 million cells/mL is used. In further aspects, concentrations of 125 or 150 million cells/mL can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (e.g., leukemic blood, tumor tissue, etc.). Such populations of cells may have therapeutic value and would be desirable to obtain. For example, using high concentration of cells allows more efficient selection of CD8+ T cells that normally have weaker CD28 expression.

In a related aspect, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In one aspect, the concentration of cells used is 5×10⁶/mL. In other aspects, the concentration used can be from about 1×10⁵/mL to 1×10⁶/mL, and any integer value in between. In other aspects, the cells may be incubated on a rotator for varying lengths of time at varying speeds at either 2-10° C. or at room temperature.

T cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1 per minute and stored in the vapor phase of a liquid nitrogen storage tank. Other methods of controlled freezing may be used as well as uncontrolled freezing immediately at −20° C. or in liquid nitrogen. In certain aspects, cryopreserved cells are thawed and washed as described herein and allowed to rest for one hour at room temperature prior to activation using the methods of the present invention.

Also contemplated in the context of the invention is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in T cell therapy for any number of diseases or conditions that would benefit from T cell therapy, such as those described herein. In one aspect a blood sample or an apheresis is taken from a generally healthy subject. In certain aspects, a blood sample or an apheresis is taken from a generally healthy subject who is at risk of developing a disease, but who has not yet developed a disease, and the cells of interest are isolated and frozen for later use. In certain aspects, the T cells may be expanded, frozen, and used at a later time. In certain aspects, samples are collected from a patient shortly after diagnosis of a particular disease as described herein but prior to any treatments. In a further aspect, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as alemtuzumab, anti-CD3 antibodies, cytoxan, fludarabine, cyclosporin, FK506, rapamycin, mycophenolic acid, steroids, FR901228, and irradiation.

In a further aspect of the present invention, T cells are obtained from a patient directly following treatment that leaves the subject with functional T cells. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present invention to collect blood cells, including T cells, dendritic cells, or other cells of the hematopoietic lineage, during this recovery phase. Further, in certain aspects, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

### Activation and Expansion of T Cells

T cells may be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041, and 7,572,631.

Generally, the T cells of the invention may be expanded by contact with a surface having attached thereto an agent that stimulates a CD3/TCR complex associated signal and a ligand that stimulates a costimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody. Examples of an anti-CD28 antibody include 9.3, B-T3, XR-CD28 (Diaclone, Besancon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol. Meth. 227(1-2):53-63, 1999).

T cells that have been exposed to varied stimulation times may exhibit different characteristics. For example, typical blood or apheresed peripheral blood mononuclear cell products have a helper T cell population (TH, CD4+) that is greater than the cytotoxic or suppressor T cell population (TC, CD8+). Ex vivo expansion of T cells by stimulating CD3 and CD28 receptors produces a population of T cells that prior to about days 8-9 consists predominately of TH cells, while after about days 8-9, the population of T cells comprises an increasingly greater population of TC cells. Accordingly, depending on the purpose of treatment, infusing a subject with a T cell population comprising predominately of TH cells may be advantageous. Similarly, if an antigen-specific subset of TC cells has been isolated it may be beneficial to expand this subset to a greater degree.

Further, in addition to CD4 and CD8 markers, other phenotypic markers vary significantly, but in large part, reproducibly during the course of the cell expansion process. Thus, such reproducibility enables the ability to tailor an activated T cell product for specific purposes.

Once an anti-mesothelin TFP is constructed, various assays can be used to evaluate the activity of the molecule, such as but not limited to, the ability to expand T cells following antigen stimulation, sustain T cell expansion in the absence of re-stimulation, and anti-cancer activities in appropriate in vitro and animal models. Assays to evaluate the effects of an anti-mesothelin TFP are described in further detail below

Western blot analysis of TFP expression in primary T cells can be used to detect the presence of monomers and dimers (see, e.g., Milone et al., Molecular Therapy 17(8): 1453-1464 (2009)). Very briefly, T cells (1:1 mixture of CD4⁺ and CD8⁺ T cells) expressing the TFPs are expanded in vitro for more than 10 days followed by lysis and SDS-PAGE under reducing conditions. TFPs are detected by Western blotting using an antibody to a TCR chain. The same T cell subsets are used for SDS-PAGE analysis under non-reducing conditions to permit evaluation of covalent dimer formation.

In vitro expansion of TFP⁺ T cells following antigen stimulation can be measured by flow cytometry. For example, a mixture of CD4⁺ and CD8⁺ T cells are stimulated with alphaCD3/alphaCD28 and APCs followed by transduction with lentiviral vectors expressing GFP under the control of the promoters to be analyzed. Exemplary promoters include the CMV IE gene, EF-1alpha, ubiquitin C, or phosphoglycerokinase (PGK) promoters. GFP fluorescence is evaluated on day 6 of culture in the CD4+ and/or CD8+ T cell subsets by flow cytometry (see, e.g., Milone et al., Molecular Therapy 17(8): 1453-1464 (2009)). Alternatively, a mixture of CD4+ and CD8+ T cells are stimulated with alphaCD3/alphaCD28 coated magnetic beads on day 0, and transduced with TFP on day 1 using a bicistronic lentiviral vector expressing TFP along with eGFP using a 2A ribosomal skipping sequence. Cultures are re-stimulated with either mesothelin+K562 cells (K562-mesothelin), wild-type K562 cells (K562 wild type) or K562 cells expressing hCD32 and 4-1BBL in the presence of antiCD3 and anti-CD28 antibody (K562-BBL-3/28) following washing. Exogenous IL-2 is added to the cultures every other day at 100 IU/mL. GFP+ T cells are enumerated by flow cytometry using bead-based counting (see, e.g., Milone et al., Molecular Therapy 17(8): 1453-1464 (2009)).

Sustained TFP+ T cell expansion in the absence of re-stimulation can also be measured (see, e.g., Milone et al., Molecular Therapy 17(8): 1453-1464 (2009)). Briefly, mean T cell volume (fl) is measured on day 8 of culture using a Coulter Multisizer III particle counter following stimulation with alphaCD3/alphaCD28 coated magnetic beads on day 0, and transduction with the indicated TFP on day 1.

Animal models can also be used to measure a TFP-T activity. For example, xenograft model using human mesothelin-specific TFP+ T cells to treat a cancer in immunodeficient mice (see, e.g., Milone et al., Molecular Therapy 17(8): 1453-1464 (2009)). Very briefly, after establishment of cancer, mice are randomized as to treatment groups. Different numbers of engineered T cells are coinjected at a 1:1 ratio into NOD/SCID/γ−/− mice bearing cancer. The number of copies of each vector in spleen DNA from mice is evaluated at various times following T cell injection. Animals are assessed for cancer at weekly intervals. Peripheral blood mesothelin+ cancer cell counts are measured in mice that are injected with alphamesothelin-zeta TFP+ T cells or mock-transduced T cells. Survival curves for the groups are compared using the log-rank test. In addition, absolute peripheral blood CD4+ and CD8+ T cell counts 4 weeks following T cell injection in NOD/SCID/γ−/− mice can also be analyzed. Mice are injected with cancer cells and 3 weeks later are injected with T cells engineered to express TFP by a bicistronic lentiviral vector that encodes the TFP linked to eGFP. T cells are normalized to 45-50% input GFP+ T cells by mixing with mock-transduced cells prior to injection, and confirmed by flow cytometry. Animals are assessed for cancer at 1-week intervals. Survival curves for the TFP+ T cell groups are compared using the log-rank test.

Dose dependent TFP treatment response can be evaluated (see, e.g., Milone et al., Molecular Therapy 17(8): 1453-1464 (2009)). For example, peripheral blood is obtained 35-70 days after establishing cancer in mice injected on day 21 with TFP T cells, an equivalent number of mock-transduced T cells, or no T cells. Mice from each group are randomly bled for determination of peripheral blood mesothelin+ cancer cell counts and then killed on days 35 and 49. The remaining animals are evaluated on days 57 and 70.

Assessment of cell proliferation and cytokine production has been previously described, e.g., at Milone et al., Molecular Therapy 17(8): 1453-1464 (2009). Briefly, assessment of TFP-mediated proliferation is performed in microtiter plates by mixing washed T cells with cells expressing mesothelin or CD32 and CD137 (KT32-BBL) for a final T cell:cell expressing mesothelin ratio of 2:1. Cells expressing mesothelin cells are irradiated with gamma-radiation prior to use. Anti-CD3 (clone OKT3) and anti-CD28 (clone 9.3) monoclonal antibodies are added to cultures with KT32-BBL cells to serve as a positive control for stimulating T cell proliferation since these signals support long-term CD8+ T cell expansion ex vivo. T cells are enumerated in cultures using CountBright™ fluorescent beads (Invitrogen) and flow cytometry as described by the manufacturer. TFP+ T cells are identified by GFP expression using T cells that are engineered with eGFP-2A linked TFP-expressing lentiviral vectors. For TFP+ T cells not expressing GFP, the TFP+ T cells are detected with biotinylated recombinant mesothelin protein and a secondary avidin-PE conjugate. CD4+ and CD8+ expression on T cells are also simultaneously detected with specific monoclonal antibodies (BD Biosciences). Cytokine measurements are performed on supernatants collected 24 hours following re-stimulation using the human TH1/TH2 cytokine cytometric bead array kit (BD Biosciences) according the manufacturer's instructions. Fluorescence is assessed using a FACScalibur flow cytometer, and data is analyzed according to the manufacturer's instructions.

Cytotoxicity can be assessed by a standard ⁵¹Cr-release assay (see, e.g., Milone et al., Molecular Therapy 17(8): 1453-1464 (2009)). Briefly, target cells are loaded with ⁵¹Cr (as NaCrO₄, New England Nuclear) at 37° C. for 2 hours with frequent agitation, washed twice in complete RPMI medium and plated into microtiter plates. Effector T cells are mixed with target cells in the wells in complete RPMI at varying ratios of effector cell:target cell (E:T). Additional wells containing media only (spontaneous release, SR) or a 1% solution of triton-X 100 detergent (total release, TR) are also prepared. After 4 hours of incubation at 37° C., supernatant from each well is harvested. Released ⁵¹Cr is then measured using a gamma particle counter (Packard Instrument Co., Waltham, Mass.). Each condition is performed in at least triplicate, and the percentage of lysis is calculated using the formula: % Lysis=(ER−SR)/(TR−SR), where ER represents the average ⁵¹Cr released for each experimental condition.

Imaging technologies can be used to evaluate specific trafficking and proliferation of TFPs in tumor-bearing animal models. Such assays have been described, e.g., in Barrett et al., Human Gene Therapy 22:1575-1586 (2011). Briefly, NOD/SCID/γc−/− (NSG) mice are injected IV with cancer cells followed 7 days later with T cells 4 hour after electroporation with the TFP constructs. The T cells are stably transfected with a lentiviral construct to express firefly luciferase, and mice are imaged for bioluminescence. Alternatively, therapeutic efficacy and specificity of a single injection of TFP+ T cells in a cancer xenograft model can be measured as follows: NSG mice are injected with cancer cells transduced to stably express firefly luciferase, followed by a single tail-vein injection of T cells electroporated with mesothelin TFP 7 days later. Animals are imaged at various time points post injection. For example, photon-density heat maps of firefly luciferase positive cancer in representative mice at day 5 (2 days before treatment) and day 8 (24 hours post TFP+ PBLs) can be generated.

Other assays, including those described in the Example section herein as well as those that are known in the art can also be used to evaluate the anti-mesothelin TFP constructs of the invention.

Therapeutic Applications

Mesothelin Associated Diseases and/or Disorders

In one aspect, the invention provides methods for treating a disease associated with mesothelin expression. In one aspect, the invention provides methods for treating a disease wherein part of the tumor is negative for mesothelin and part of the tumor is positive for mesothelin. For example, the TFP of the invention is useful for treating subjects that have undergone treatment for a disease associated with elevated expression of mesothelin, wherein the subject that has undergone treatment for elevated levels of mesothelin exhibits a disease associated with elevated levels of mesothelin.

In one aspect, the invention pertains to a vector comprising anti-mesothelin TFP operably linked to promoter for expression in mammalian T cells. In one aspect, the invention provides a recombinant T cell expressing the mesothelin TFP for use in treating mesothelin-expressing tumors, wherein the recombinant T cell expressing the mesothelin TFP is termed a mesothelin TFP-T. In one aspect, the mesothelin TFP-T of the invention is capable of contacting a tumor cell with at least one mesothelin TFP of the invention expressed on its surface such that the TFP-T targets the tumor cell and growth of the tumor is inhibited.

In one aspect, the invention pertains to a method of inhibiting growth of a mesothelin-expressing tumor cell, comprising contacting the tumor cell with a mesothelin TFP T cell of the present invention such that the TFP-T is activated in response to the antigen and targets the cancer cell, wherein the growth of the tumor is inhibited.

In one aspect, the invention pertains to a method of treating cancer in a subject. The method comprises administering to the subject a mesothelin TFP T cell of the present invention such that the cancer is treated in the subject. An example of a cancer that is treatable by the mesothelin TFP T cell of the invention is a cancer associated with expression of mesothelin. In one aspect, the cancer is a mesothelioma. In one aspect, the cancer is a pancreatic cancer. In one aspect, the cancer is an ovarian cancer. In one aspect, the cancer is a stomach cancer. In one aspect, the cancer is a lung cancer. In one aspect, the cancer is an endometrial cancer. In some embodiments, mesothelin TFP therapy can be used in combination with one or more additional therapies.

The invention includes a type of cellular therapy where T cells are genetically modified to express a TFP and the TFP-expressing T cell is infused to a recipient in need thereof. The infused cell is able to kill tumor cells in the recipient. Unlike antibody therapies, TFP-expressing T cells are able to replicate in vivo, resulting in long-term persistence that can lead to sustained tumor control. In various aspects, the T cells administered to the patient, or their progeny, persist in the patient for at least one month, two month, three months, four months, five months, six months, seven months, eight months, nine months, ten months, eleven months, twelve months, thirteen months, fourteen month, fifteen months, sixteen months, seventeen months, eighteen months, nineteen months, twenty months, twenty-one months, twenty-two months, twenty-three months, two years, three years, four years, or five years after administration of the T cell to the patient.

The invention also includes a type of cellular therapy where T cells are modified, e.g., by in vitro transcribed RNA, to transiently express a TFP and the TFP-expressing T cell is infused to a recipient in need thereof. The infused cell is able to kill tumor cells in the recipient. Thus, in various aspects, the T cells administered to the patient, is present for less than one month, e.g., three weeks, two weeks, or one week, after administration of the T cell to the patient.

Without wishing to be bound by any particular theory, the anti-tumor immunity response elicited by the TFP-expressing T cells may be an active or a passive immune response, or alternatively may be due to a direct vs indirect immune response. In one aspect, the TFP transduced T cells exhibit specific proinflammatory cytokine secretion and potent cytolytic activity in response to human cancer cells expressing the mesothelin antigen, resist soluble mesothelin inhibition, mediate bystander killing and/or mediate regression of an established human tumor. For example, antigen-less tumor cells within a heterogeneous field of mesothelin-expressing tumor may be susceptible to indirect destruction by mesothelin-redirected T cells that has previously reacted against adjacent antigen-positive cancer cells.

In one aspect, the human TFP-modified T cells of the invention may be a type of vaccine for ex vivo immunization and/or in vivo therapy in a mammal. In one aspect, the mammal is a human.

With respect to ex vivo immunization, at least one of the following occurs in vitro prior to administering the cell into a mammal: i) expansion of the cells, ii) introducing a nucleic acid encoding a TFP to the cells or iii) cryopreservation of the cells.

Ex vivo procedures are well known in the art and are discussed more fully below. Briefly, cells are isolated from a mammal (e.g., a human) and genetically modified (i.e., transduced or transfected in vitro) with a vector expressing a TFP disclosed herein. The TFP-modified cell can be administered to a mammalian recipient to provide a therapeutic benefit. The mammalian recipient may be a human and the TFP-modified cell can be autologous with respect to the recipient. Alternatively, the cells can be allogeneic, syngeneic or xenogeneic with respect to the recipient.

The procedure for ex vivo expansion of hematopoietic stem and progenitor cells is described in U.S. Pat. No. 5,199,942, incorporated herein by reference, can be applied to the cells of the present invention. Other suitable methods are known in the art, therefore the present invention is not limited to any particular method of ex vivo expansion of the cells. Briefly, ex vivo culture and expansion of T cells comprises: (1) collecting CD34+ hematopoietic stem and progenitor cells from a mammal from peripheral blood harvest or bone marrow explants; and (2) expanding such cells ex vivo. In addition to the cellular growth factors described in U.S. Pat. No. 5,199,942, other factors such as flt3-L, IL-1, IL-3 and c-kit ligand, can be used for culturing and expansion of the cells.

In addition to using a cell-based vaccine in terms of ex vivo immunization, the present invention also provides compositions and methods for in vivo immunization to elicit an immune response directed against an antigen in a patient.

Generally, the cells activated and expanded as described herein may be utilized in the treatment and prevention of diseases that arise in individuals who are immunocompromised. In particular, the TFP-modified T cells of the invention are used in the treatment of diseases, disorders and conditions associated with expression of mesothelin. In certain aspects, the cells of the invention are used in the treatment of patients at risk for developing diseases, disorders and conditions associated with expression of mesothelin. Thus, the present invention provides methods for the treatment or prevention of diseases, disorders and conditions associated with expression of mesothelin comprising administering to a subject in need thereof, a therapeutically effective amount of the TFP-modified T cells of the invention.

In one aspect the TFP-T cells of the inventions may be used to treat a proliferative disease such as a cancer or malignancy or a precancerous condition. In one aspect, the cancer is a mesothelioma. In one aspect, the cancer is a pancreatic cancer. In one aspect, the cancer is an ovarian cancer. In one aspect, the cancer is a stomach cancer. In one aspect, the cancer is a lung cancer. In one aspect, the cancer is a endometrial cancer. Further a disease associated with mesothelin expression includes, but is not limited to, e.g., atypical and/or non-classical cancers, malignancies, precancerous conditions or proliferative diseases expressing mesothelin. Non-cancer related indications associated with expression of mesothelin include, but are not limited to, e.g., autoimmune disease, (e.g., lupus), inflammatory disorders (allergy and asthma) and transplantation.

The TFP-modified T cells of the present invention may be administered either alone, or as a pharmaceutical composition in combination with diluents and/or with other components such as IL-2 or other cytokines or cell populations.

The present invention also provides methods for inhibiting the proliferation or reducing a mesothelin-expressing cell population, the methods comprising contacting a population of cells comprising a mesothelin-expressing cell with an anti-mesothelin TFP-T cell of the invention that binds to the mesothelin-expressing cell. In a specific aspect, the present invention provides methods for inhibiting the proliferation or reducing the population of cancer cells expressing mesothelin, the methods comprising contacting the mesothelin-expressing cancer cell population with an anti-mesothelin TFP-T cell of the invention that binds to the mesothelin-expressing cell. In one aspect, the present invention provides methods for inhibiting the proliferation or reducing the population of cancer cells expressing mesothelin, the methods comprising contacting the mesothelin-expressing cancer cell population with an anti-mesothelin TFP-T cell of the invention that binds to the mesothelin-expressing cell. In certain aspects, the anti-mesothelin TFP-T cell of the invention reduces the quantity, number, amount or percentage of cells and/or cancer cells by at least 25%, at least 30%, at least 40%, at least 50%, at least 65%, at least 75%, at least 85%, at least 95%, or at least 99% in a subject with or animal model a cancer associated with mesothelin-expressing cells relative to a negative control. In one aspect, the subject is a human.

The present invention also provides methods for preventing, treating and/or managing a disease associated with mesothelin-expressing cells (e.g., a cancer expressing mesothelin), the methods comprising administering to a subject in need an anti-mesothelin TFP-T cell of the invention that binds to the mesothelin-expressing cell. In one aspect, the subject is a human. Non-limiting examples of disorders associated with mesothelin-expressing cells include autoimmune disorders (such as lupus), inflammatory disorders (such as allergies and asthma) and cancers (such as pancreatic cancer, ovarian cancer, stomach cancer, lung cancer, or endometrial cancer. or atypical cancers expressing mesothelin).

The present invention also provides methods for preventing, treating and/or managing a disease associated with mesothelin-expressing cells, the methods comprising administering to a subject in need an anti-mesothelin TFP-T cell of the invention that binds to the mesothelin-expressing cell. In one aspect, the subject is a human.

The present invention provides methods for preventing relapse of cancer associated with mesothelin-expressing cells, the methods comprising administering to a subject in need thereof an anti-mesothelin TFP-T cell of the invention that binds to the mesothelin-expressing cell. In one aspect, the methods comprise administering to the subject in need thereof an effective amount of an anti-mesothelin TFP-T cell described herein that binds to the mesothelin-bmca expressing cell in combination with an effective amount of another therapy.

### Combination Therapies

A TFP-expressing cell described herein may be used in combination with other known agents and therapies. Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In some embodiments, the “at least one additional therapeutic agent” includes a TFP-expressing cell. Also provided are T cells that express multiple TFPs, which bind to the same or different target antigens, or same or different epitopes on the same target antigen. Also provided are populations of T cells in which a first subset of T cells express a first TFP and a second subset of T cells express a second TFP.

A TFP-expressing cell described herein and the at least one additional therapeutic agent can be administered simultaneously, in the same or in separate compositions, or sequentially. For sequential administration, the TFP-expressing cell described herein can be administered first, and the additional agent can be administered second, or the order of administration can be reversed.

In further aspects, a TFP-expressing cell described herein may be used in a treatment regimen in combination with surgery, chemotherapy, radiation, immunosuppressive agents, such as cyclosporin, azathioprine, methotrexate, mycophenolate, and FK506, antibodies, or other immunoablative agents such as alemtuzumab, anti-CD3 antibodies or other antibody therapies, cytoxin, fludarabine, cyclosporin, FK506, rapamycin, mycophenolic acid, steroids, FR901228, cytokines, and irradiation. A TFP-expressing cell described herein may also be used in combination with a peptide vaccine, such as that described in Izumoto et al. 2008 J Neurosurg 108:963-971. In a further aspect, a TFP-expressing cell described herein may also be used in combination with a promoter of myeloid cell differentiation (e.g., all-trans retinoic acid), an inhibitor of myeloid-derived suppressor cell (MDSC) expansion (e.g., inhibitors of c-kit receptor or a VEGF inhibitor), an inhibition of MDSC function (e.g., COX2 inhibitors or phosphodiesterase-5 inhibitors), or therapeutic elimination of MDSCs (e.g., with a chemotherapeutic regimen such as treatment with doxorubicin and cyclophosphamide). Other therapeutic agents that may prevent the expansion of MDSCs include amino-biphosphonate, biphosphanate, sildenafil and tadalafil, nitroaspirin, vitamin D3, and gemcitabine. (See, e.g., Gabrilovich and Nagaraj, Nat. Rev. Immunol, (2009) v9(3): 162-174).

In one embodiment, the subject can be administered an agent which reduces or ameliorates a side effect associated with the administration of a TFP-expressing cell. Side effects associated with the administration of a TFP-expressing cell include, but are not limited to cytokine release syndrome (CRS), and hemophagocytic lymphohistiocytosis (HLH), also termed Macrophage Activation Syndrome (MAS). Symptoms of CRS include high fevers, nausea, transient hypotension, hypoxia, and the like. Accordingly, the methods described herein can comprise administering a TFP-expressing cell described herein to a subject and further administering an agent to manage elevated levels of a soluble factor resulting from treatment with a TFP-expressing cell. In one embodiment, the soluble factor elevated in the subject is one or more of IFN-γ, TNFα, IL-2, IL-6 and IL8. Therefore, an agent administered to treat this side effect can be an agent that neutralizes one or more of these soluble factors. Such agents include, but are not limited to a steroid, an inhibitor of TNFα, and an inhibitor of IL-6. An example of a TNFα inhibitor is entanercept. An example of an IL-6 inhibitor is tocilizumab (toc).

In one embodiment, the subject can be administered an agent which enhances the activity of a TFP-expressing cell. For example, in one embodiment, the agent can be an agent which inhibits an inhibitory molecule. Inhibitory molecules, e.g., Programmed Death 1 (PD1), can, in some embodiments, decrease the ability of a TFP-expressing cell to mount an immune effector response. Examples of inhibitory molecules include PD1, PD-L1, CTLA4, TIM3, LAG3, VISTA, BTLA, TIGIT, LAIR1, CD160, 2B4 and TGFR beta. Inhibition of an inhibitory molecule, e.g., by inhibition at the DNA, RNA or protein level, can optimize a TFP-expressing cell performance. In embodiments, an inhibitory nucleic acid, e.g., an inhibitory nucleic acid, e.g., a dsRNA, e.g., an siRNA or shRNA, can be used to inhibit expression of an inhibitory molecule in the TFP-expressing cell. In an embodiment the inhibitor is a shRNA. In an embodiment, the inhibitory molecule is inhibited within a TFP-expressing cell. In these embodiments, a dsRNA molecule that inhibits expression of the inhibitory molecule is linked to the nucleic acid that encodes a component, e.g., all of the components, of the TFP. In one embodiment, the inhibitor of an inhibitory signal can be, e.g., an antibody or antibody fragment that binds to an inhibitory molecule. For example, the agent can be an antibody or antibody fragment that binds to PD1, PD-L1, PD-L2 or CTLA4 (e.g., ipilimumab (also referred to as MDX-010 and MDX-101, and marketed as Yervoy™; Bristol-Myers Squibb; tremelimumab (IgG2 monoclonal antibody available from Pfizer, formerly known as ticilimumab, CP-675,206)). In an embodiment, the agent is an antibody or antibody fragment that binds to TIM3. In an embodiment, the agent is an antibody or antibody fragment that binds to LAG3.

In some embodiments, the T cells may be altered (e.g., by gene transfer) in vivo via a lentivirus, e.g., a lentivirus specifically targeting a CD4+ or CD8+ T cell. (See, e.g., Zhou et al., J. Immunol. (2015) 195:2493-2501).

In some embodiments, the agent which enhances the activity of a TFP-expressing cell can be, e.g., a fusion protein comprising a first domain and a second domain, wherein the first domain is an inhibitory molecule, or fragment thereof, and the second domain is a polypeptide that is associated with a positive signal, e.g., a polypeptide comprising an intracellular signaling domain as described herein. In some embodiments, the polypeptide that is associated with a positive signal can include a costimulatory domain of CD28, CD27, ICOS, e.g., an intracellular signaling domain of CD28, CD27 and/or ICOS, and/or a primary signaling domain, e.g., of CD3 zeta, e.g., described herein. In one embodiment, the fusion protein is expressed by the same cell that expressed the TFP. In another embodiment, the fusion protein is expressed by a cell, e.g., a T cell that does not express an anti-mesothelin TFP.

### Pharmaceutical Compositions

Pharmaceutical compositions of the present invention may comprise a TFP-expressing cell, e.g., a plurality of TFP-expressing cells, as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are in one aspect formulated for intravenous administration.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

In one embodiment, the pharmaceutical composition is substantially free of, e.g., there are no detectable levels of a contaminant, e.g., selected from the group consisting of endotoxin, mycoplasma, replication competent lentivirus (RCL), p24, VSV-G nucleic acid, HIV gag, residual anti-CD3/anti-CD28 coated beads, mouse antibodies, pooled human serum, bovine serum albumin, bovine serum, culture media components, vector packaging cell or plasmid components, a bacterium and a fungus. In one embodiment, the bacterium is at least one selected from the group consisting of Alcaligenes faecalis, Candida albicans, Escherichia coli, Haemophilus influenza, Neisseria meningitides, Pseudomonas aeruginosa, Staphylococcus aureus, Streptococcus pneumonia, and Streptococcus pyogenes group A.

When “an immunologically effective amount,” “an anti-tumor effective amount,” “a tumor-inhibiting effective amount,” or “therapeutic amount” is indicated, the precise amount of the compositions of the present invention to be administered can be determined by a physician with consideration of individual differences in age, weight, tumor size, extent of infection or metastasis, and condition of the patient (subject). It can generally be stated that a pharmaceutical composition comprising the T cells described herein may be administered at a dosage of 10⁴ to 10⁹ cells/kg body weight, in some instances 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988).

In certain aspects, it may be desired to administer activated T cells to a subject and then subsequently redraw blood (or have an apheresis performed), activate T cells therefrom according to the present invention, and reinfuse the patient with these activated and expanded T cells. This process can be carried out multiple times every few weeks. In certain aspects, T cells can be activated from blood draws of from 10 cc to 400 cc. In certain aspects, T cells are activated from blood draws of 20 cc, 30 cc, 40 cc, 50 cc, 60 cc, 70 cc, 80 cc, 90 cc, or 100 cc.

The administration of the subject compositions may be carried out in any convenient manner, including by aerosol inhalation, injection, ingestion, transfusion, implantation or transplantation. The compositions described herein may be administered to a patient trans arterially, subcutaneously, intradermally, intratumorally, intranodally, intramedullary, intramuscularly, by intravenous (i.v.) injection, or intraperitoneally. In one aspect, the T cell compositions of the present invention are administered to a patient by intradermal or subcutaneous injection. In one aspect, the T cell compositions of the present invention are administered by i.v. injection. The compositions of T cells may be injected directly into a tumor, lymph node, or site of infection.

In a particular exemplary aspect, subjects may undergo leukapheresis, wherein leukocytes are collected, enriched, or depleted ex vivo to select and/or isolate the cells of interest, e.g., T cells. These T cell isolates may be expanded by methods known in the art and treated such that one or more TFP constructs of the invention may be introduced, thereby creating a TFP-expressing T cell of the invention. Subjects in need thereof may subsequently undergo standard treatment with high dose chemotherapy followed by peripheral blood stem cell transplantation. In certain aspects, following or concurrent with the transplant, subjects receive an infusion of the expanded TFP T cells of the present invention. In an additional aspect, expanded cells are administered before or following surgery.

The dosage of the above treatments to be administered to a patient will vary with the precise nature of the condition being treated and the recipient of the treatment. The scaling of dosages for human administration can be performed according to art-accepted practices. The dose for alemtuzumab, for example, will generally be in the range 1 to about 100 mg for an adult patient, usually administered daily for a period between 1 and 30 days. The preferred daily dose is 1 to 10 mg per day although in some instances larger doses of up to 40 mg per day may be used (described in U.S. Pat. No. 6,120,766).

In one embodiment, the TFP is introduced into T cells, e.g., using in vitro transcription, and the subject (e.g., human) receives an initial administration of TFP T cells of the invention, and one or more subsequent administrations of the TFP T cells of the invention, wherein the one or more subsequent administrations are administered less than 15 days, e.g., 14, 13, 12, 11, 10, 9, 8, 7, 6, 5, 4, 3, or 2 days after the previous administration. In one embodiment, more than one administration of the TFP T cells of the invention are administered to the subject (e.g., human) per week, e.g., 2, 3, or 4 administrations of the TFP T cells of the invention are administered per week. In one embodiment, the subject (e.g., human subject) receives more than one administration of the TFP T cells per week (e.g., 2, 3 or 4 administrations per week) (also referred to herein as a cycle), followed by a week of no TFP T cells administrations, and then one or more additional administration of the TFP T cells (e.g., more than one administration of the TFP T cells per week) is administered to the subject. In another embodiment, the subject (e.g., human subject) receives more than one cycle of TFP T cells, and the time between each cycle is less than 10, 9, 8, 7, 6, 5, 4, or 3 days. In one embodiment, the TFP T cells are administered every other day for 3 administrations per week. In one embodiment, the TFP T cells of the invention are administered for at least two, three, four, five, six, seven, eight or more weeks.

In one aspect, mesothelin TFP T cells are generated using lentiviral viral vectors, such as lentivirus. TFP-T cells generated that way will have stable TFP expression.

In one aspect, TFP T cells transiently express TFP vectors for 4, 5, 6, 7, 8, 9, 10, 11, 12, 13, 14, 15 days after transduction. Transient expression of TFPs can be effected by RNA TFP vector delivery. In one aspect, the TFP RNA is transduced into the T cell by electroporation.

A potential issue that can arise in patients being treated using transiently expressing TFP T cells (particularly with murine scFv bearing TFP T cells) is anaphylaxis after multiple treatments.

Without being bound by this theory, it is believed that such an anaphylactic response might be caused by a patient developing humoral anti-TFP response, i.e., anti-TFP antibodies having an anti-IgE isotype. It is thought that a patient's antibody producing cells undergo a class switch from IgG isotype (that does not cause anaphylaxis) to IgE isotype when there is a ten to fourteen-day break in exposure to antigen.

If a patient is at high risk of generating an anti-TFP antibody response during the course of transient TFP therapy (such as those generated by RNA transductions), TFP T cell infusion breaks should not last more than ten to fourteen days.

### Cytokine Release

Cytokine release syndrome is a form of systemic inflammatory response syndrome that arises as a complication of some diseases or infections, and is also an adverse effect of some monoclonal antibody drugs, as well as adoptive T cell therapies. TFP T cells can exhibit better killing activity than CAR-T cells. TFP T cells administered to a subject can exhibit better killing activity than CAR-T cells administered to a subject. This can be one of the advantages of TFP T cells over CAR-T cells. TFP T cells can exhibit less cytokine release CAR-T cells. A subject administered TFP T cells can exhibit less cytokine release than a subject administered CAR-T cells. This can be one of the advantages of TFP T cell therapies over CAR-T cell therapies. TFP T cells can exhibit similar or better killing activity than CAR-T cells and the TFP T cells can exhibit less cytokine release than the CAR-T cells. TFP T cells administered to a subject can exhibit similar or better killing activity than CAR-T cells administered to a subject and the subject can exhibit less cytokine release than a subject administered CAR-T cells. This can be one of the advantages of TFP T cell therapies over CAR-T cell therapies.

In some cases, the cytokine release of a treatment with TFP T cells is less than the cytokine release of a treatment with CAR-T cells. In some embodiments, the cytokine release of a treatment with TFP T cells is at least 10%, at least 20%, at least 30%, at least 40%, at least 50%, at least 60%, at least 70%, at least 80%, or at least 90% less than the cytokine release of a treatment with CAR-T cells. Various cytokines can be released less in the T cell treatment with TFP T cells than CAR-T cells. In some embodiments, the cytokine is IL-2, IFN-γ, IL-4, TNF-α, IL-6, IL-13, IL-5, IL-10, sCD137, GM-CSF, MIP-1α, MIP-10, or a combination thereof. In some cases, the treatment with TFP T cells release less perforin, granzyme A, granzyme B, or a combination thereof, than the treatment with CAR-T cells. In some embodiments, the perforin, granzyme A, or granzyme B released in a treatment with TFP T cells is at least 10%, at least 20%, at least 30%, at least 40%, at least 50%, or at least 60% less than a treatment with CAR-T cells.

In some embodiments, for a given cytokine, at least 10% less amount of the given cytokine is released following treatment compared to an amount of the given cytokine of a mammal treated with a CAR-T cell comprising the same human or humanized antibody domain. In some embodiments, the given cytokine comprises one or more cytokines selected from the group consisting of IL-2, IFN-γ, IL-4, TNF-α, IL-6, IL-13, IL-5, IL-10, sCD137, GM-CSF, MIP-1α, MIP-1β, and any combinations thereof.

The TFP T cells may exhibit similar or better activity in killing tumor cells than CAR-T cells. In some embodiments, a tumor growth in the mammal is inhibited such that a size of the tumor is at most 10%, at most 20%, at most 30%, at most 40%, at most 50%, or at most 60% of a size of a tumor in a mammal treated with T cells that do not express the TFP after at least 8 days of treatment, wherein the mammal treated with T cells expressing TFP and the mammal treated with T cells that do not express the TFP have the same tumor size before the treatment. In some embodiments, the tumor growth in the mammal is completely inhibited. In some embodiments, the tumor growth in the mammal is completely inhibited for at least 20 days, at least 30 days, at least 40 days, at least 50 days, at least 60 days, at least 70 days, at least 80 days, at least 90 days, at least 100 days, or more. In some embodiments, the population of T cells transduced with TFP kill similar amount of tumor cells compared to the CAR-T cells comprising the same human or humanized antibody domain.

The TFP T cells can exhibit different gene expression profile than cells that do not express TFP. In some cases, the TFP T cells may exhibit similar gene expression profiles than CAR-T cells. In some other cases, the TFP T cells may exhibit different gene expression profiles than CAR-T cells. In some embodiments, the population of T cells transduced with TFP have a different gene expression profile than the CAR-T cells comprising the same human or humanized antibody domain. In some embodiments, an expression level of a gene is different in the T cells transduced with the TFP than an expression level of the gene in the CAR-T cells comprising the same human or humanized antibody domain. In some embodiments, the gene has a function in antigen presentation, TCR signaling, homeostasis, metabolism, chemokine signaling, cytokine signaling, toll like receptor signaling, MMP and adhesion molecule signaling, or TNFR related signaling.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein. Without further description, it is believed that one of ordinary skill in the art can, using the preceding description and the following illustrative examples, make and utilize the compounds of the present invention and practice the claimed methods. The following working examples specifically point out various aspects of the present invention, and are not to be construed as limiting in any way the remainder of the disclosure. As used herein, in some cases, “TRuC” is used to refer to “TFP”.

### Example 1: TFP Constructs

Anti-mesothelin TFP constructs are engineered by cloning an anti-mesothelin scFv DNA fragment linked to a CD3 or TCR DNA fragment by either a DNA sequence encoding a short linker (SL): AAAGGGGSGGGGSGGGGSLE (SEQ ID NO:2) or a long linker (LL): AAAIEVMYPPPYLGGGGSGGGGSGGGGSLE (SEQ ID NO:3) into p510 vector ((System Biosciences (SBI)) at XbaI and EcoR1 sites.

The anti-mesothelin TFP constructs generated are p510_antimesothelin_LL_TCRα (anti-mesothelin scFv-long linker-human full length T cell receptor α chain), p510_antimesothelin_LL_TCR αC (anti-mesothelin scFv-long linker-human T cell receptor α constant domain chain), p510_antimesothelin_LL_TCRβ (anti-mesothelin scFv-long linker-human full length T cell receptor β chain), p510_antimesothelin_LL_TCRβC (anti-mesothelin scFv-long linker-human T cell receptor β constant domain chain), p510_antimesothelin_LL_CD3γ (anti-mesothelin scFv-long linker-human CD3γ chain), p510_antimesothelin_LL_CD3δ (anti-mesothelin scFv-long linker-human CD3δ chain), p510_antimesothelin_LL_CD3ε (anti-mesothelin scFv-long linker-human CD3ε chain), p510_antimesothelin_SL_TCRβ (anti-mesothelin scFv-short linker-human full length T cell receptor β chain), p510_antimesothelin_SL_CD3γ (anti-mesothelin scFv-short linker-human CD3γ chain), p510_antimesothelin_SL_CD3δ (anti-mesothelin scFv-short linker-human CD3δ chain), p510_antimesothelin_SL_CD3ε (anti-mesothelin scFv-short linker-human CD3ε chain).

The anti-mesothelin CAR construct, p510_antimesothelin_28ζ is generated by cloning synthesized DNA encoding anti-mesothelin, partial CD28 extracellular domain, CD28 transmembrane domain, CD28 intracellular domain and CD3 zeta into p510 vector at XbaI and EcoR1 sites.

### Example 2: Antibody Sequences

### Generation of Antibody Sequences

Generation of scFvs

Human or humanized anti-mesothelin IgGs are used to generate scFv sequences for TFP constructs. DNA sequences coding for human or humanized V_{L} and V_{H} domains are obtained, and the codons for the constructs are, optionally, optimized for expression in cells from Homo sapiens. The order in which the V_{L} and V_{H} domains appear in the scFv is varied (i.e., V_{L}-V_{H}, or V_{H}-V_{L} orientation), and three copies of the “G4S” or “G₄S” subunit (G₄S)₃ connect the variable domains to create the scFv domain. Anti-mesothelin scFv plasmid constructs can have optional Flag, His or other affinity tags, and are electroporated into HEK293 or other suitable human or mammalian cell lines and purified. Validation assays include binding analysis by FACS, kinetic analysis using Proteon, and staining of mesothelin-expressing cells.

Exemplary anti-mesothelin V_{L} and V_{H} domains, CDRs, and the nucleotide sequences encoding them, can be those described in U.S. Pat. Nos. 9,272,002; 8,206,710; 9,023,351; 7,081,518; 8,911,732; 9,115,197 and 9,416,190; and U.S. Patent Publication No. 20090047211. Other exemplary anti-mesothelin V_{L} and V_{H} domains, CDRs, and the nucleotide sequences encoding them, respectively, can be those of the following monoclonal antibodies: rat anti-mesothelin antibody 420411, rat anti-mesothelin antibody 420404, mouse anti-mesothelin antibody MN-1, mouse anti-mesothelin antibody MB-G10, mouse anti-mesothelin antibody ABIN233753, rabbit anti-mesothelin antibody FQS3796(3), rabbit anti-mesothelin antibody TQ85, mouse anti-mesothelin antibody TA307799, rat anti-mesothelin antibody 295D, rat anti-mesothelin antibody B35, mouse anti-mesothelin antibody 5G157, mouse anti-mesothelin antibody 129588, rabbit anti-mesothelin antibody 11C187, mouse anti-mesothelin antibody 5B2, rabbit anti-mesothelin antibody SP74, rabbit anti-mesothelin antibody D4X7M, mouse anti-mesothelin antibody C-2, mouse anti-mesothelin antibody C-3, mouse anti-mesothelin antibody G-1, mouse anti-mesothelin antibody G-4, mouse anti-mesothelin antibody K1, mouse anti-mesothelin antibody B-3, mouse anti-mesothelin antibody 200-301-A87, mouse anti-mesothelin antibody 200-301-A88, rabbit anti-mesothelin antibody EPR2685(2), rabbit anti-mesothelin antibody EPR4509, or rabbit anti-mesothelin antibody PPI-2e(IHC).

The human mesothelin polypeptide canonical sequence is UniProt Accession No. Q13421 (or Q13421-1). Provided are antibody polypeptides that are capable of specifically binding to the human mesothelin polypeptide, and fragments or domains thereof. Anti-mesothelin antibodies can be generated using diverse technologies (see, e.g., (Nicholson et al, 1997). Where murine anti-mesothelin antibodies are used as a starting material, humanization of murine anti-mesothelin antibodies is desired for the clinical setting, where the mouse-specific residues may induce a human-anti-mouse antigen (HAMA) response in subjects who receive T cell receptor (TCR) fusion protein (TFP) treatment, i.e., treatment with T cells transduced with the TFP.mesothelin construct. Humanization is accomplished by grafting CDR regions from murine anti-mesothelin antibody onto appropriate human germline acceptor frameworks, optionally including other modifications to CDR and/or framework regions. As provided herein, antibody and antibody fragment residue numbering follows Kabat (Kabat E. A. et al, 1991; Chothia et al, 1987).

In some embodiments, single-domain (V_{HH}) binders are used such as those set forth in SEQ ID NOS 53-55 (SD1, SD4, and SD6, respectively).

### Source of TCR Subunits

Subunits of the human T Cell Receptor (TCR) complex all contain an extracellular domain, a transmembrane domain, and an intracellular domain. A human TCR complex contains the CD3-epsilon polypeptide, the CD3-gamma polypeptide, the CD3-delta polypeptide, the CD3-zeta polypeptide, the TCR alpha chain polypeptide and the TCR beta chain polypeptide. The human CD3-epsilon polypeptide canonical sequence is Uniprot Accession No. P07766. The human CD3-gamma polypeptide canonical sequence is Uniprot Accession No. P09693. The human CD3-delta polypeptide canonical sequence is Uniprot Accession No. P043234. The human CD3-zeta polypeptide canonical sequence is Uniprot Accession No. P20963. The human TCR alpha chain canonical sequence is Uniprot Accession No. Q6ISU1. The human TCR beta chain C region canonical sequence is Uniprot Accession No. P01850, a human TCR beta chain V region sequence is P04435.

The human CD3-epsilon polypeptide canonical sequence is:

(SEQ ID NO: 4)
MQSGTHWRVLGLCLLSVGVWGQDGNEEMGGITQTPYKVSISGTTVILTCP
QYPGSEILWQHNDKNIGGDEDDKNIGSDEDHLSLKEFSELEQSGYYVCYP
RGSKPEDANFYLYLRARVCENCMEMDVMSVATIVIVDICITGGLLLLVYY
WSKNRKAKAKPVTRGAGAGGRQRGQNKERPPPVPNPDYEPIRKGQRDLYS
GLNQRRI.

The human CD3-gamma polypeptide canonical sequence is:

(SEQ ID NO: 5)
MEQGKGLAVLILAIILLQGTLAQSIKGNHLVKVYDYQEDGSVLLTCDAEA
KNITWFKDGKMIGFLTEDKKKWNLGSNAKDPRGMYQCKGSQNKSKPLQVY
YRMCQNCIELNAATISGFLFAEIVSIFVLAVGVYFIAGQDGVRQSRASDK
QTLLPNDQLYQPLKDREDDQYSHLQGNQLRRN.

The human CD3-delta polypeptide canonical sequence is:

(SEQ ID NO: 6)
MEHSTFLSGLVLATLLSQVSPFKIPIEELEDRVFVNCNTSITWVEGTVGT
LLSDITRLDLGKRILDPRGIYRCNGTDIYKDKESTVQVHYRMCQSCVELD
PATVAGIIVTDVIATLLLALGVFCFAGHETGRLSGAADTQALLRNDQVYQ
PLRDRDDAQYSHLGGNWARNKS.

The human CD3-zeta polypeptide canonical sequence is:

(SEQ ID NO: 7)
MKWKALFTAAILQAQLPITEAQSFGLLDPKLCYLLDGILFIYGVILTALF
LRVKFSRSADAPAYQQGQNQLYNELNLGRREEYDVLDKRRGRDPEMGGKP
QRRKNPQEGLYNELQKDKMAEAYSEIGMKGERRRGKGHDGLYQGLSTATK
DTYDALHMQALPPR.

The human TCR alpha chain canonical sequence is:

(SEQ ID NO: 8)
MAGTWLLLLLALGCPALPTGVGGTPFPSLAPPIMLLVDGKQQMVVVCLVL
DVAPPGLDSPIWFSAGNGSALDAFTYGPSPATDGTWTNLAHLSLPSEELA
SWEPLVCHTGPGAEGHSRSTQPMHLSGEASTARTCPQEPLRGTPGGALWL
GVLRLLLFKLLLFDLLLTCSCLCDPAGPLPSPATTTRLRALGSHRLHPAT
ETGGREATSSPRPQPRDRRWGDTPPGRKPGSPVWGEGSYLSSYPTCPAQA
WCSRSALRAPSSSLGAFFAGDLPPPLQAGAA.

The human TCR alpha chain C region canonical sequence is:

(SEQ ID NO: 9)
PNIQNPDPAVYQLRDSKSSDKSVCLFTDFDSQTNVSQSKDSDVYITDKTV
LDMRSMDFKSNSAVAWSNKSDFACANAFNNSIIPEDTFFPSPESSCDVKL
VEKSFETDTNLNFQNLSVIGFRILLLKVAGFNLLMTLRLWSS.

The human TCR alpha chain V region CTL-L17 canonical sequence is:

(SEQ ID NO: 10)
MAMLLGASVLILWLQPDWVNSQQKNDDQQVKQNSPSLSVQEGRISILNCD
YTNSMFDYFLWYKKYPAEGPTFLISISSIKDKNEDGRFTVFLNKSAKHLS
LHIVPSQPGDSAVYFCAAKGAGTASKLTFGTGTRLQVTL.

The human TCR beta chain C region canonical sequence is:

(SEQ ID NO: 11)
EDLNKVFPPEVAVFEPSEAEISHTQKATLVCLATGFFPDHVELSWWVNGK
EVHSGVSTDPQPLKEQPALNDSRYCLSSRLRVSATFWQNPRNHFRCQVQF
YGLSENDEWTQDRAKPVTQIVSAEAWGRADCGFTSVSYQQGVLSATILYE
ILLGKATLYAVLVSALVLMAMVKRKDF.

The human TCR beta chain V region CTL-L17 canonical sequence is:

(SEQ ID NO: 12)
MGTSLLCWMALCLLGADHADTGVSQNPRHNITKRGQNVTFRCDPISEHNR
LYWYRQTLGQGPEFLTYFQNEAQLEKSRLLSDRFSAERPKGSFSTLEIQR
TEQGDSAMYLCASSLAGLNQPQHFGDGTRLSIL.

The human TCR beta chain V region YT35 canonical sequence is:

(SEQ ID NO: 13)
MDSWTFCCVSLCILVAKHTDAGVIQSPRHEVTEMGQEVTLRCKPISGHNS
LFWYRQTMMRGLELLIYFNNNVPIDDSGMPEDRFSAKMPNASFSTLKIQP
SEPRDSAVYFCASSFSTCSANYGYTFGSGTRLTVV.

Generation of TFPs from TCR Domains and scFvs

The mesothelin scFvs are recombinantly linked to CD3-epsilon or other TCR subunits using a linker sequence, such as G₄S, (G₄S)₂ (G₄S)₃ or (G₄S)₄. Various linkers and scFv configurations are utilized. TCR alpha and TCR beta chains were used for generation of TFPs either as full length polypeptides or only their constant domains. Any variable sequence of TCR alpha and TCR beta chains is allowed for making TFPs.

### TFP Expression Vectors

Expression vectors are provided that include: a promoter (Cytomegalovirus (CMV) enhancer-promoter), a signal sequence to enable secretion, a polyadenylation signal and transcription terminator (Bovine Growth Hormone (BGH) gene), an element allowing episomal replication and replication in prokaryotes (e.g., SV40 origin and ColE1 or others known in the art) and elements to allow selection (ampicillin resistance gene and zeocin marker).

Preferably, the TFP-encoding nucleic acid construct is cloned into a lentiviral expression vector and expression validated based on the quantity and quality of the effector T cell response of TFP.mesothelin-transduced T cells (“mesothelin.TFP” or “mesothelin.TFP T cells” or “TFP.mesothelin” or “TFP.mesothelin T cells”) in response to mesothelin+ target cells. Effector T cell responses include, but are not limited to, cellular expansion, proliferation, doubling, cytokine production and target cell lysis or cytolytic activity (i.e., degranulation).

The TFP.mesothelin lentiviral transfer vectors are used to produce the genomic material packaged into the VSV-G pseudotyped lentiviral particles. Lentiviral transfer vector DNA is mixed with the three packaging components of VSV-G, gag/pol and rev in combination with Lipofectamine® reagent to transfect them together into HEK-293 (embryonic kidney, ATCC® CRL-1573™) cells. After 24 and 48 hours, the media is collected, filtered and concentrated by ultracentrifugation. The resulting viral preparation is stored at −80° C. The number of transducing units is determined by titration on Sup-T1 (T cell lymphoblastic lymphoma, ATCC® CRL-1942™) cells. Redirected TFP.mesothelin T cells are produced by activating fresh naïve T cells with, e.g., anti-CD3 anti-CD28 beads for 24 hrs and then adding the appropriate number of transducing units to obtain the desired percentage of transduced T cells. These modified T cells are allowed to expand until they become rested and come down in size at which point they are cryopreserved for later analysis. The cell numbers and sizes are measured using a Coulter Multisizer™ III. Before cryopreserving, the percentage of cells transduced (expressing TFP.mesothelin on the cell surface) and the relative fluorescence intensity of that expression are determined by flow cytometric analysis. From the histogram plots, the relative expression levels of the TFPs are examined by comparing percentage transduced with their relative fluorescent intensity.

In some embodiments multiple TFPs are introduced by T cell transduction with multiple viral vectors.

### Evaluating Cytolytic Activity, Proliferation Capabilities and Cytokine Secretion of Humanized TFP Redirected T Cells

The functional abilities of TFP.mesothelin T cells to produce cell-surface expressed TFPs, and to kill target tumor cells, proliferate and secrete cytokines are determined using assays known in the art.

Human peripheral blood mononuclear cells (PBMCs, e.g., blood from a normal apheresed donor whose naïve T cells are obtained by negative selection for T cells, CD4+ and CD8+ lymphocytes) are treated with human interleukin-2 (IL-2) then activated with anti-CD3x anti-CD28 beads, e.g., in 10% RPMI at 37° C., 5% CO₂ prior to transduction with the TFP-encoding lentiviral vectors. Flow cytometry assays are used to confirm cell surface presence of a TFP, such as by an anti-FLAG antibody or an anti-murine variable domain antibody. Cytokine (e.g., IFN-γ) production is measured using ELISA or other assays.

### Example 3: Anti-MSLN TFP T Cells have a Distinctive Phenotype Compared to CAR-T Cells

Analysis of immune cell states is paramount to our understanding of the pathogenesis of a broad range of human diseases. Immunologists rely on fluorescence cytometry for cellular analysis, and while detection of 8 markers is now well established, the overlap of fluorescent signals limits efficiency. Mass cytometry or CyTOF (Cytometry by Time-Of-Flight) is a technology for multiparameter single cell analysis that overcomes many limitations of fluorescence-based flow cytometry and can routinely detect as many as 40 markers per sample (see, e.g., Yao et al., J. Immunol. Methods (2014) Dec. 15; 415:1-5).

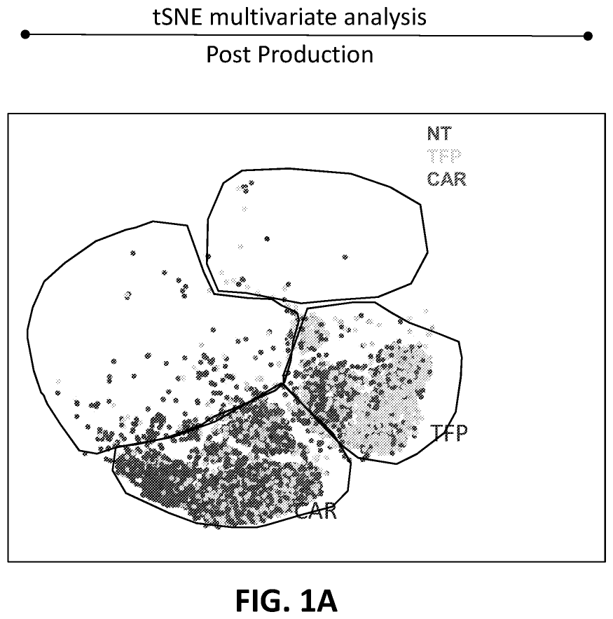

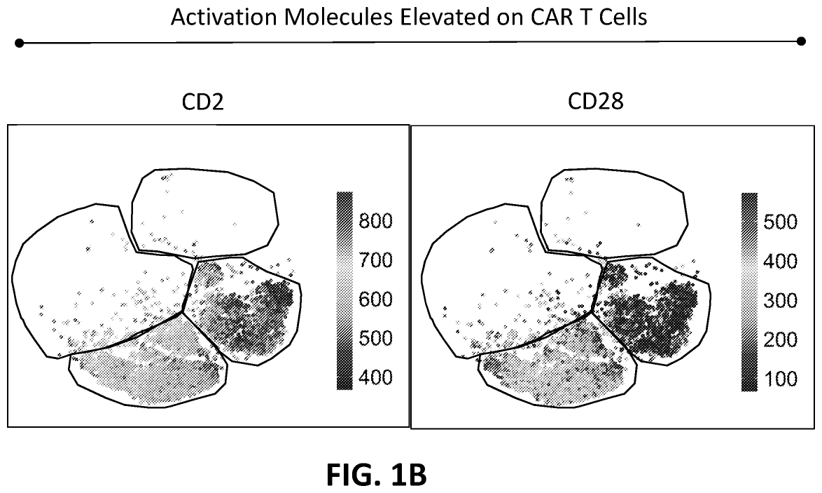

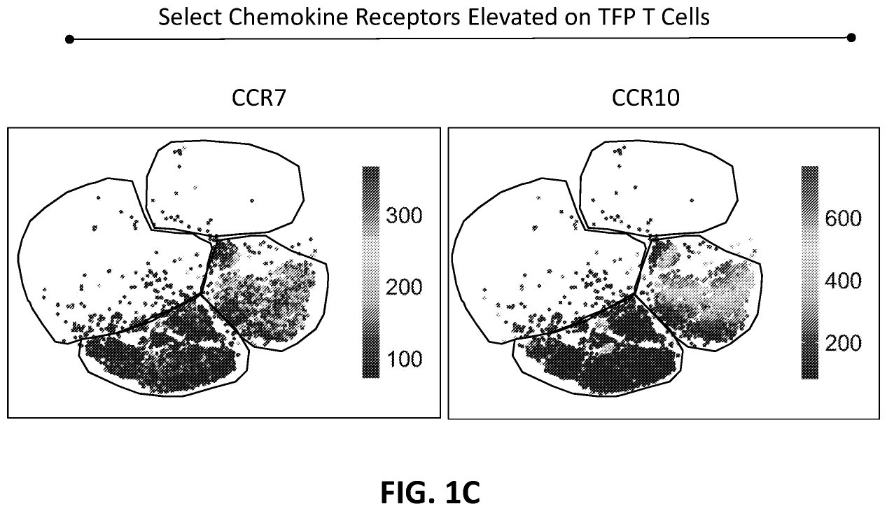

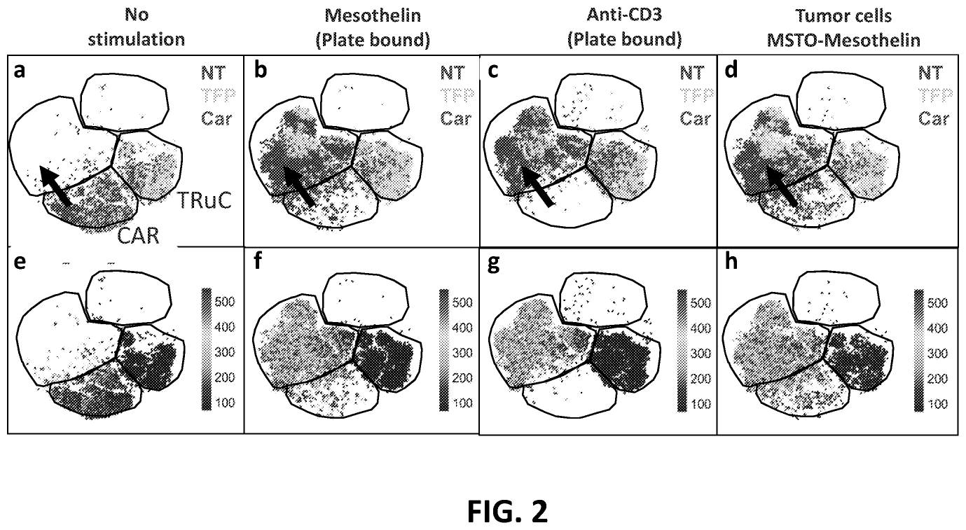

Mesothelin binder SD1 TFP-T cells or SD1-BBζ CAR-T cells were thawed and cultured overnight as described above. For CyTOF analysis, SD1 TFP T cells and SD1-BBζ CAR (CAR) were labelled with 36 cell surface expressed molecules (CD57, CCR10, CXCR3, CD161, CD69, CD44, CD27, CD95, CD152, CD2, CD62L, CD3, CD137, LAG3, CCR4, OX40, CD16, CD279, CD127, CD11a, CD5, CCR5, CD4, CD8A, CD28, ICOS, CD49D, CD7, TIM3, CD45RO, CD197, CD25, CD40, MH1, CD96, HLADR). Non-biased, multiparametric analysis was conducted using t-distributed stochastic neighbor embedding (tSNE), implemented in R. Data are representative of at least 3 replicates, stained using a barcoding approach (labelling CD45). SD1 TFP-T cells show a unique phenotype, characterized by lower activation molecule (FIG. 1B) and higher chemokine receptor expression (FIG. 1C).

### Example 4. Anti-MSLN TFP T Cells Demonstrate Dose-Dependent Activity and Persistence

The efficacy of treatment with human TFP.mesothelin T cells can be tested in immune compromised mouse models bearing subcutaneous solid tumors derived from human mesothelin-expressing ALL, CLL, NHL, or MSTO human cell lines. Tumor shrinkage in response to treatment with human TFP.mesothelin T cells can be either assessed by caliper measurement of tumor size or by following the intensity of a green fluorescence protein (GFP) signal emitted by GFP-expressing tumor cells.

Primary human solid tumor cells can be grown in immune compromised mice without having to culture them in vitro. Exemplary solid cancer cells include solid tumor cell lines, such as provided in The Cancer Genome Atlas (TCGA) and/or the Broad Cancer Cell Line Encyclopedia (CCLE, see Barretina et al., Nature 483:603 (2012)). Exemplary solid cancer cells include primary tumor cells isolated from mesothelioma, renal cell carcinoma, stomach cancer, breast cancer, lung cancer, ovarian cancer, prostate cancer, colon cancer, cervical cancer, brain cancer, liver cancer, pancreatic cancer, kidney, endometrial, or stomach cancer. In some embodiments, the cancer to be treated is selected from the group consisting of mesotheliomas, papillary serous ovarian adenocarcinomas, clear cell ovarian carcinomas, mixed Mullerian ovarian carcinomas, endometroid mucinous ovarian carcinomas, pancreatic adenocarcinomas, ductal pancreatic adenocarcinomas, uterine serous carcinomas, lung adenocarcinomas, extrahepatic bile duct carcinomas, gastric adenocarcinomas, esophageal adenocarcinomas, colorectal adenocarcinomas and breast adenocarcinomas. These mice can be used to test the efficacy of TFP.mesothelin T cells in the human tumor xenograft models (see, e.g., Morton et al., Nat. Procol. 2:247 (2007)). Following an implant or injection of 1×10⁶-1×10⁷ primary cells (collagenase-treated bulk tumor suspensions in EC matrix material) or tumor fragments (primary tumor fragments in EC matrix material) subcutaneously, tumors are allowed to grow to 200-500 mm³ prior to initiation of treatment.

One such experiment was performed to test the efficacy of MSLN-specific single domain antibody SD1 activity in vivo in a mesothelioma xenograft mouse model as described above. MSTO^{MSLN} tumor cells were inoculated at 1×10⁶ cells per mouse, subcutaneously, as a 1:1 ratio with Matrigel®, at Day −12. Tumor volume was monitored by caliper measurement twice weekly. Fourteen days after tumor injection, when tumor volume was approximately 200 mm³, 1×10⁷ T cells were injected intravenously into each animal. Ex vivo analysis of the immune response was performed.

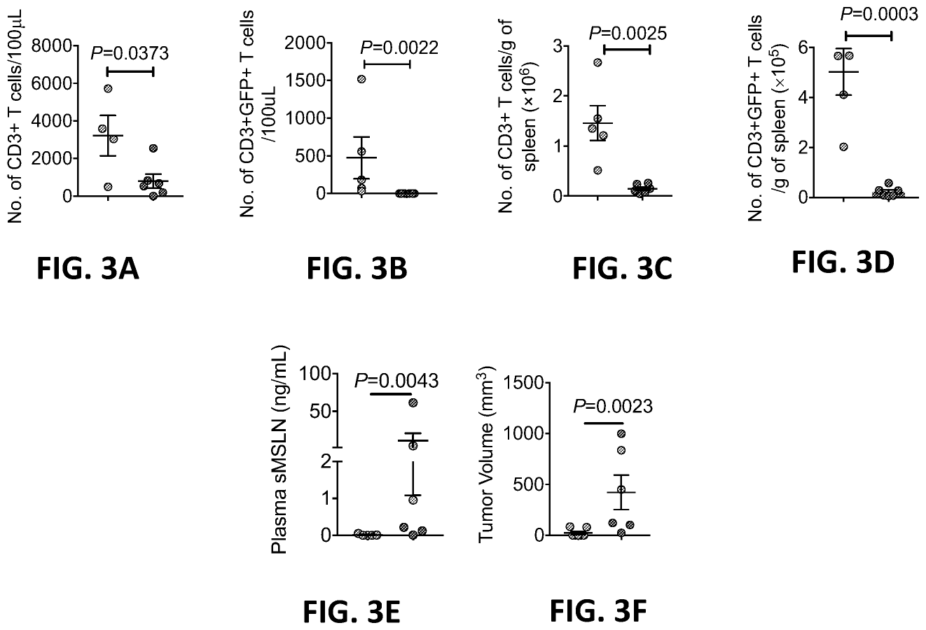

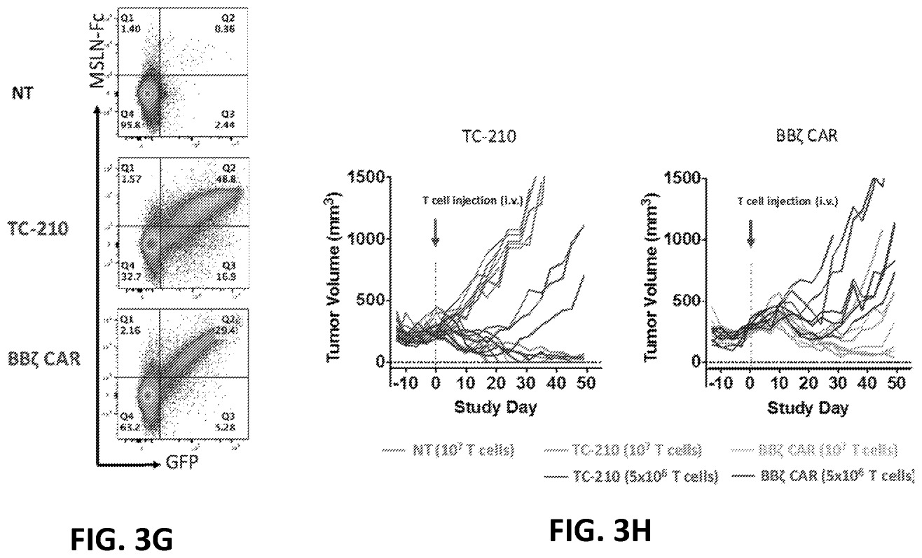

Ex vivo analysis of total CD3+ T cells (FIG. 3A) and transduced (FIG. 3B) T cells in the blood of NSG mice xenografted s.c. with MSTO^{MSLN} cells 56 days after inoculation showed increased numbers of circulating SD1 TFP-T cells. A similar phenomenon was observed in total CD3+ cells (FIG. 3C) and SD1 TFP-T cells (FIG. 3D) in the spleen. Interestingly, plasma MSLN levels (FIG. 3E) correlated with tumor volume (FIG. 3F) on day 56.

### Example 5. Day 7 Analysis Shows Tumor Control & Increased SD1 TFP-T Cells in Circulation

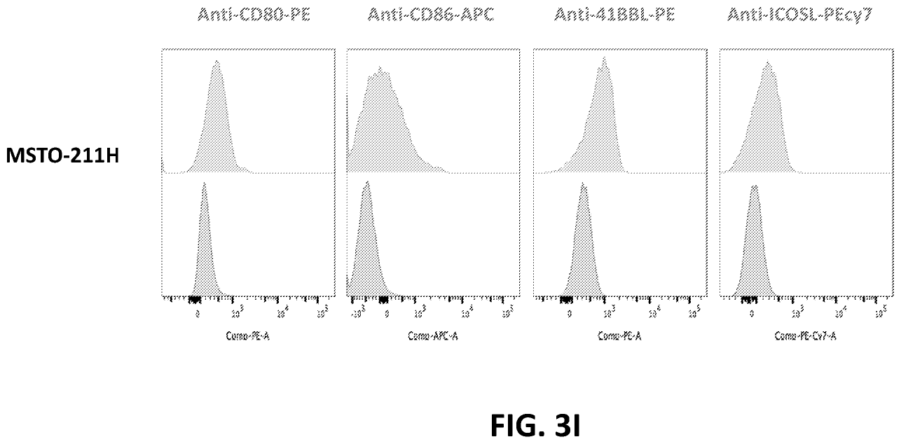

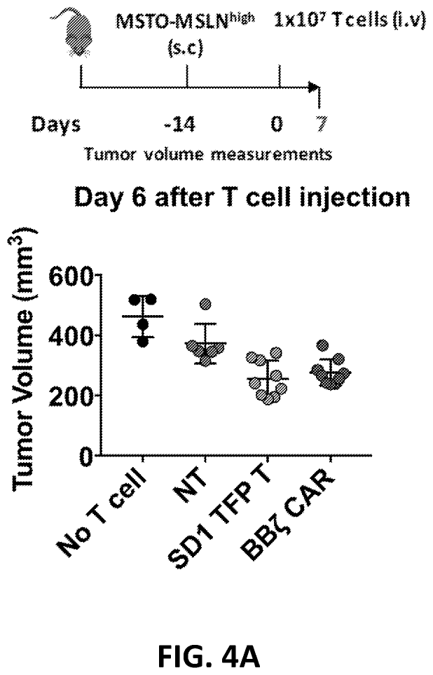

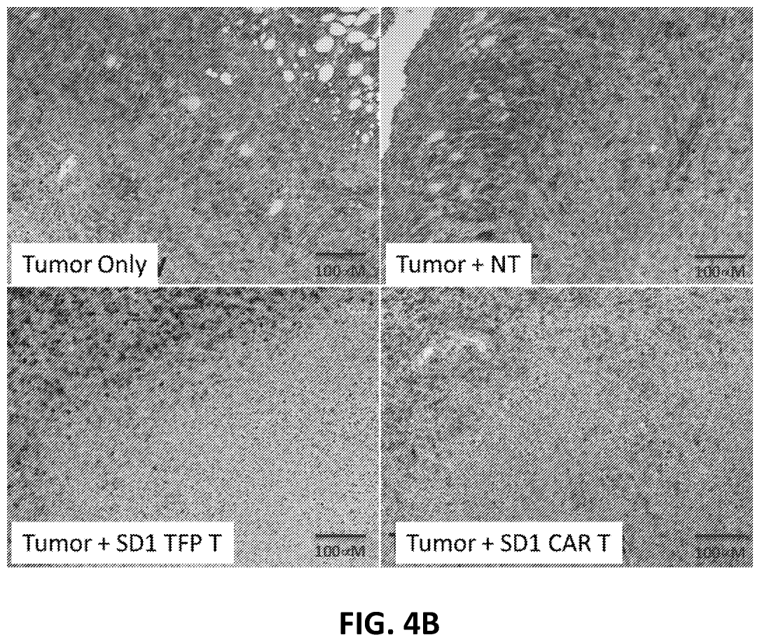

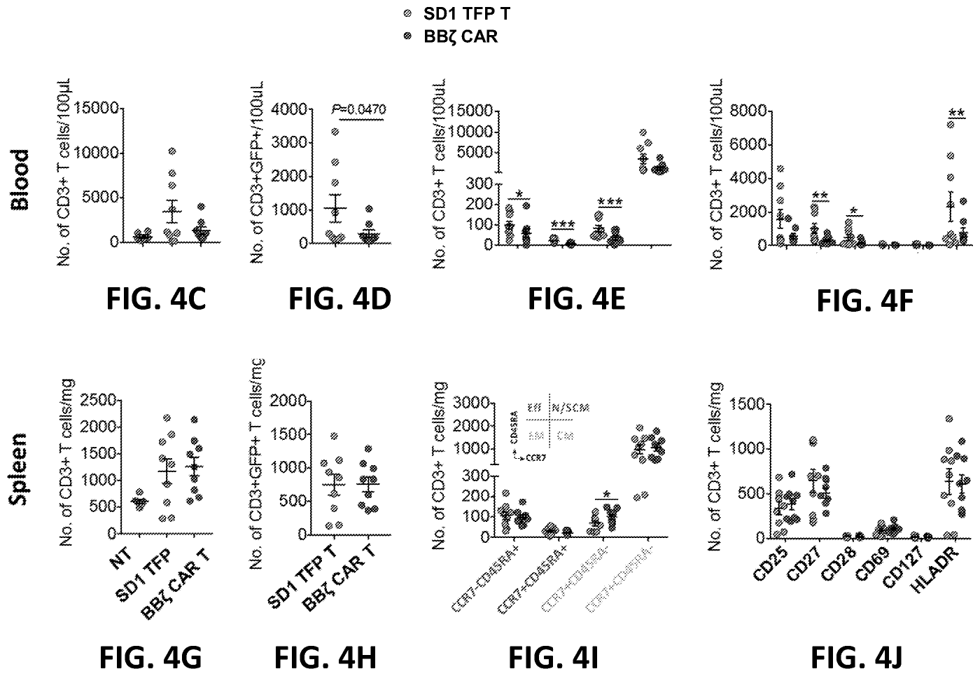

MSTO^{MSLN} tumor cells were administered as described in Example 4. Four groups of animals were either treated with no T cells (n=4), NT T cells (n=5), SD1 TFP T cells (n=9) or SD1-BBζ CAR-T cells (n=9) On day 6 the tumor burden was measured (FIG. 4A). On day 7, animals were sacrificed an the tumors processed and stained with anti-CD3 (maroon) and anti-mesothelin (brown) antibodies, with a hematoxylin counter stain. Results are shown in FIG. 4B. The top left panel shows a section of tumor from animals treated with no T cells, top right with non-transduced (NT) T cells, bottom left with SD1 TFP T cells, and bottom right with SD1 CAR-T cells. As can be seen in the Figure, the untreated or non-transduced T cell treated tumor cells show staining with predominantly anti-MSLN antibodies, whereas the bottom two panels from mice transduced with the SD1 TFP or CAR-T cells have a much higher amount of CD3+ cells and a lower amount of MSLN+ cells.

Next was measured the amount of either circulating T cells from blood (FIG. 4C-F) or spleen (FIG. 4G-J). Using flow cytometry, total circulating T cells (CD3+; 4C) and SD1 TFP T cells (CD3+GFP+; 4D) as well as their phenotype (FIG. 4E) and activation (FIG. 4F) status was examined. This was compared to the total T cells (FIG. 4G) and SD1 TFP T cell number (FIG. 4H), and their phenotype (FIG. 4I) and activation (FIG. 4J) profile in the spleen. In FIGS. 4E-F and 4I-J, for each x-axis label the dta are represented pairwise with dots representing SD1 TFP T cells on the left and dots representing SD1 CAR-T cells on the right.

The overall data show reductions in tumor MSLN expression and increased numbers of circulating SD1 TFP T cells (compared to CAR-T cells) in treated animals.

### Example 6. SD1 TFP T Cells Release Lower Levels of Cytokines than CAR-T Cells In Vivo

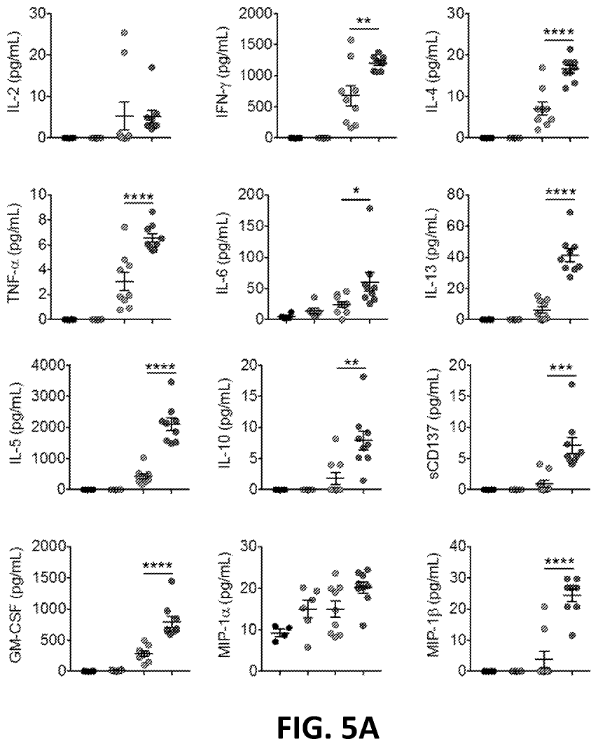

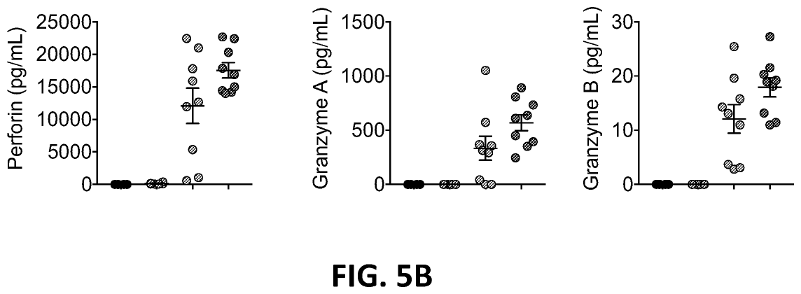

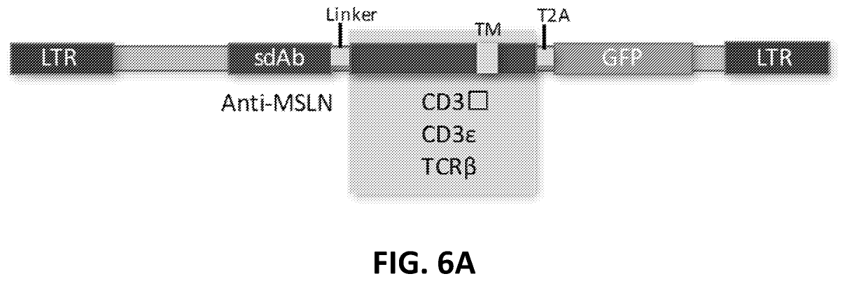

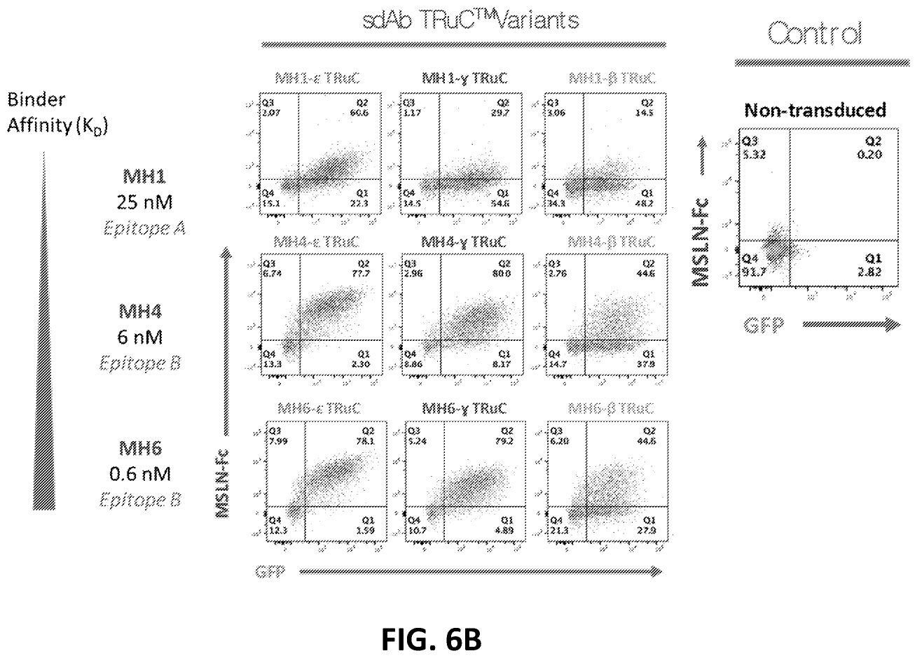

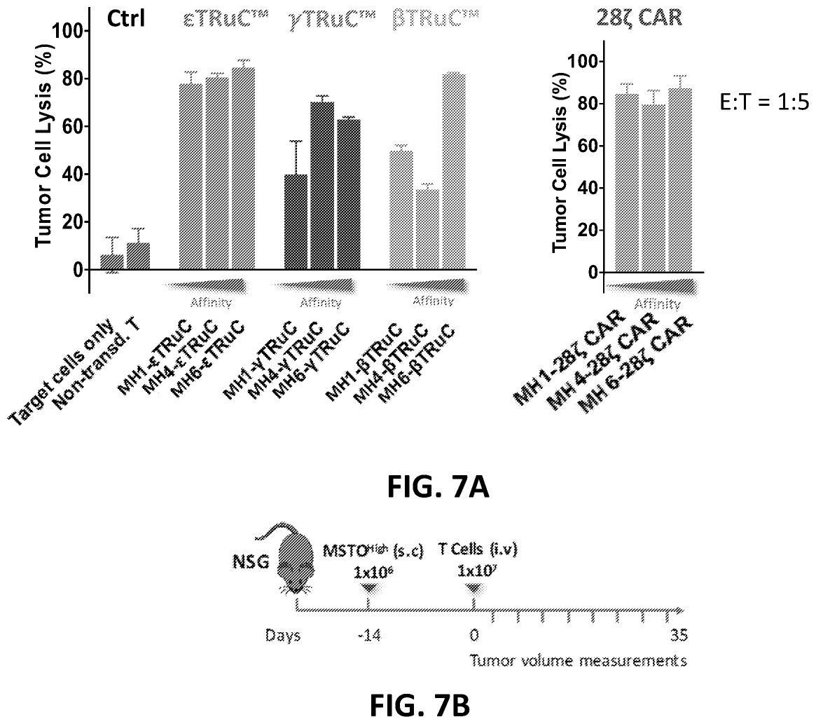

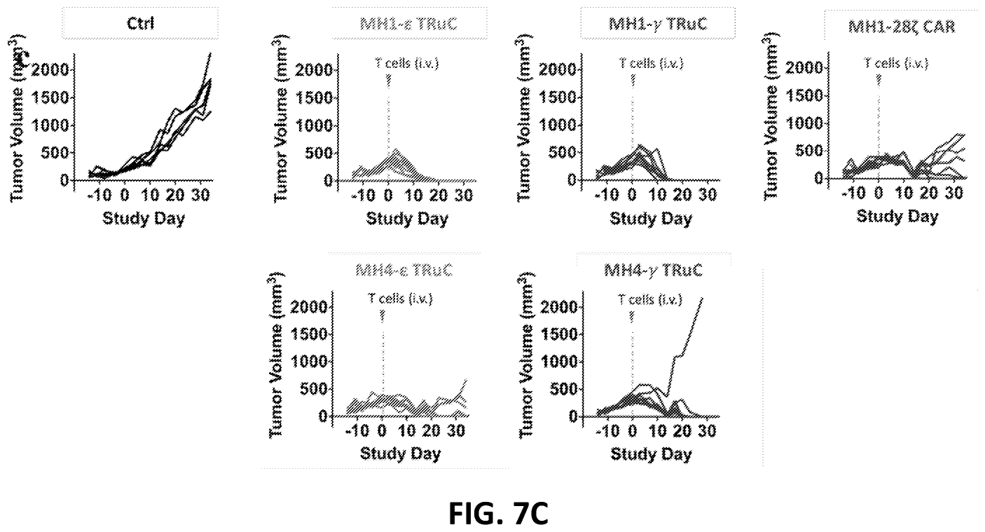

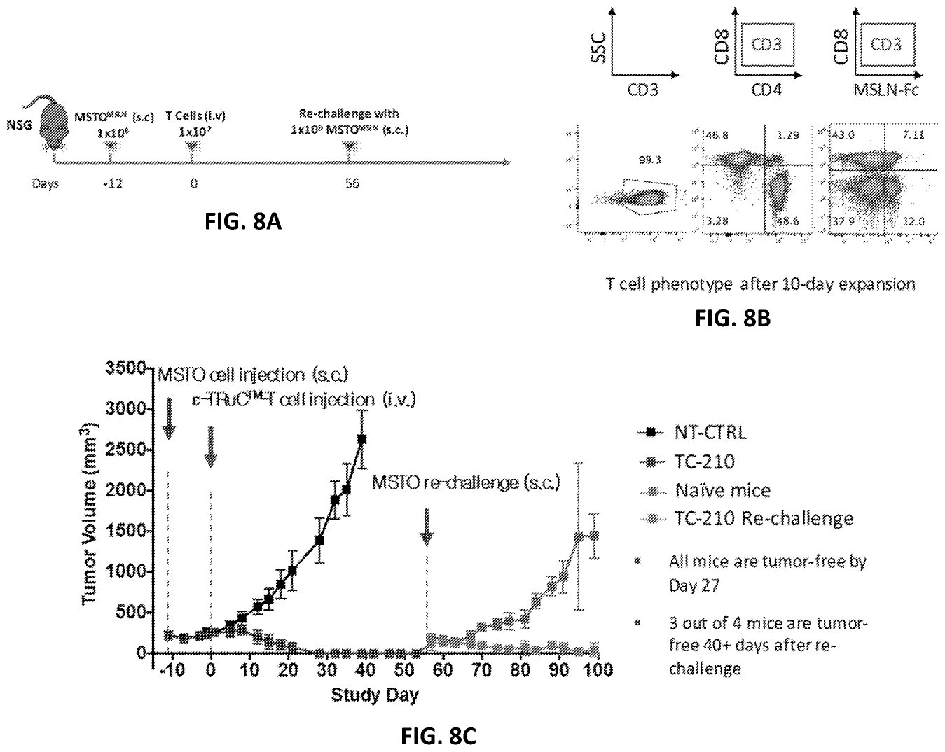

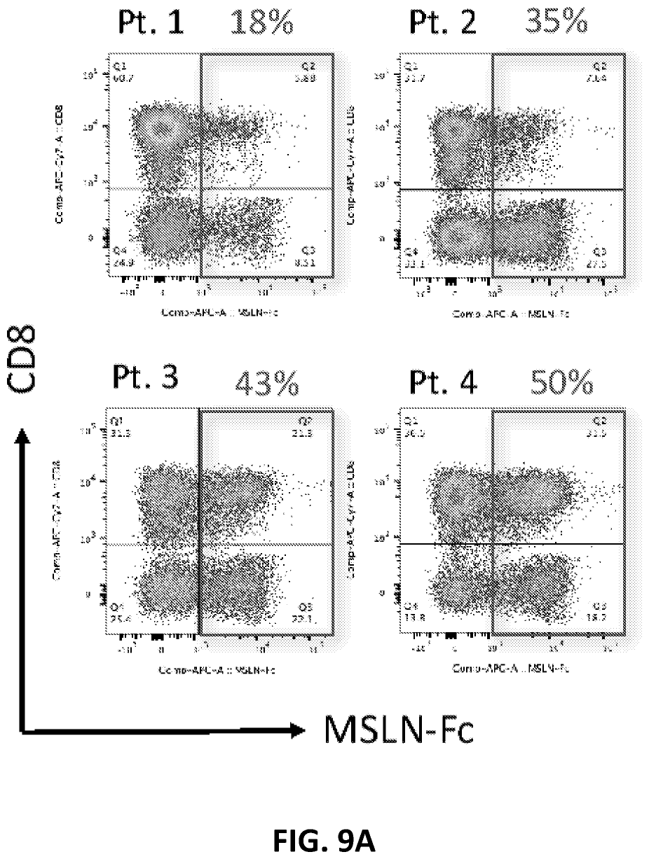

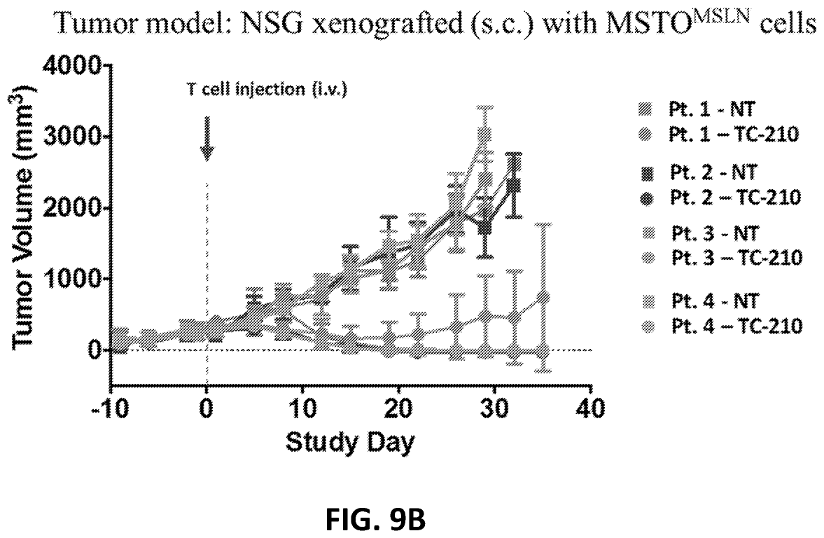

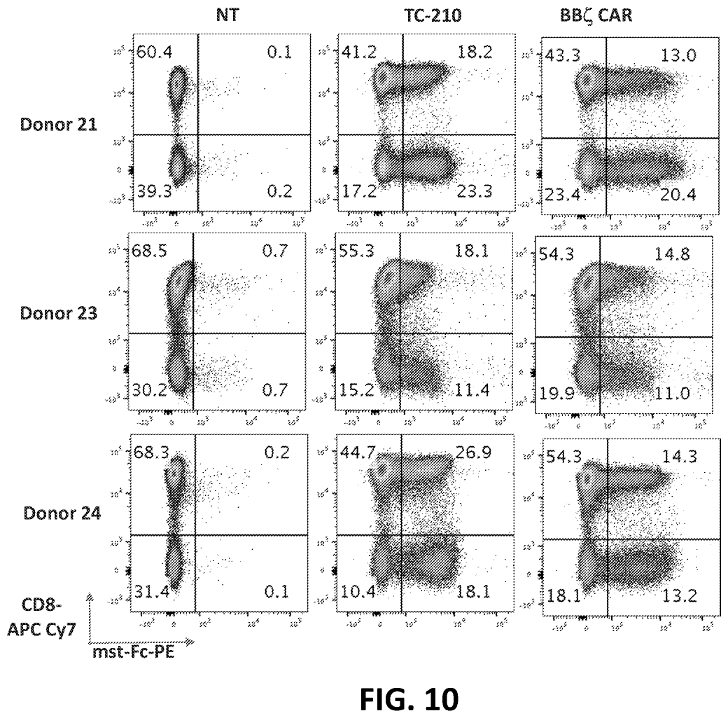

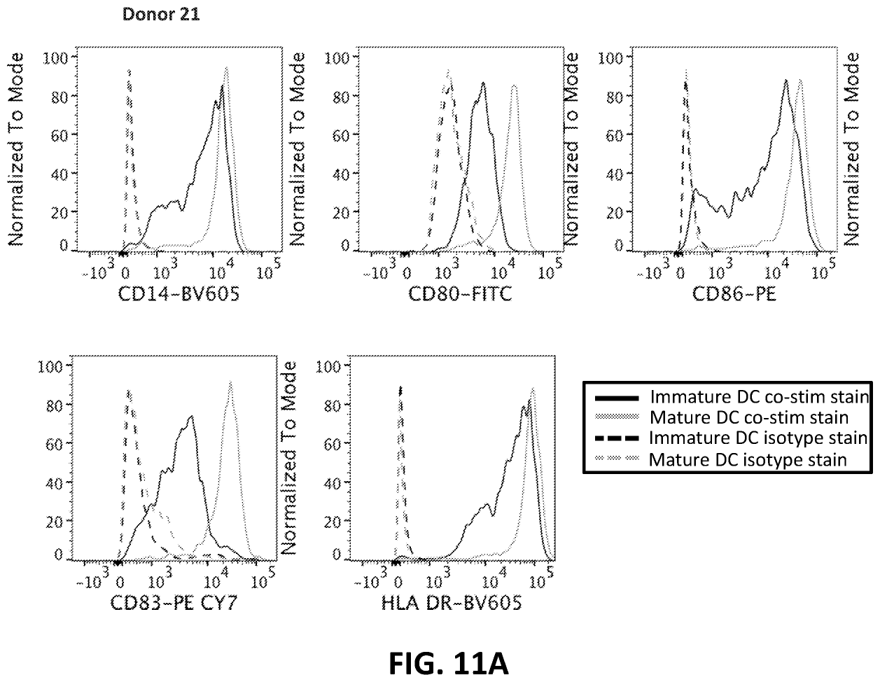

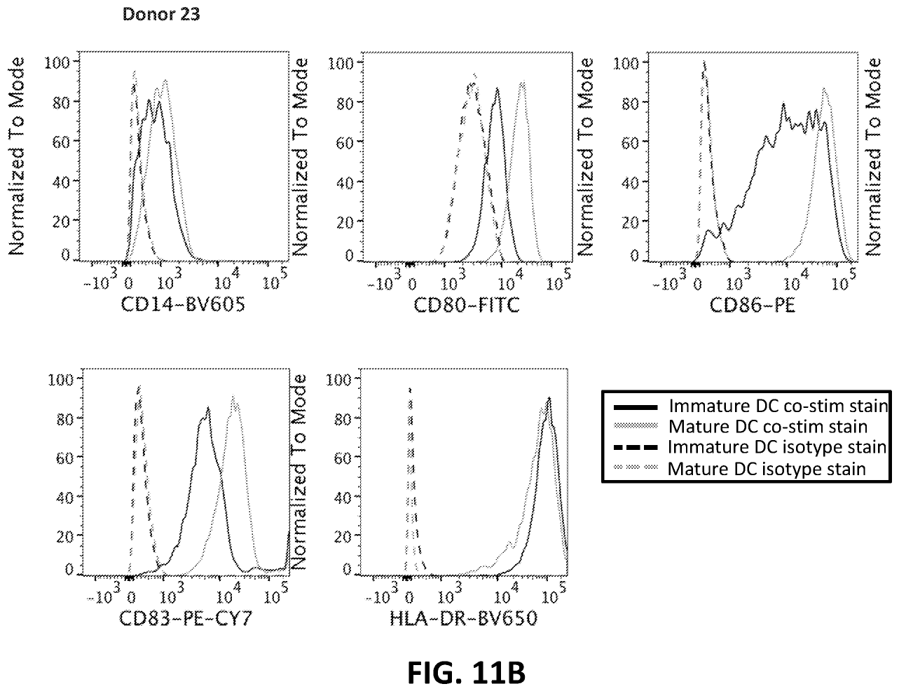

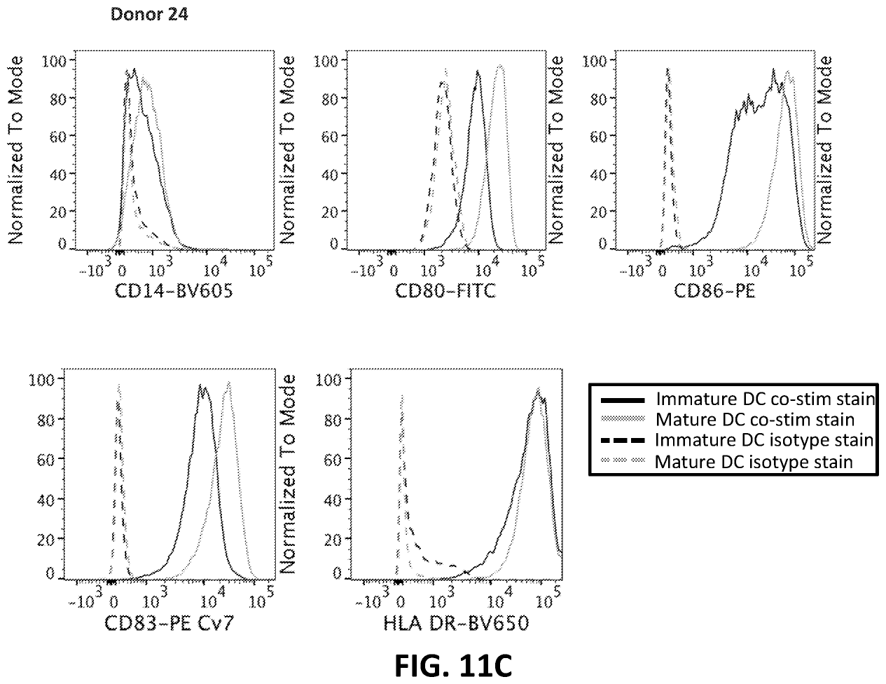

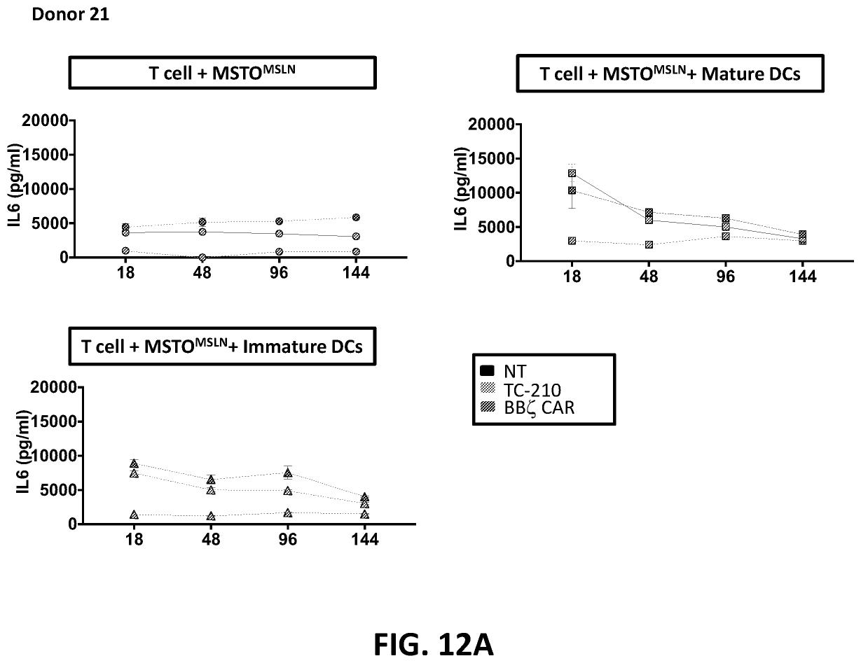

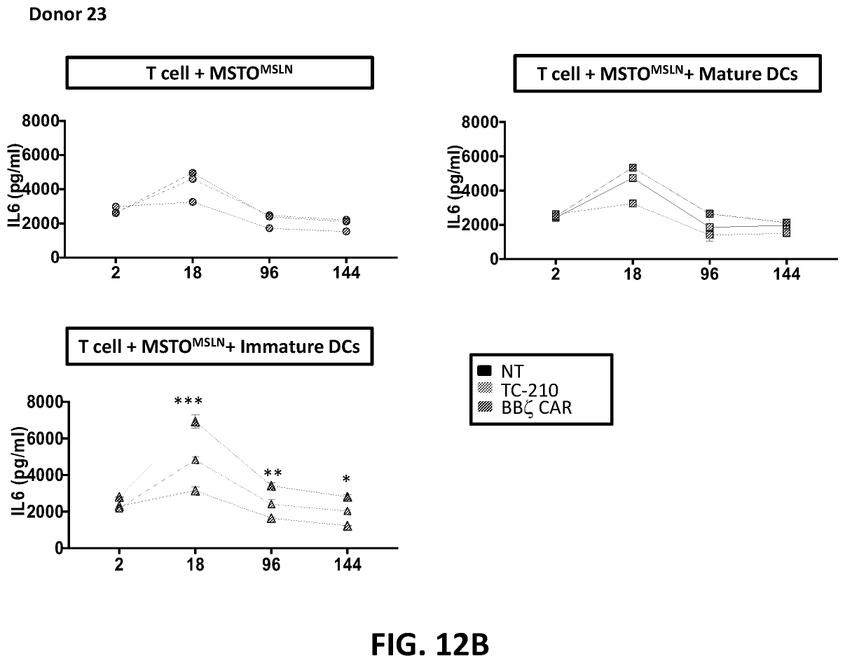

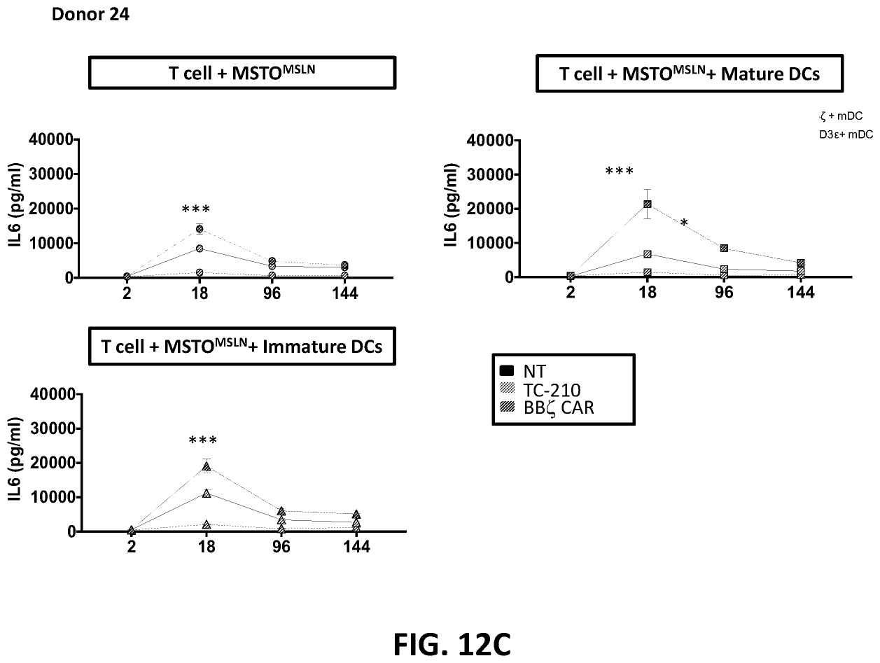

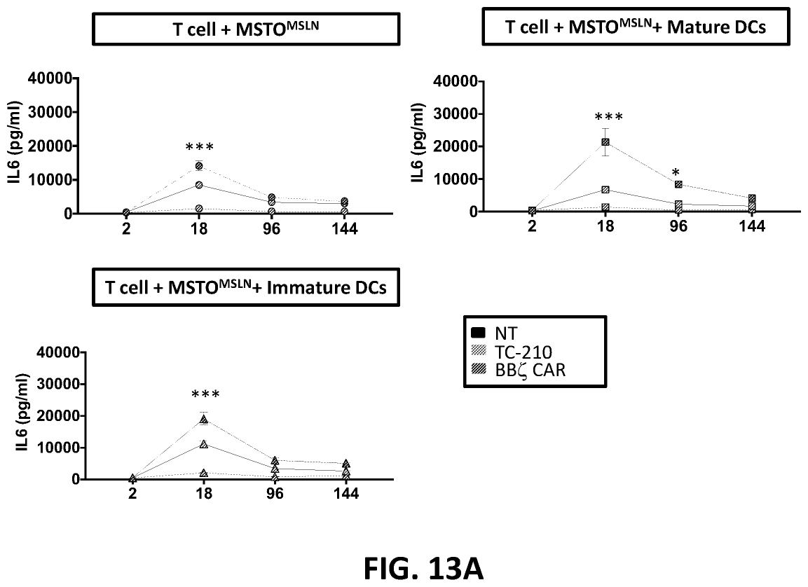

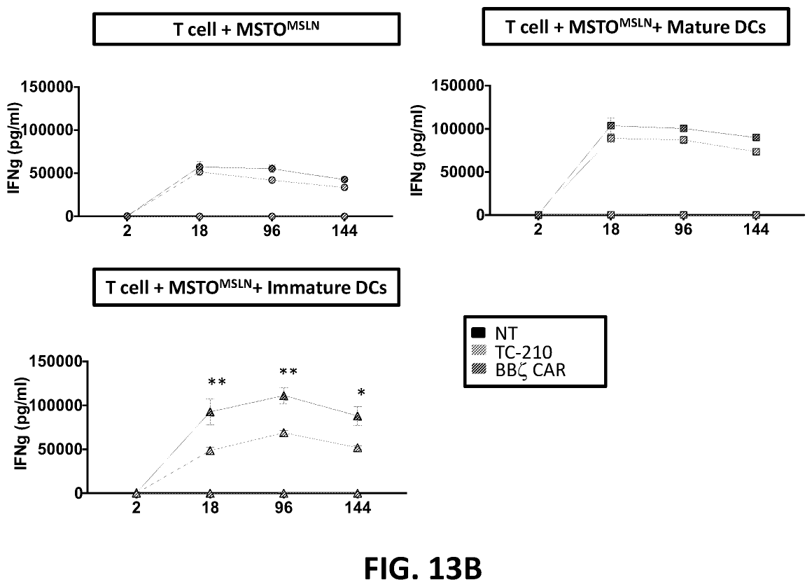

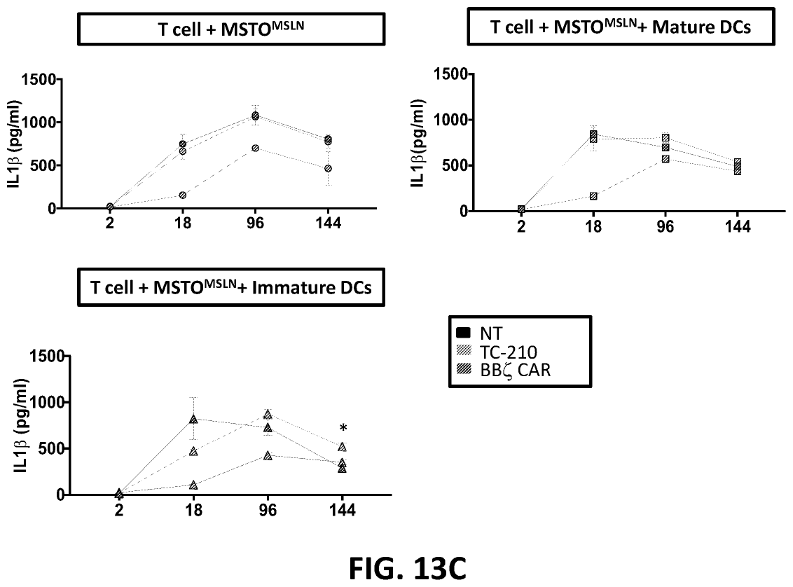

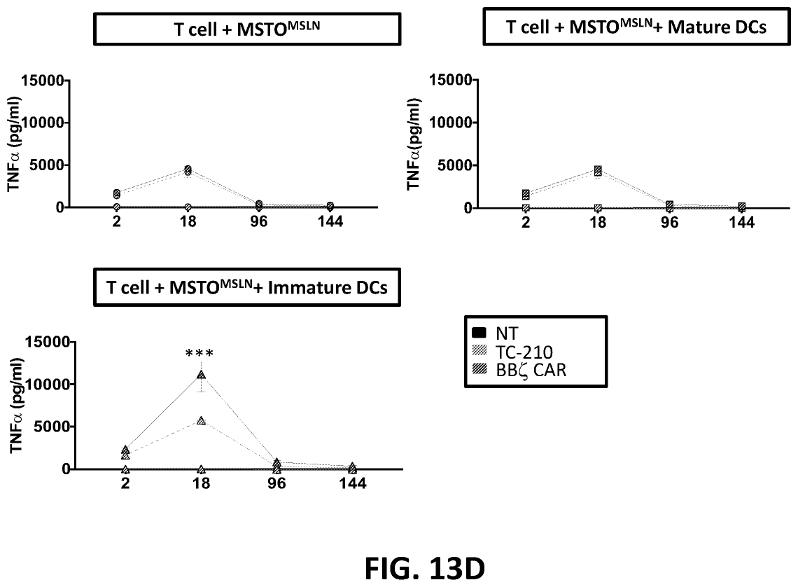

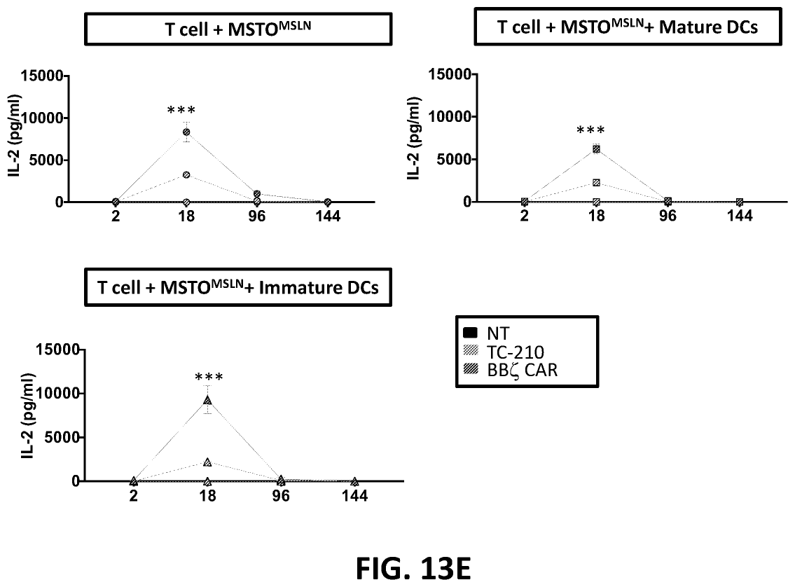

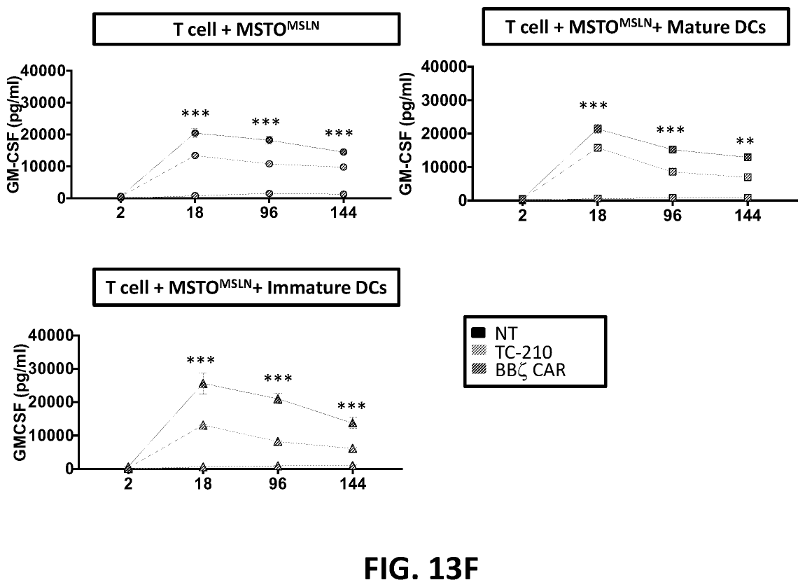

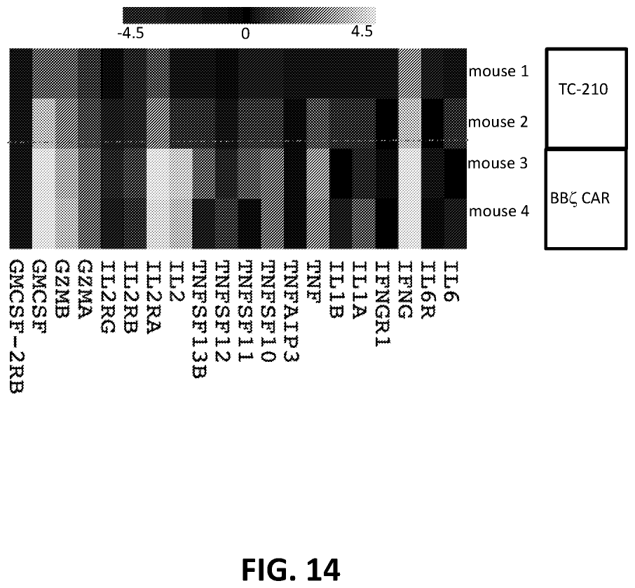

The Day 7 post-treatment plasma of the animals described in Example 5 was analyzed for levels of cytokines (FIG. 5A) and cytotoxic payload proteins (FIG. 5B). Analytes were measured using the human CD8+ T cell Magnetic Bead Panel (Millipore). In each panel, the x-axis represents, from left to right, plasma from untreated mice, mice treated with nontransduced T cells (NT), Mice treated with SD1 TFP T cells, and mice treated with SD1 CAR-T cells. As shown in FIG. 5A, SD1 TFP T cells result in lower levels of plasma cytokines than SD1 CAR-T cells, including IL-2, IFN-γ, IL-4, IL-5, IL-10, sCD137, TNF-α, IL-6, IL-13, GM-CSF, MIP-1α, and MIP-1β. As shown in FIG. 5B, levels of cytotoxic proteins perforin, granzyme A, and granzyme B, were comparable between the two groups. The data demonstrate that in NSG models SD1 TFP T cell treatment results in lower levels of plasma cytokines than treatment with CAR-T cells with the same SD1 binder.

### Example 7. Gene Expression Analysis in MSLN-Specific TFP T and CAR-T Cells Upon Antigen Stimulation

### Overview

Assessment of genetic pathway of T cell interaction with antigen-specific tumor cells can reveal the unique property of TRuC T cells in comparison to CAR-T cells. These differences between the gene expression of antigen-stimulated T cells could potentially explain the comparable tumor-specific lysis and persistence of TRuCs in in vivo tumor models and aid in bringing about changes in the platform development to harness TRuC T cell activity in patients. nCounter analysis (Nanostring), is a powerful digital detection system that uses hybridization-based technology. This tool is capable of multiplexing (more than 500 immunology genes), and profiling all of the individual mRNA gene transcripts directly without the requirement of amplification step. Data generated using this platform is highly accurate in profiling changes in gene expression before and after antigen-stimulation in T cells. In this study, we reveal that TRuC T cells recruit Killer cell lectin like receptor family members and chemokines such as CXCL13 and cytokines such as IL1A, IL7R, IL6R to proliferate and persist T cells when compared to CAR-T cells upon 24-hour stimulation with plate-bound antigen.

### Study Design

The study consists of four groups various TRuC and CAR format namely CD3 epsilon (CD3ε) TRuC, CD28zeta CAR, 41BBzeta CAR and NT.

### Materials and Methods

Lentivirus Generation

The lentivirus was prepared by transient transfection of 293TN Producer Cell Line (System Biosciences, Inc., LV900A-1) TRUC and CAR constructs were generated using MH1 scFv fused to CD3 epsilon chain or CD28-CD3 zeta chain or 41BBz-CD3 zeta chain.

T Cells Isolation and Lentivirus Transduction

CD4 and CD8 T cells were purified from Leukopack sample (Hemacare, (Donor 14 Lot #W313716041176, Donor 15 Lot #W313717041459, Donor 17 Lot #W313717041771). Leukaphresis sample was subjected to CD4 and CD8 T cell enrichment using CD4 (Miltenyi, Catalog #130-030-401, Lot #5160914085) and CD8 MACS beads (Miltenyi, Catalog #130-030-801, Lot #5160830314) using automated CliniMACS Prodigy automated system (Miltenyi) according to manufacturer's instructions.

T cells were activated using Dyna beads (Gibco, Catalog #00415447, Lot #1785079) at 1:1 ratio and were maintained in AimV plus AlbuMAX media (Gibco, Catalog #31035-025) in 5% hAB serum (Gemini Products, Catalog #100-318, lot #H605oo1), and 1% antibiotics (Gibco, Catalog #15240-062, Lot #1734036) in presence of 300 IU/ml IL-2 (Peprotech, Catalog #200-02, lot #051512). Dynabead activated T cells were transduced with lentivirus at 1 MOI respectively in presence of polybrene (5ug/ml) (Millipore, Catalog #TR-1003-G) and spinoculation at 100G for 100 minutes. A total of two rounds of transductions were performed at 24 hour and 48-hour post transduction.

Transduction Efficiency and Activation Determination:

Transduction efficiency was determined by flow cytometry. T cells were stained using anti-CD3 APC (Clone, UCHT1. BD Biosciences Catalog #340440, Lot #6005787), anti-CD4-Pacific blue (CloneRPAT4, Biolegend, Catalog #300521, Lot #B231611), anti-CD8-APCCY7 (Clone SK1, BD Biosciences, Catalog #557834, Lot #, 6082865), mesothelin antigen (Acro bioscience, Catalog #904x-7289F1-E7, Lot #904x-3AOS1-4N), CD69-AF 700 (Clone FN50, Catalog #560739, Lot #7051802), Zenon R-Phycoerythrin Human IgG Labeling Kit (Thermofischer Scientific, Catalog number #Z25408, Lot #1863290) and isotype controls, APC Mouse IgG1, k Isotype Control (Clone X40, BD Biosciences, Catalog #340442) Pacific Blue isotype control (Clone MOPC-21, BD Biosciences, Catalog #558120), APCCY7 IgG1 isotype control (BD Biosciences, Catalog #557873), AF700 IgG1 isotype control (Clone 27-35, BD Biosciences, Catalog #560543). Cells were analyzed using BD-LSRII Fortessa X20.

T Cell Activation Using Plate Bound Antigen:

1.5 mg/ml of mesothelin antigen (Acro bioscience, Catalog #904x-7289F1-E7, Lot #904x-3AOS1-4N) was coated on 96-well flat bottomed high binding plate overnight at 4 C. Next day, plate was washed twice with 1×PBS and blocked with 1% BSA for 30 mins at 4 C. Plates were washed with 1×PBS and 100,000 TRuC or CAR-T cells were plated per well and placed at 37 C incubator. 24 hours after plating T cells, the cells were harvested and placed in RLT lysis buffer (Qiagen, Catalog #79216) (10,000 cells lysed in 1 ul of buffer) and one representative well from each condition was chosen for flow analysis.

Bioinfomatics Data Analysis:

The raw count for each transduction condition was normalized using the endogenous control (up to 15 genes used). Ratio of genes upregulated or down regulated after co-culture and before co-culture was calculated for each transduction condition. Statistical t-test was used to calculate p-value of ratio for average of each donor in TRuC and CARs. R-program based nSolver V3 was used to score genes that are at least log 1.5 upregulated or down regulated specifically in TRuCs compared to CARs. Heatmaps were generated using genes that were either significantly changed compared to CARs or greater or lesser than log 1.5 fold difference using the nSolver V3 software.

Pathway analysis was performed by entering the significant upregulated or down regulated TRuC genes list into www.reactome.org software and genes based on FDR hits for each pathway was plotted in a graphical representation.

### Results

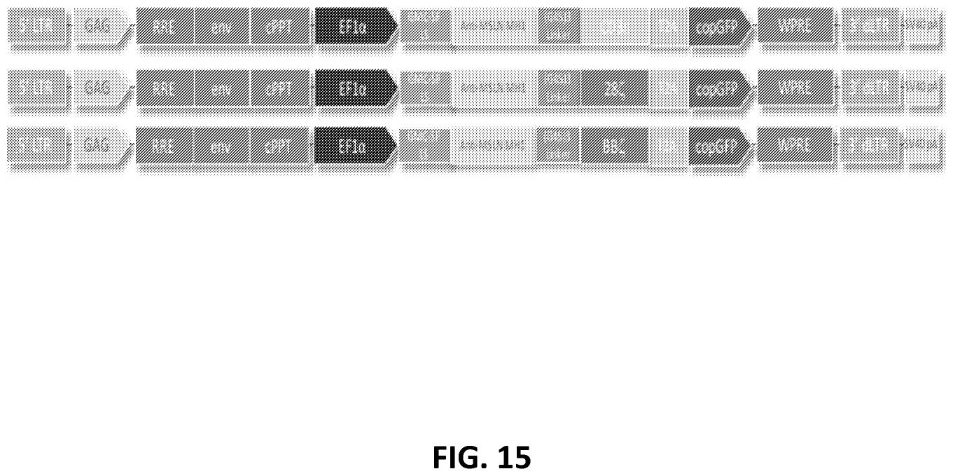

T cells were enriched for CD4 and CD8 T cells and transduced with indicated lentivirus vectors (FIG. 15). Lentivirus vector for each transgene contained a T2A cleavage site which allowed us to evaluate transduction efficiency and surface expression of these novel transgenes.

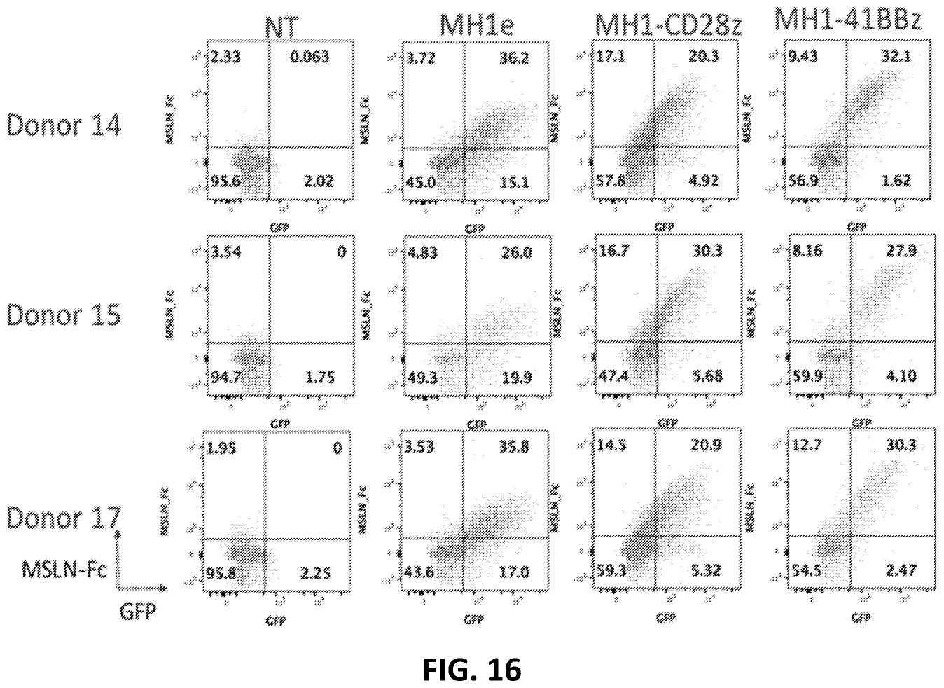

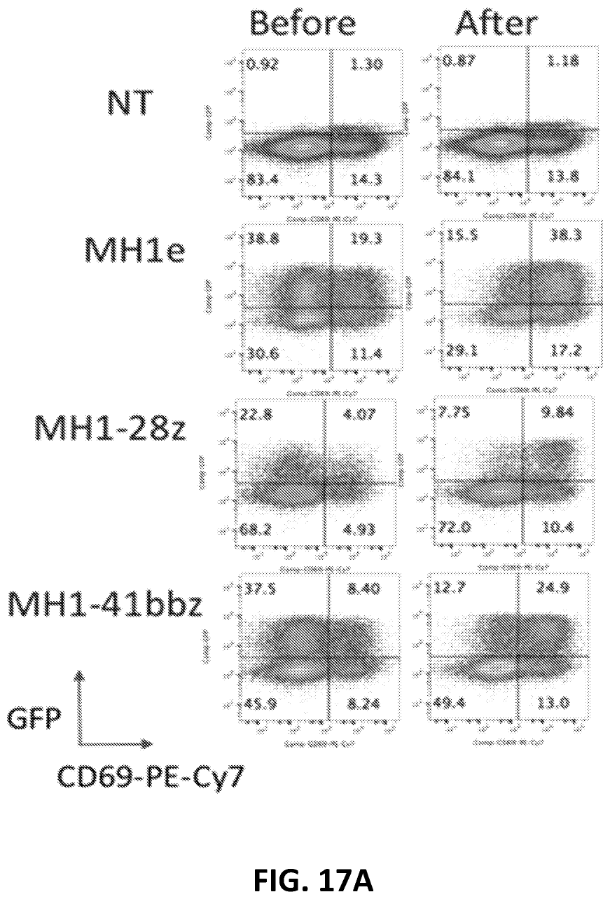

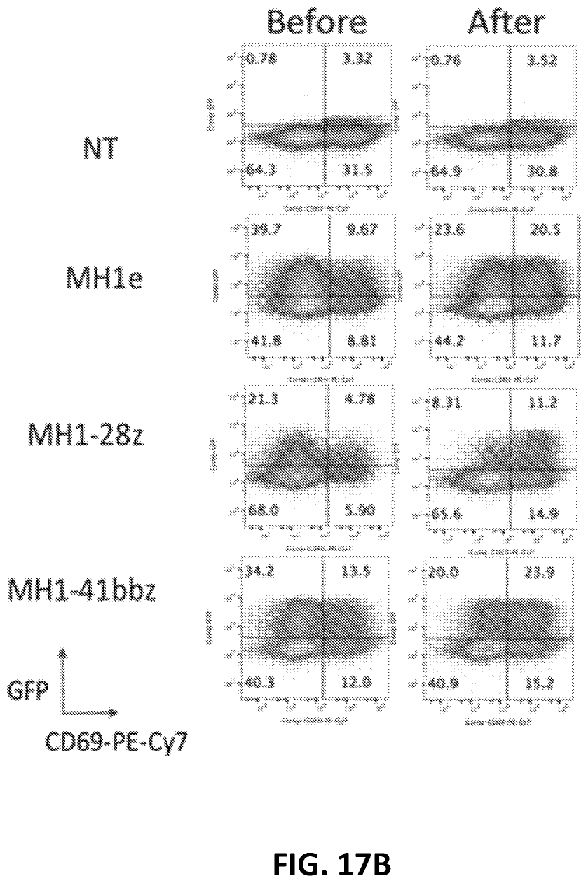

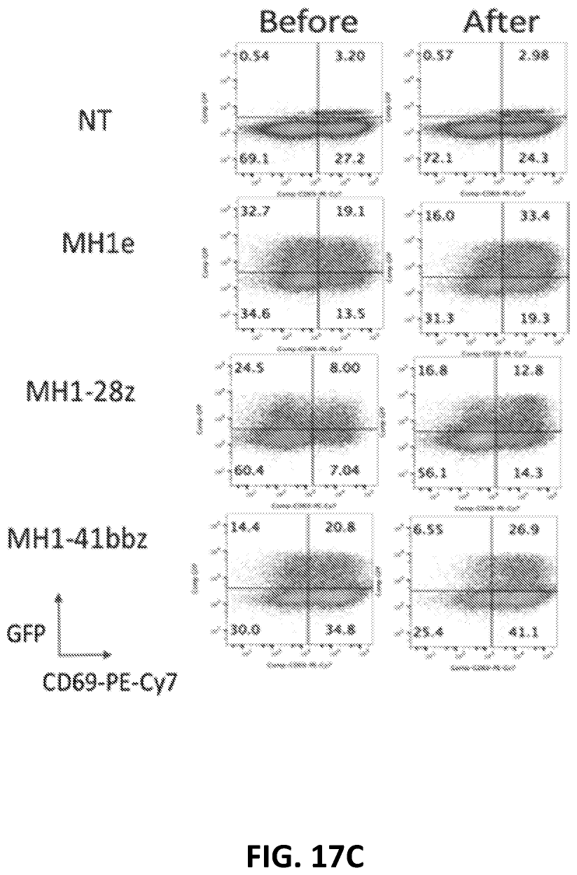

Day 10 post cell culture, TRuC (MH1-CD3ε) and CARs (MH1-CD28ζ and MH1-41BBζ) T cells were assayed for mesothelin scFv expression on T cell surface. TRuC and CARs had similar levels of expression of scFv expression (FIG. 16). Transduced and NT T cells were then placed in 96-well plates coated with mesothelin-Fc and incubated for 24 hours at 37° C. The T cells were then assessed for activation status using CD69 marker (FIGS. 17A-C) and submitted for gene expression analysis.

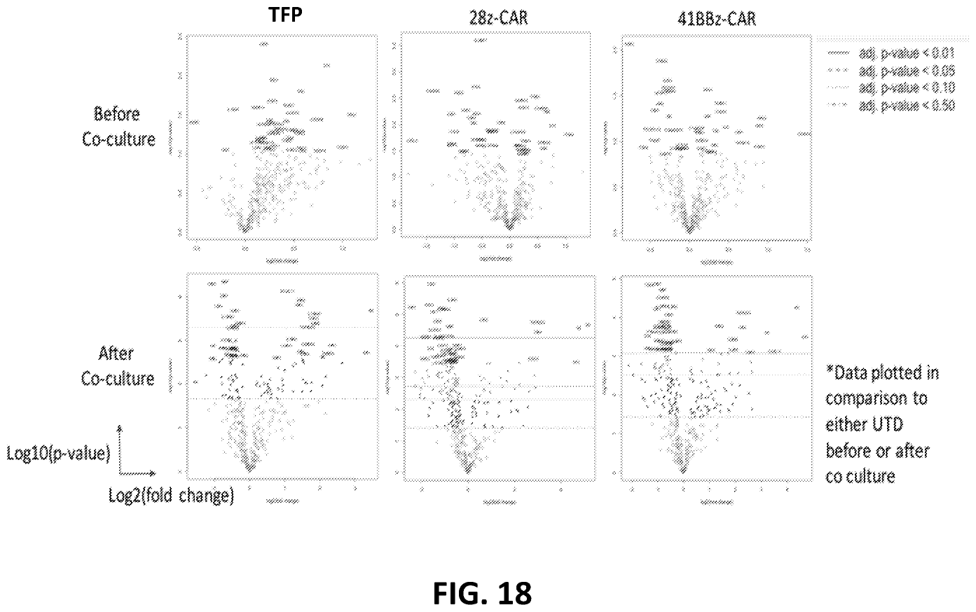

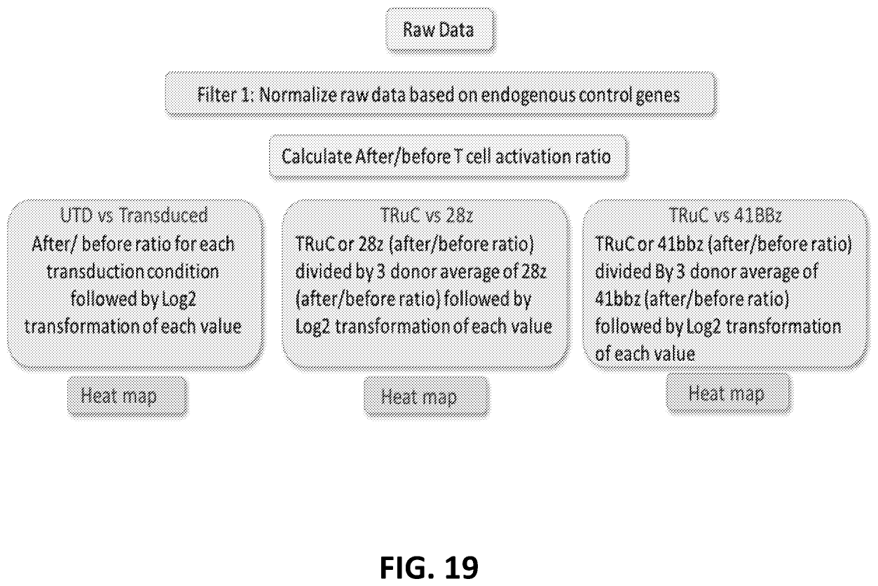

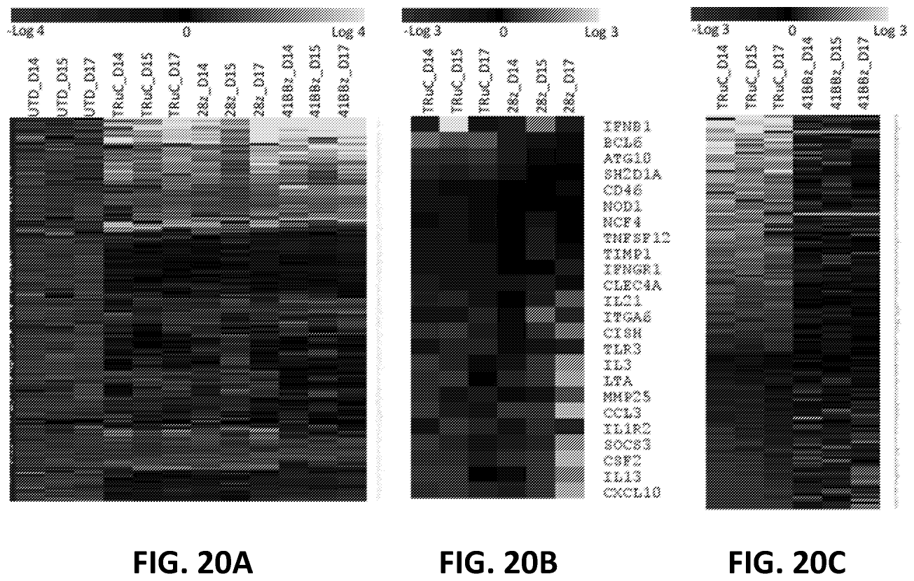

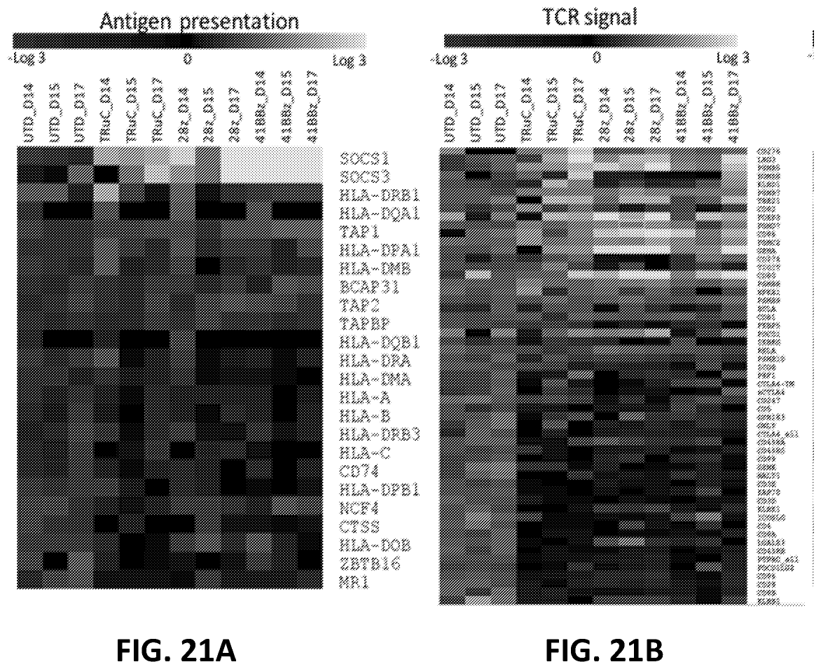

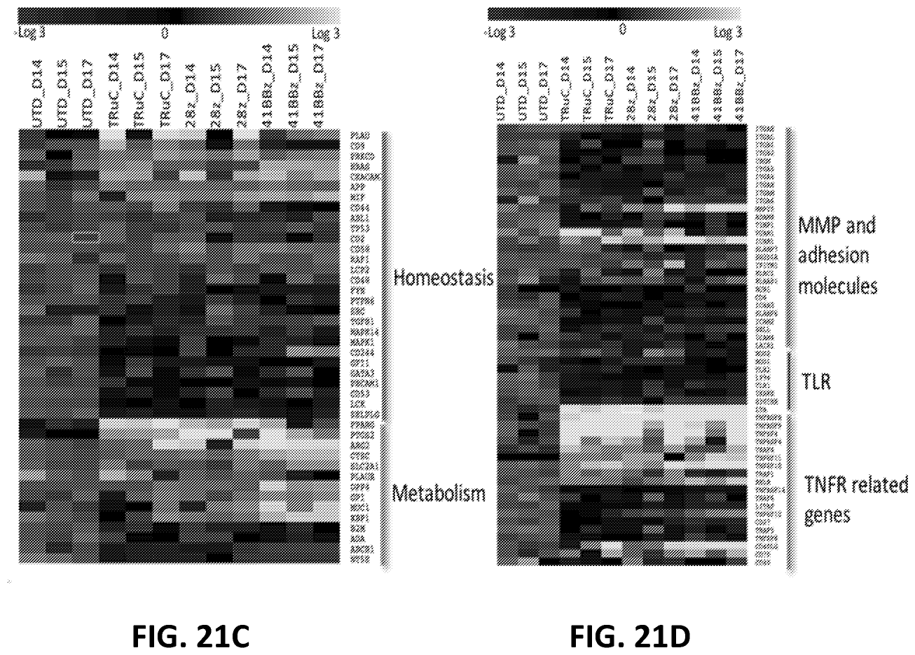

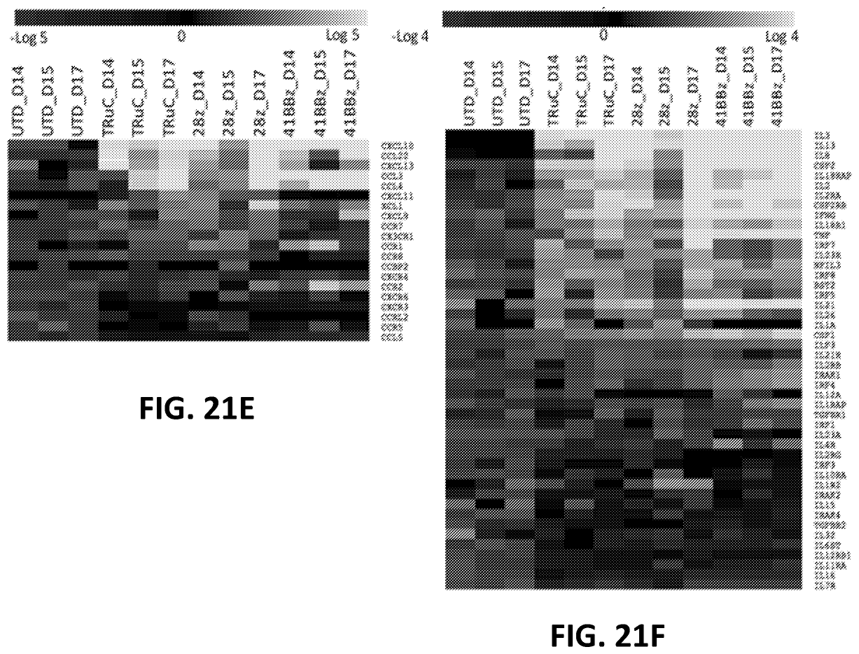

Gene expression analysis revealed a significant change in transduced T cells post-activation compare to NT T cells (FIG. 18). A schematic of gene expression data analysis was shown in FIG. 19. R-program based n-Solver V3 software was used to normalize raw data using endogenous control genes included in the immunology V3 n-String panel. Heat map of the normalized data reveal absence of any significant changes among the transduced cells compared to NT T cells (FIGS. 20A-C). When compared between TRuC and CARs, a significant number of genes were populated (FIGS. 20A-C). Heat map of all transduced genes versus NT genes with genes segregated based on functionality and grouped based on activation profile, reveal that CARs and TRuCs have similar gene regulation profile compared to NT T cells (FIG. 21A-F).

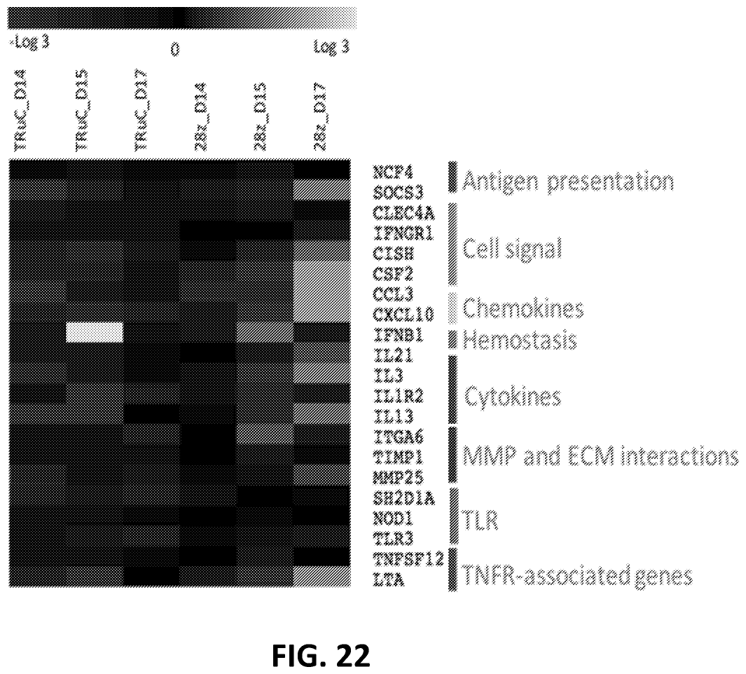

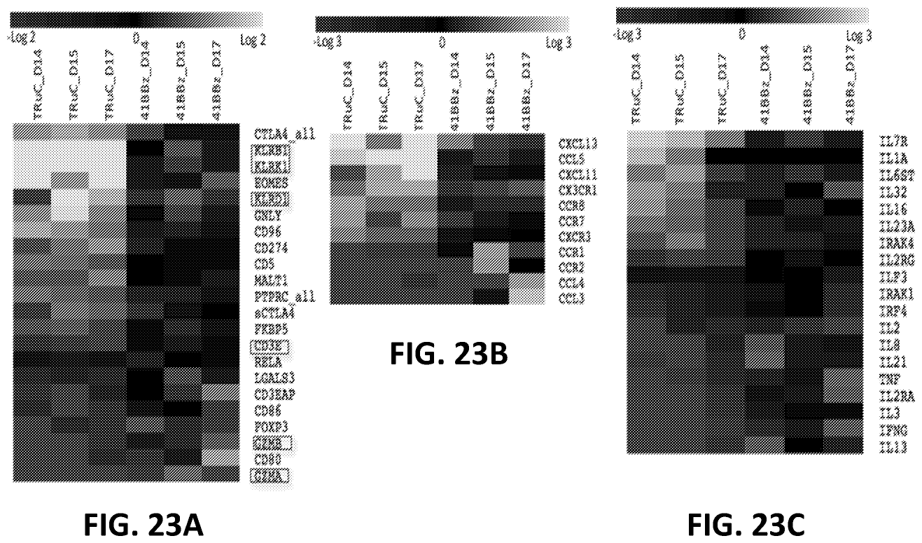

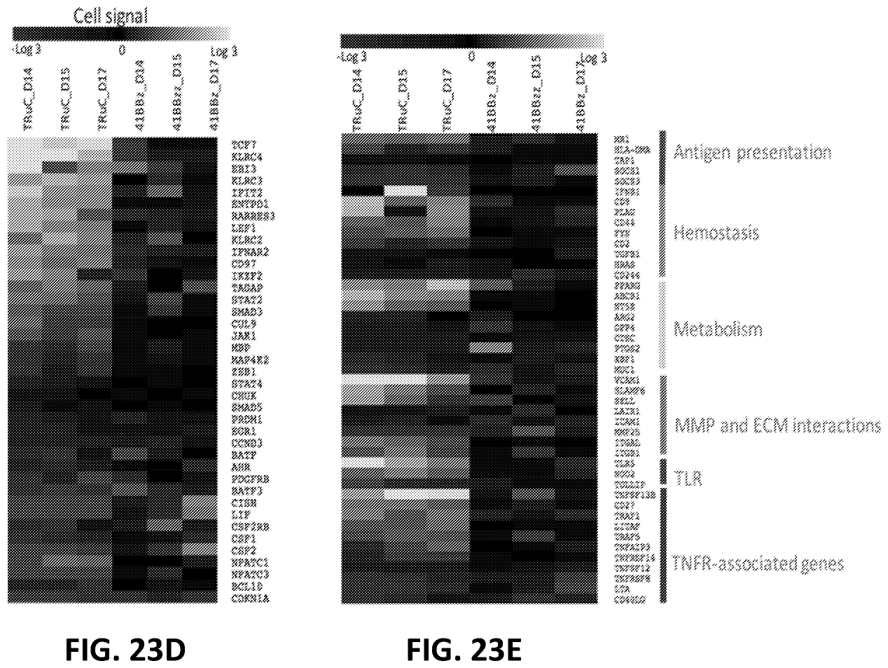

Heatmap of TRuCs vs CD28ζ CAR genes significantly changed or greater than or lower than log 1.5 fold change were segregated based on functionality (FIG. 22). Heatmap revealed that less than 20% of genes analyzed were different compared to TRuCs. While, heat map of all TRuCs vs 41BBζ CAR genes that were significantly changed or greater than or lower than log 1.5 fold change segregated based on functionality, greater than 20% of genes were different from each other (FIGS. 23A-E).

Pathway analysis reveal that most of the genes upregulated for MSLN-specific TRuC T cells includes genes involved in the TCR recruitment and cellular homeostasis, while down regulated genes includes cellular proliferation pathways.

### Example 8. Gene Expression Analysis in CD19-Specific TFP T and CAR-T Cells Upon Antigen Stimulation

### Overview

Assessment of genetic pathway of T cell interaction with antigen-specific tumor cells can reveal the unique property of TRuC T cells in comparison to CAR-T cells. These differences between the gene expression of antigen-stimulated T cells could potentially explain the comparable tumor-specific lysis and persistence of TRuCs in in vivo tumor models and aid in bringing about changes in the platform development to harness TRuC T cell activity in patients.

nCounter analysis (Nanostring), is a powerful digital detection system that uses hybridization-based technology. This tool is capable of multiplexing (more than 500 immunology genes), and profiling all of the individual mRNA gene transcripts directly without the requirement of amplification step. Data generated using this platform is highly accurate in profiling changes in gene expression before and after antigen-stimulation in T cells.

In this study, we reveal the recruitment of TCR components and down regulation of cellular proliferation genes by TRuC T cells when compared to CAR-T cells upon 4 hour tumor cell mediated antigen stimulation.

### Study Design

The study consists of four groups various TRuC and CAR format namely CD3 epsilon (CD3e) TRUC, CD28zeta CAR, 41BBzeta CAR and NT.

### Materials and Methods

Lentivirus Generation

The lentivirus was prepared by transient transfection of 293TN Producer Cell Line (System Biosciences, Inc., LV900A-1) TRUC and CAR constructs were generated using anti-CD19 scfv (clone FMC63) fused to CD3 epsilon chain or CD28-CD3 zeta chain or 41BBz-CD3 zeta chain.

T Cells Isolation and Lentivirus Transduction

CD4 and CD8 T cells were purified from Leukopack sample (Hemacare, Donor 12 Lot #W313716040526, Donor 14 Lot #W313716041176, Donor 15 Lot #W313717041459). Leukaphresis sample was subjected to CD4 and CD8 T cell enrichment using CD4 (Miltenyi, Catalog #130-030-401, Lot #5160914085) and CD8 MACS beads (Miltenyi, Catalog #130-030-801, Lot #5160830314) using automated CliniMACS Prodigy automated system (Miltenyi) according to manufacturer's instructions.

T cells were activated using Dyna beads (Gibco, Catalog #00415447, Lot #1785079) at 1:1 ratio and were maintained in AimV plus AlbuMAX media (Gibco, Catalog #31035-025) in 5% hAB serum (Gemini Products, Catalog #100-318, lot #H605oo1), and 1% antibiotics (Gibco, Catalog #15240-062, Lot #1734036) in presence of 300 IU/ml IL-2 (Peprotech, Catalog #200-02, lot #051512). Dynabead activated T cells were transduced with lentivirus at 1MOI respectively in presence of polybrene (5ug/ml) (Millipore, Catalog #TR-1003-G) and spinoculation at 100G for 100 minutes. A total of two rounds of transductions were performed at 24 hour and 48-hour post transduction.

Transduction Efficiency and Activation Determination:

Transduction efficiency was determined by flow cytometry. T cells were stained using anti-CD3 APC (Clone, UCHT1. BD Biosciences Catalog #340440, Lot #6005787), anti-CD4-Pacific blue (CloneRPAT4, Biolegend, Catalog #300521, Lot #B231611), anti-CD8-APCCY7 (Clone SK1, BD Biosciences, Catalog #557834, Lot #, 6082865), Goat anti mouse FAB (Invitrogen), CD69-AF 700 (Clone FN50, Catalog #560739, Lot #7051802) and isotype controls, APC Mouse IgG1, k Isotype Control (Clone X40, BD Biosciences, Catalog #340442) Pacific Blue isotype control (Clone MOPC-21, BD Biosciences, Catalog #558120), APCCY7 IgG1 isotype control (BD Biosciences, Catalog #557873), AF700 IgG1 isotype control (Clone 27-35, BD Biosciences, Catalog #560543). Cells were analyzed using BD-LSRII Fortessa X20.

T Cell Co-Culture and Purification:

2×10⁶ TRuC or CAR-T cells were co-cultured with 2×10⁶ Raji cells (1:1::E:T ratio) for 4 hrs. Post co-culture the co-culture mixture was tagged to CD19 beads (Miltenyi, Catalog number #130-050-301, Lot #5160318239) for 30 mins at 4° C. The tagged mixture of cells was passed through magnetic column to remove tumor cells and elute out T cells.

Bioinformatics Data Analysis:

The raw count for each transduction condition was normalized using the endogenous control (up to 15 genes used). Ratio of genes upregulated or down regulated after co-culture and before co-culture was calculated for each transduction condition. Statistical t-test was used to calculate p-value of ratio for average of each donor in TRuC and CARs. R-program based nSolver V3 was used to score genes that are at least log 1.5 upregulated or down regulated specifically in TRuCs compared to CARs. Heatmaps were generated using the nSolver V3 software.

Pathway analysis was performed by entering the significant upregulated or down regulated TRuC genes list into www.reactome.org software and genes based on FDR hits for each pathway was plotted in a graphical representation.

### Results

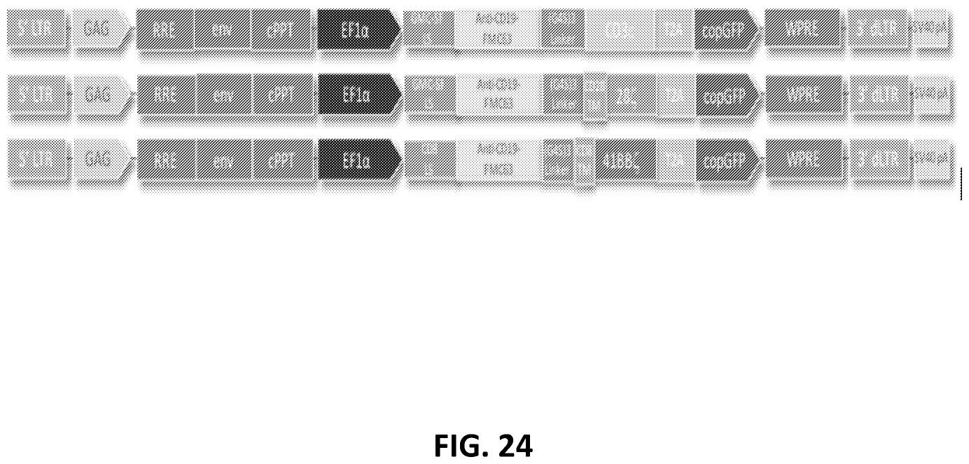

T cells were enriched for CD4 and CD8 T cells and transduced with indicated lentivirus vectors (FIG. 24). Lentivirus vector for each transgene contained a T2A cleavage site which allowed us to evaluate transduction efficiency and surface expression of these novel transgenes.

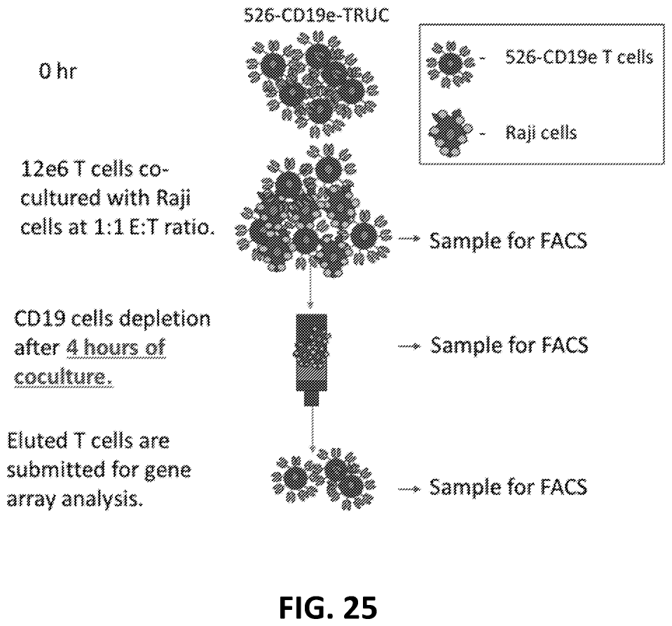

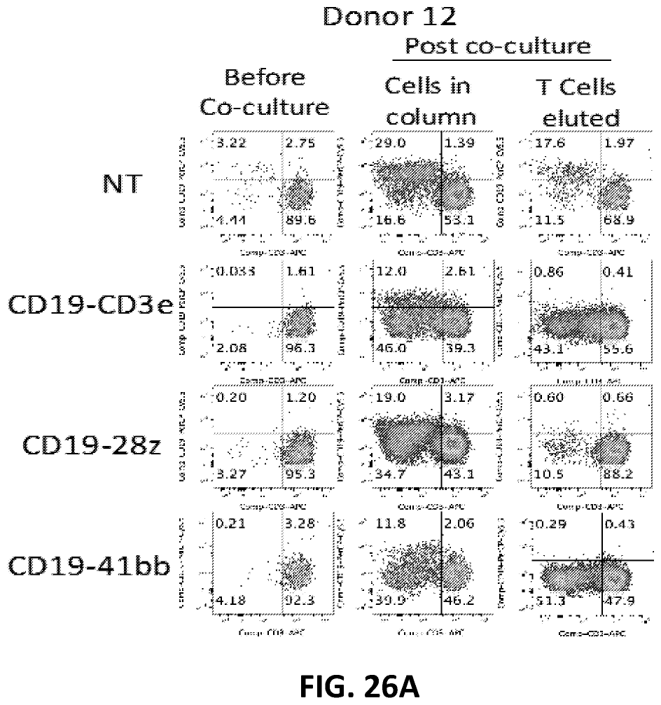

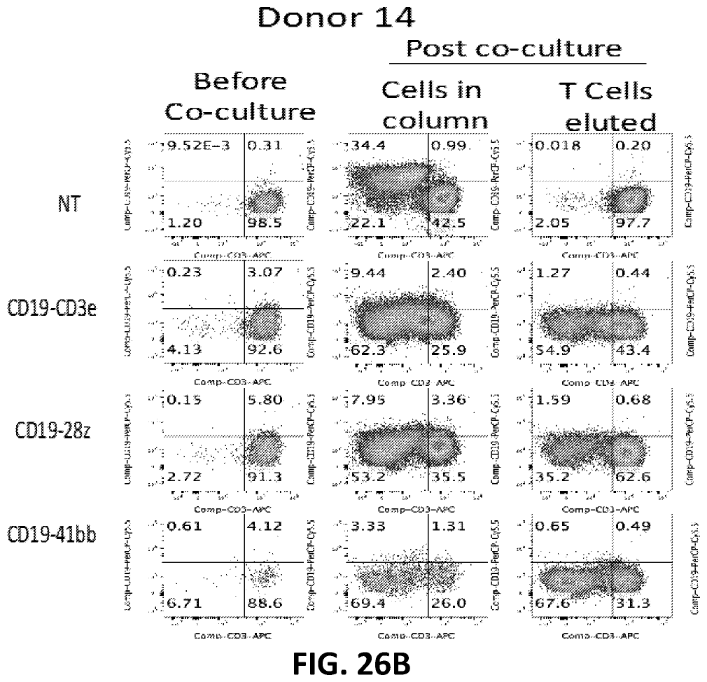

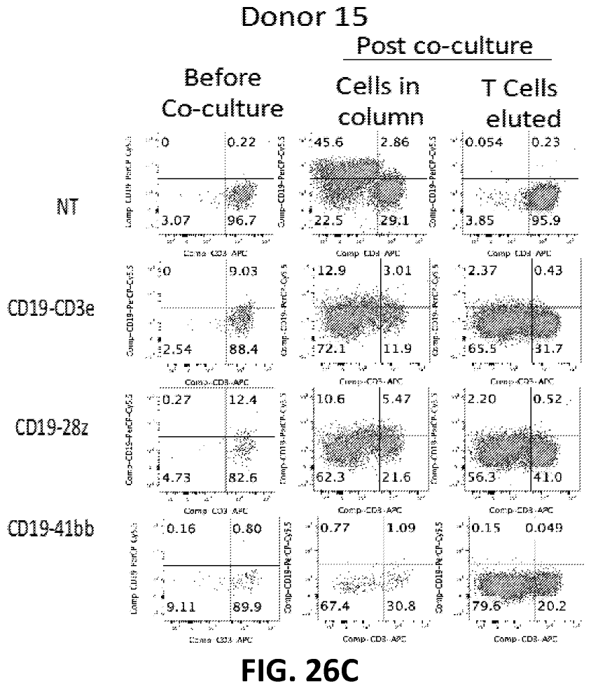

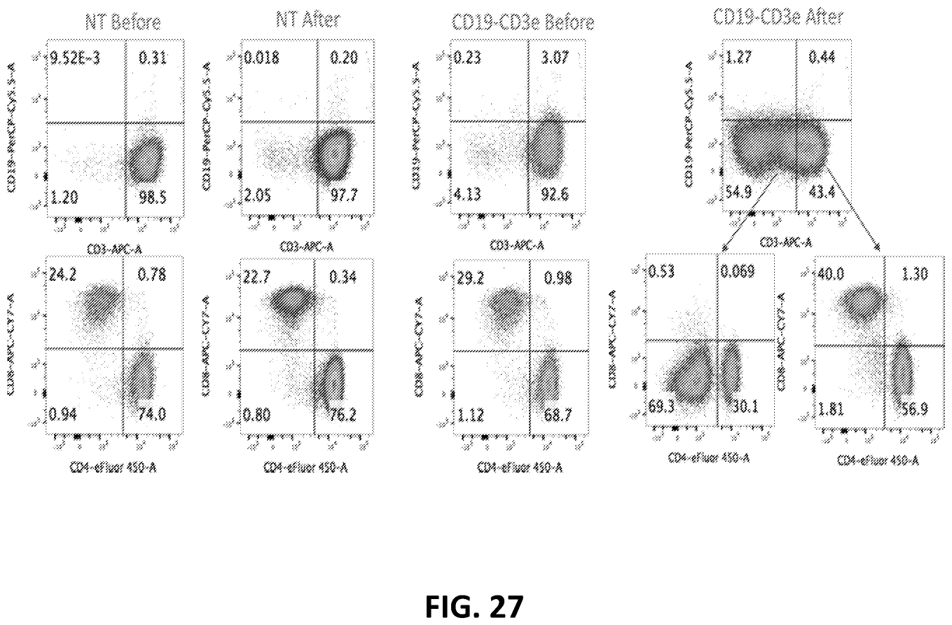

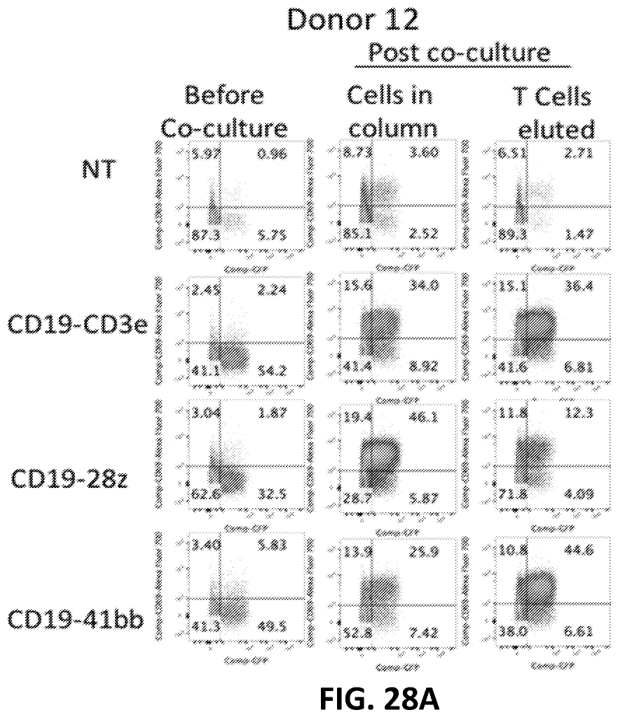

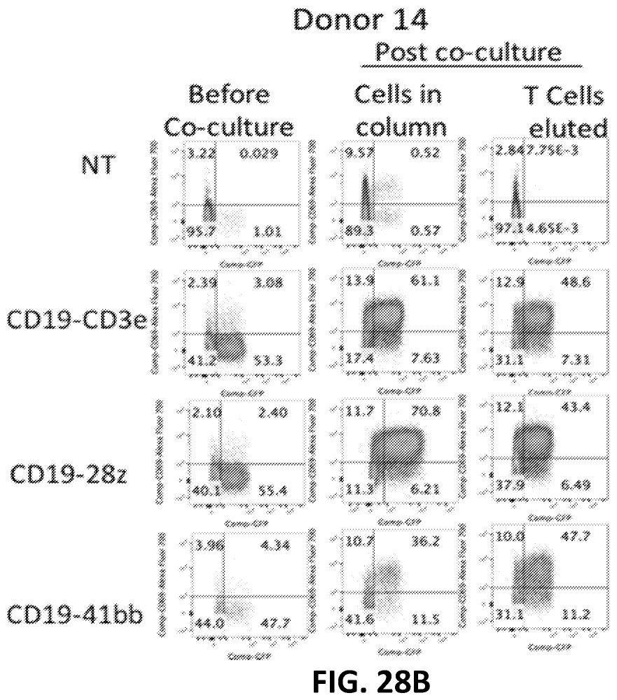

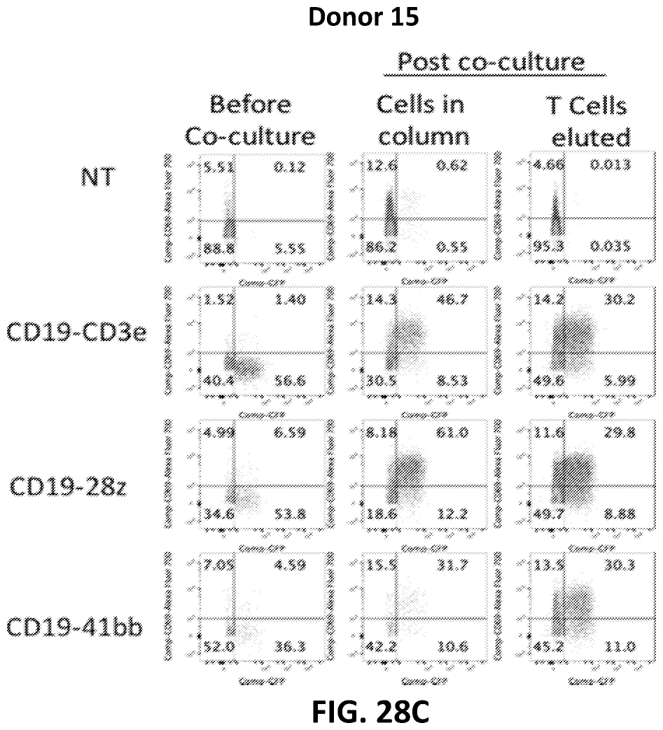

A schematic of experimental plan is shown on FIG. 25. Day 7 post cell culture, TRuC (CD19-CD3e) and CARs (CD19-CD28z and CD19-41BBz) T cells were co-cultured with Raji cells in a 1:1 E:T ratio for 4 hours. Post co-culture, the tumor and T cell mixture was tagged with CD19 magnetic beads and passed through a magnetic column to remove tumor cells. Our data revealed 4 hour co-culture created a strong attachment between the T cells and tumors cells (except in NT condition) and magnetic sorting could yield a pure population of T cells (FIGS. 26A-C and FIG. 27). These T cells did maintain the TRuC and CAR expression (represented by the GFP levels) and were substantially activated (represented by CD69-AF-700 levels) post co-culture (FIGS. 28A-C).

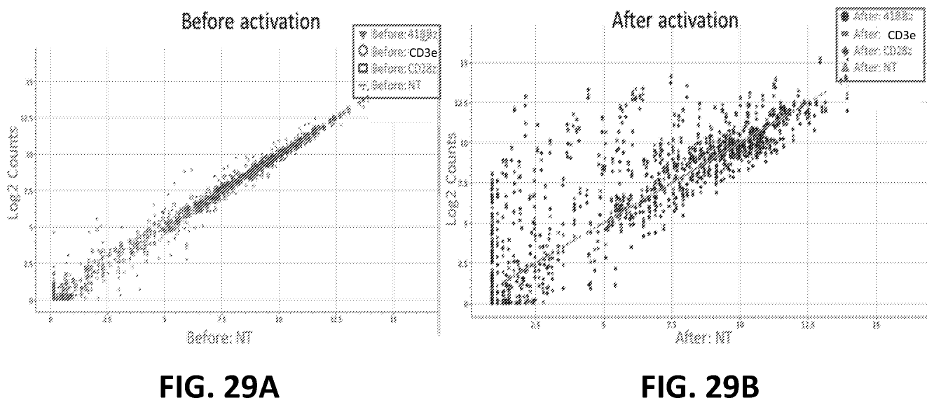

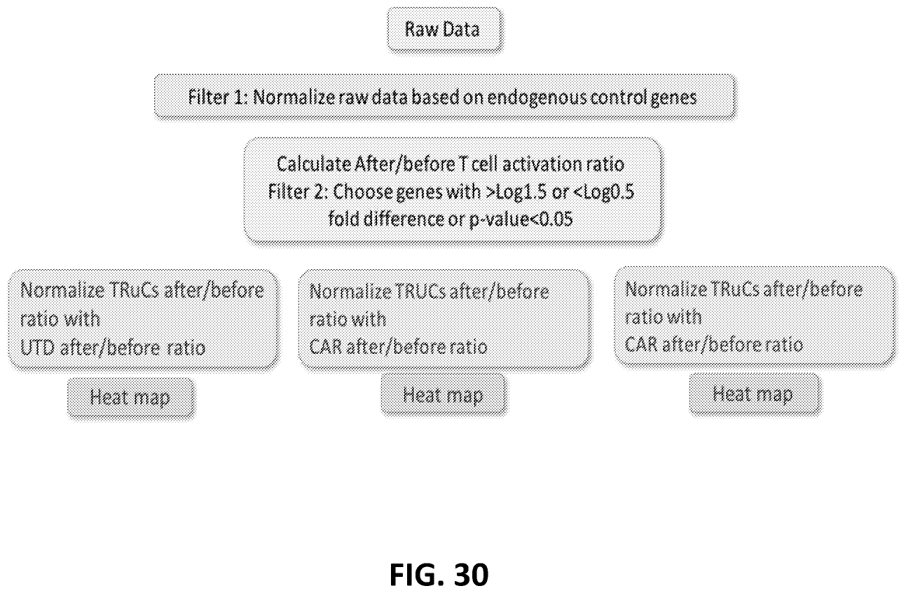

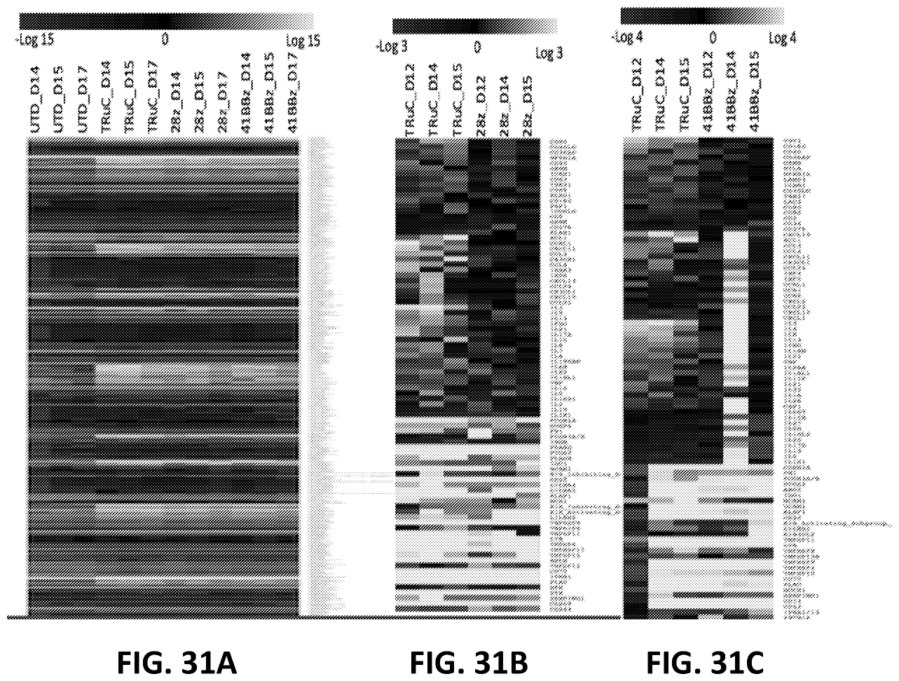

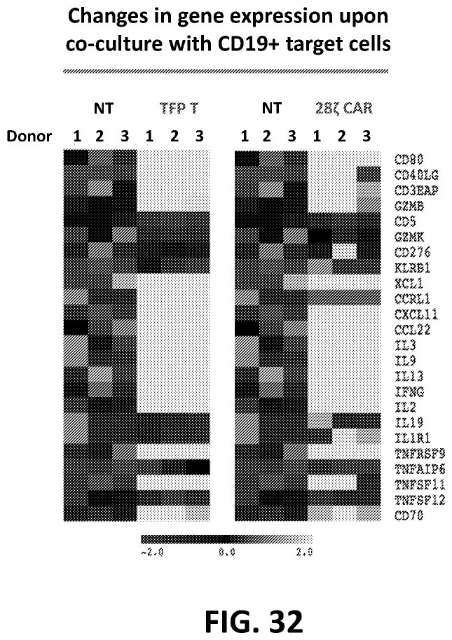

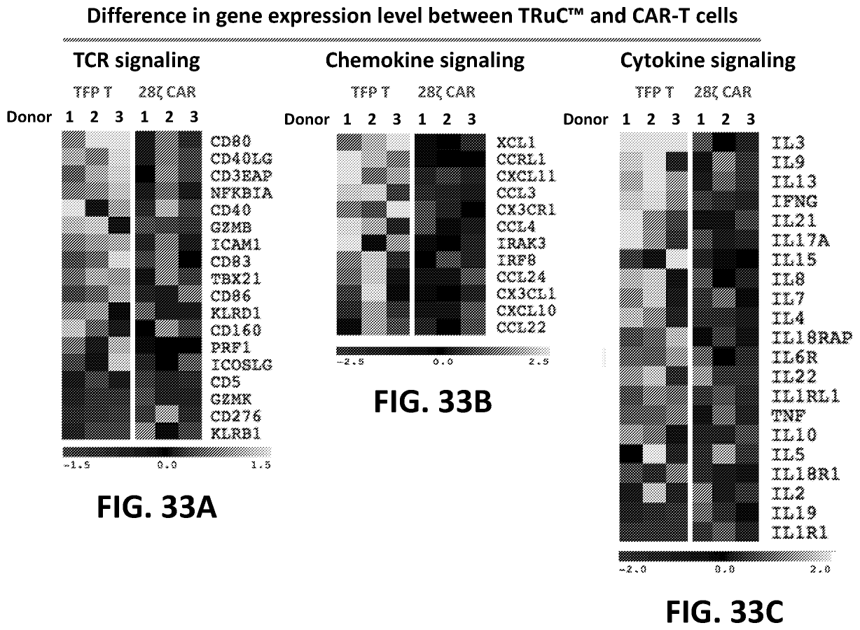

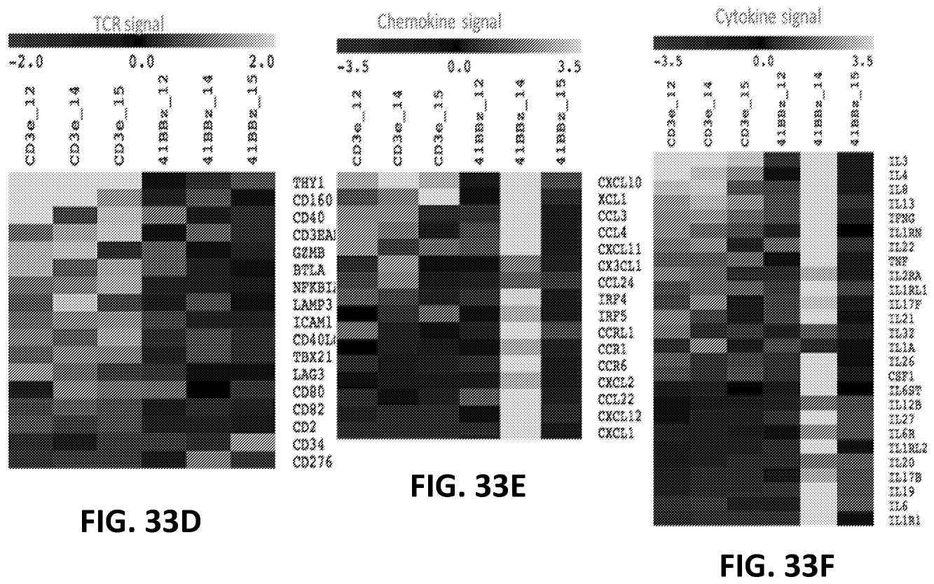

Gene expression analysis revealed the activation and down regulation of several genes (Log 2 fold) post co-culture (FIGS. 29A and B). A schematic of gene expression data analysis was shown in FIG. 30. The gene raw counts were first normalized to endogenous control, followed by creation of ratio of after stimulation with tumor cell divided by before stimulation. This ratio for transduced condition was later normalized to ratio of NT cells. R-program based n-Solver V3 software was used to normalize raw data using endogenous control genes included in the immunology V3 n-String panel. Heat map of the normalized data in comparison to normalized NT data reveal absence of any significant changes among the transduced cells compared to NT cells (FIGS. 31A-C). When gene array for TRuC transduced T cells was compared to CARs using after co-culture divided by before co-culture ratio greater than 50 genes were significantly upregulated in TRuCs compared to 41BBz or 28z CARs (FIGS. 32 and 33A-F). These genes were then categorized based on T cell functionality. Any gene that might have influence in other immune cell phenotype or not significant or have greater than 0.5 fold or less than 1.5 fold change were excluded. TRuC-transduced genes that are either significantly upregulated or down regulated was plotted on a heat map to reveal the level of difference in regulation (FIGS. 32 and 33A-F).

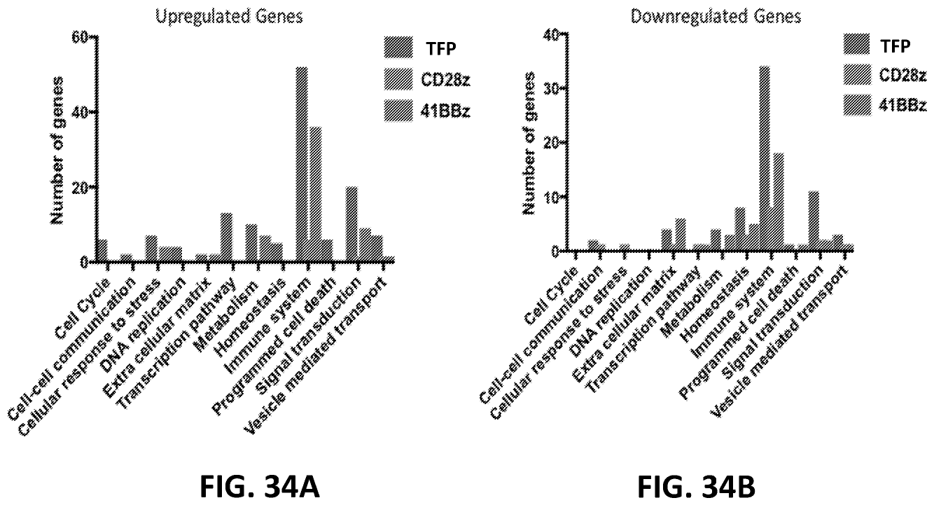

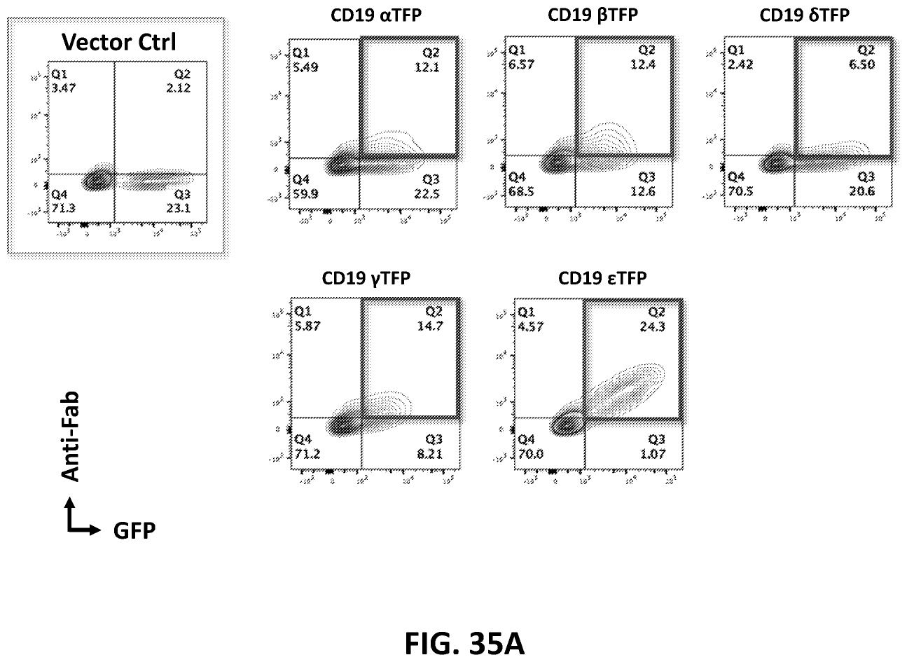

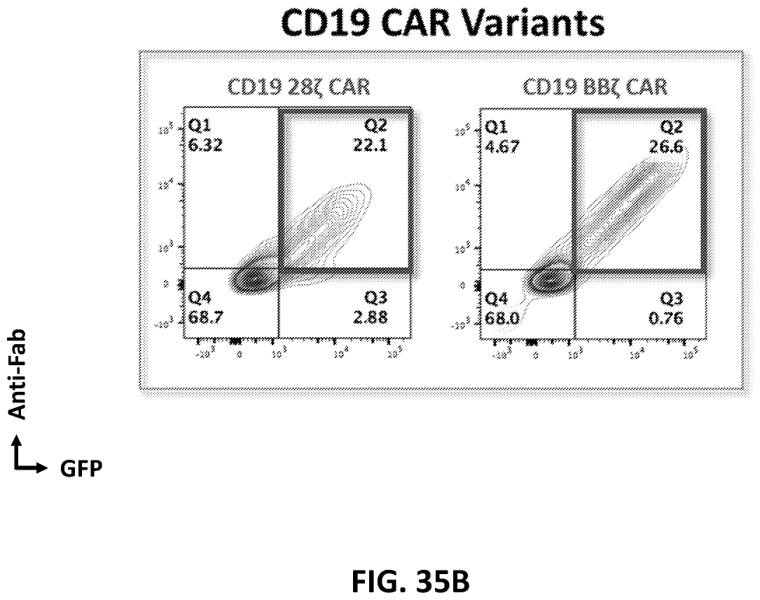

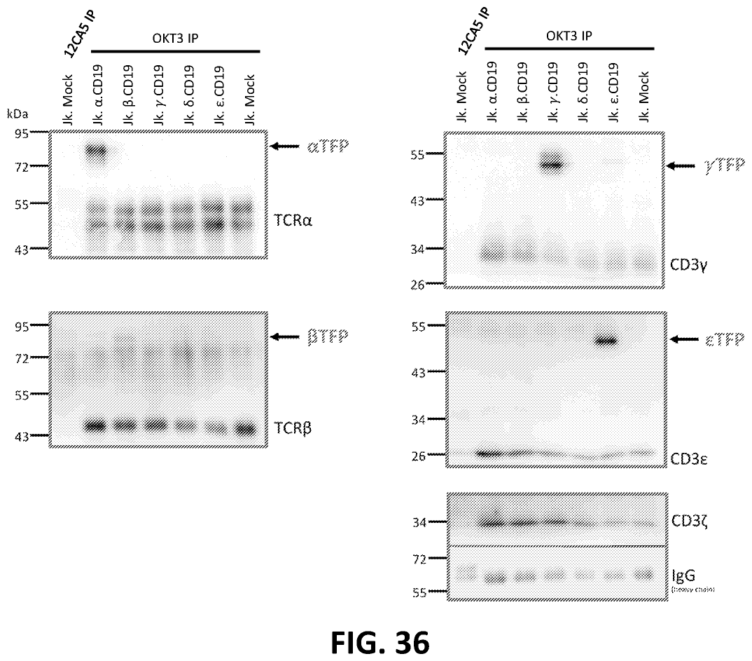

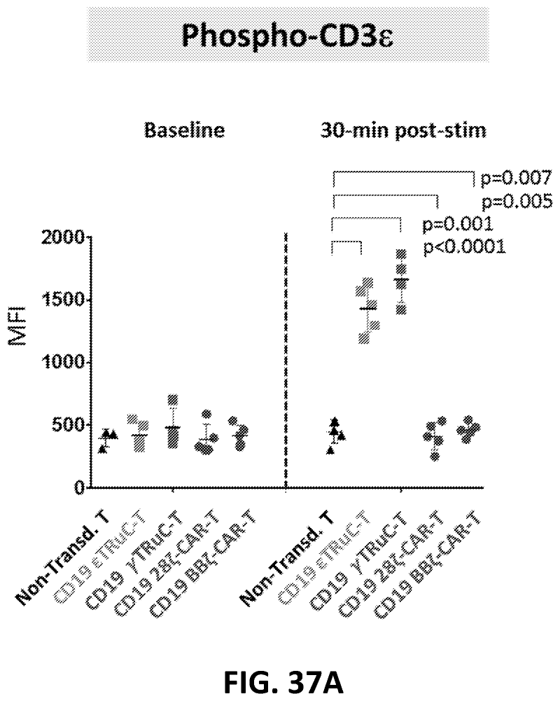

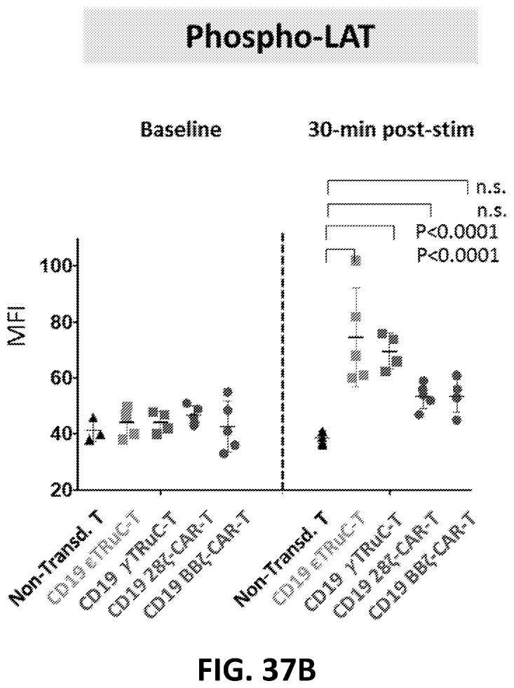

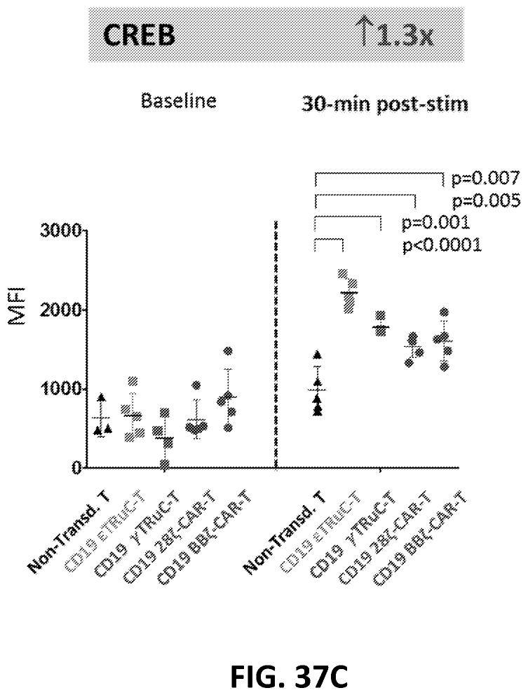

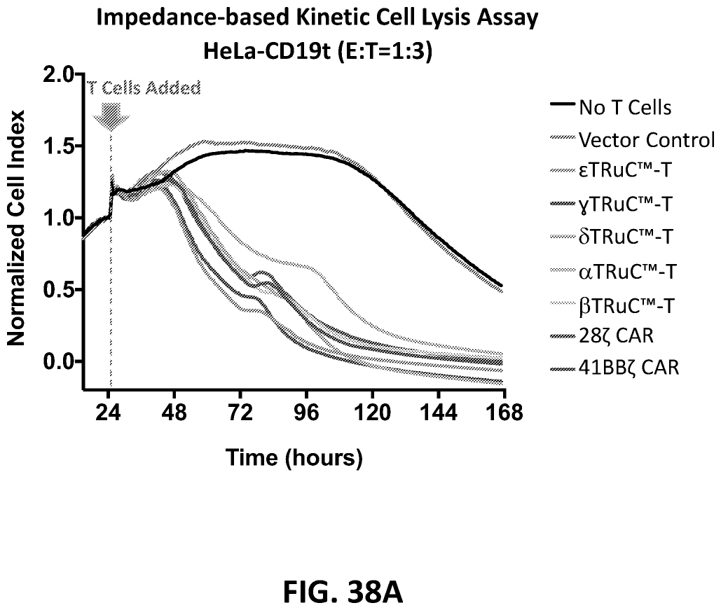

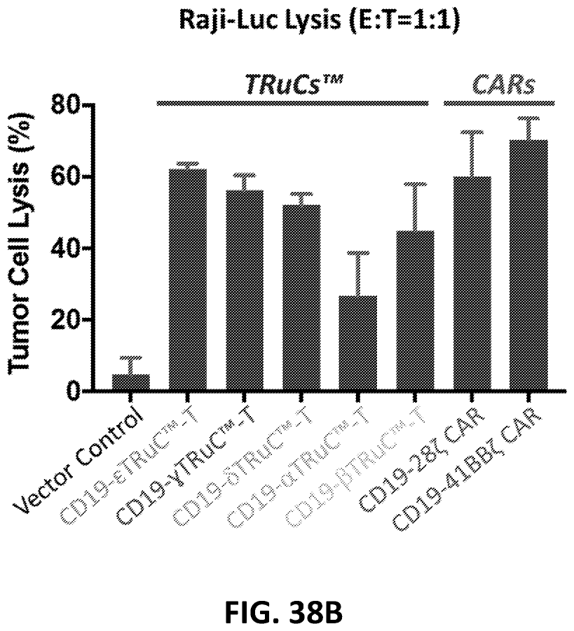

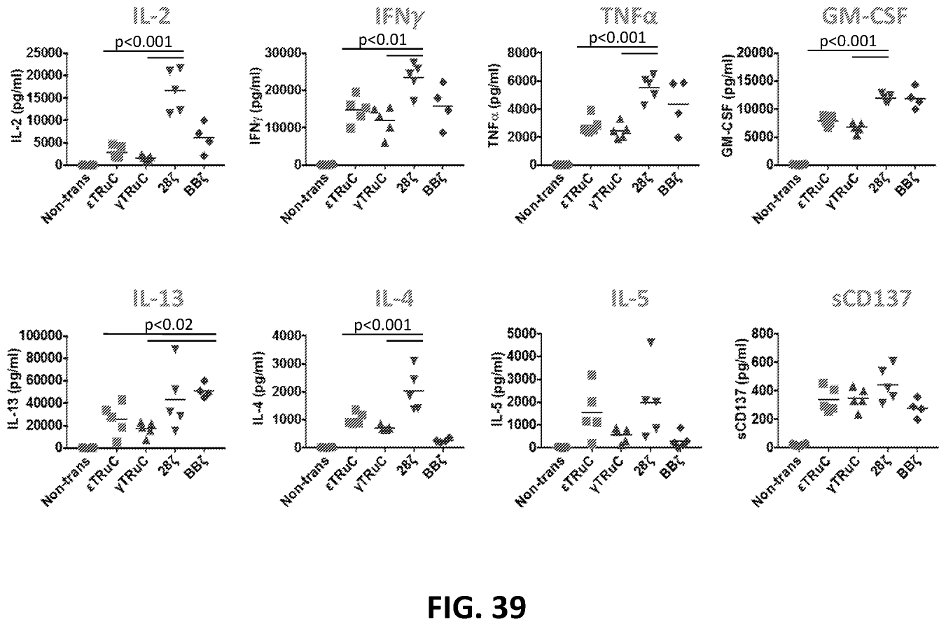

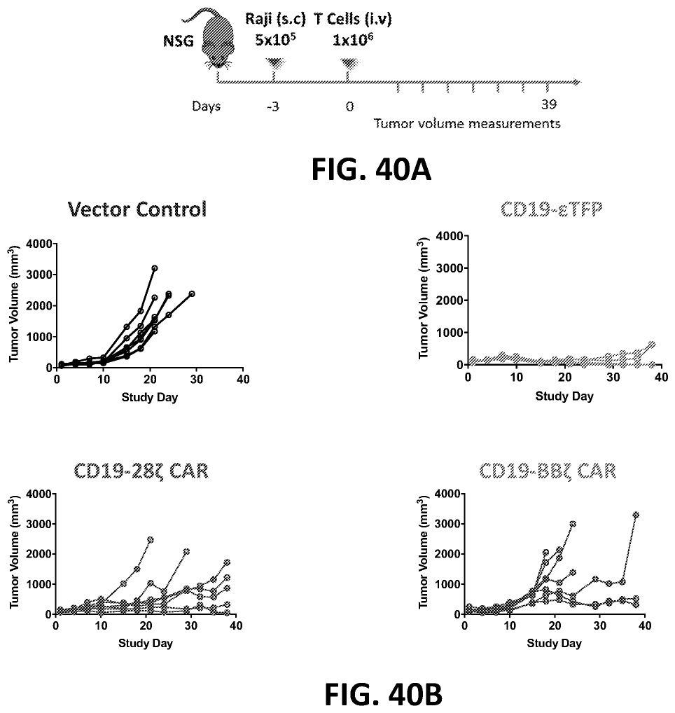

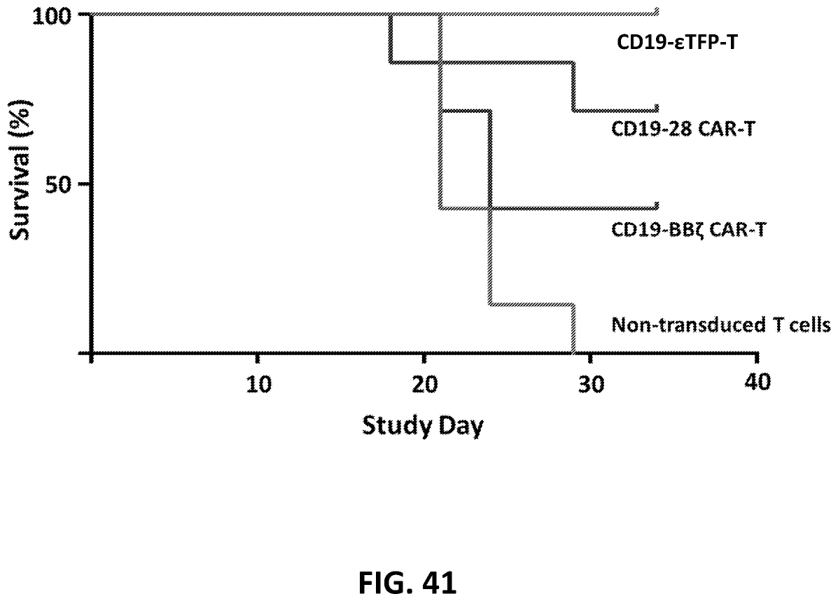

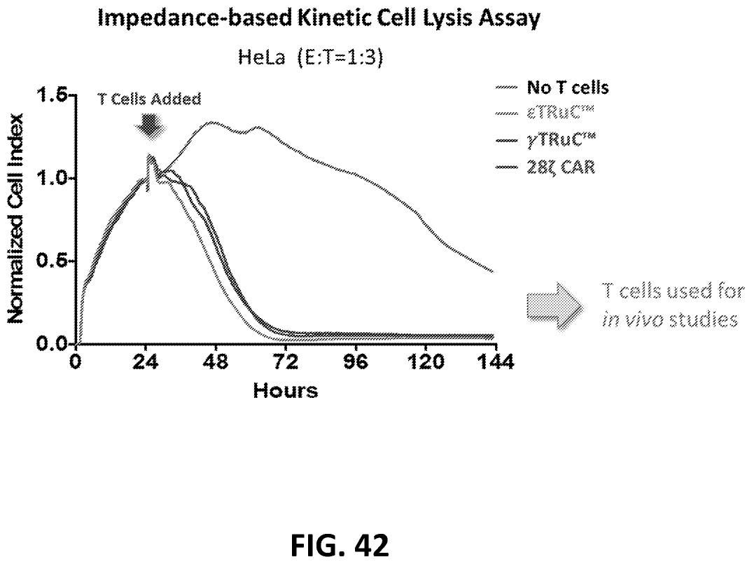

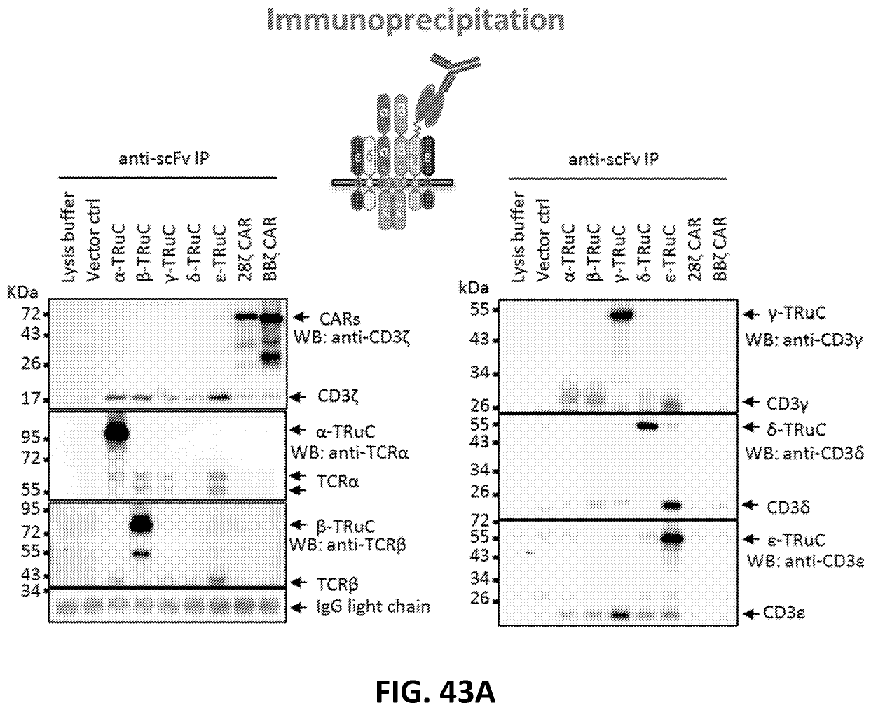

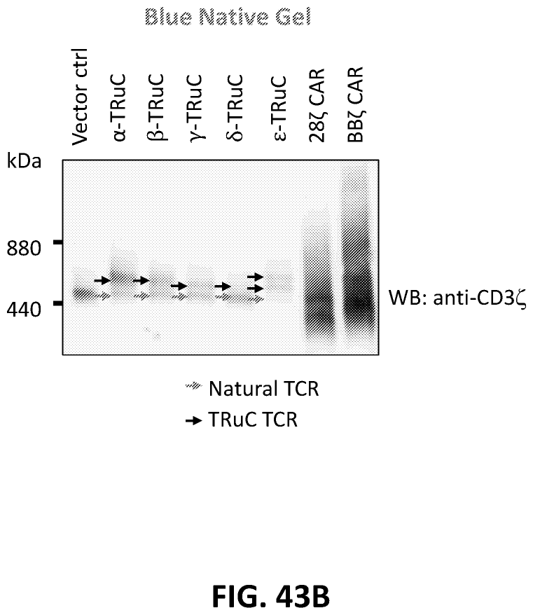

Pathway analysis reveal that most of the genes upregulated for CD19 TRuC T cells includes genes involved in the TCR recruitment, including genes involved in Fc epsilon receptor signaling, with increased in IL1, IL4 and IL13 regulatory cytokine gene expression, increase in genes involved in TNF-receptor family and cellular homeostasis, while down regulated genes includes cellular proliferation pathways (FIGS. 34A and B).

### ENDNOTES

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

SEQUENCES:

| SEQ | | |
|---|---|---|
| ID | | |
| NO. | Name | Sequence |
| 1 | Short | GGGGSGGGGSGGGGSLE |
| | Linker 1 | |
| 2 | Short | AAAGGGGSGGGGSGGGGSLE |
| | Linker 2 | |
| 3 | Long | AAAIEVMYPPPYLGGGGSGGGGSGGGGSLE |
| | Linker | |
| 4 | human | MQSGTHWRVLGLCLLSVGVWGQDGNEEMGGITQTPYKVSISG |
| | CD3-ϵ | TTVILTCPQYPGSEILWQHNDKNIGGDEDDKNIGSDEDHLSLKE |
| | | FSELEQSGYYVCYPRGSKPEDANFYLYLRARVCENCMEMDVM |
| | | SVATIVIVDICITGGLLLLVYYWSKNRKAKAKPVTRGAGAGGR |
| | | QRGQNKERPPPVPNPDYEPIRKGQRDLYSGLNQRRI |
| 5 | human | MEQGKGLAVLILAIILLQGTLAQSIKGNHLVKVYDYQEDGSVL |
| | CD3-γ | LTCDAEAKNITWFKDGKMIGFLTEDKKKWNLGSNAKDPRGMY |
| | | QCKGSQNKSKPLQVYYRMCQNCIELNAATISGFLFAEIVSIFVL |
| | | AVGVYFIAGQDGVRQSRASDKQTLLPNDQLYQPLKDREDDQY |
| | | SHLQGNQLRRN |
| 6 | human | MEHSTFLSGLVLATLLSQVSPFKIPIEELEDRVFVNCNTSITWVE |
| | CD3-δ | GTVGTLLSDITRLDLGKRILDPRGIYRCNGTDIYKDKESTVQVH |
| | | YRMCQSCVELDPATVAGIIVTDVIATLLLALGVFCFAGHETGRL |
| | | SGAADTQALLRNDQVYQPLRDRDDAQYSHLGGNWARNKS |
| 7 | human | MKWKALFTAAILQAQLPITEAQSFGLLDPKLCYLLDGILFIYGVI |
| | CD3-ζ | LTALFLRVKFSRSADAPAYQQGQNQLYNELNLGRREEYDVLD |
| | | KRRGRDPEMGGKPQRRKNPQEGLYNELQKDKMAEAYSEIGM |
| | | KGERRRGKGHDGLYQGLSTATKDTYDALHMQALPPR |
| 8 | human | MAGTWLLLLLALGCPALPTGVGGTPFPSLAPPIMLLVDGKQQM |
| | TCR α- | VVVCLVLDVAPPGLDSPIWFSAGNGSALDAFTYGPSPATDGTW |
| | chain | TNLAHLSLPSEELASWEPLVCHTGPGAEGHSRSTQPMHLSGEAS |
| | | TARTCPQEPLRGTPGGALWLGVLRLLLFKLLLFDLLLTCSCLCD |
| | | PAGPLPSPATTTRLRALGSHRLHPATETGGREATSSPRPQPRDR |
| | | RWGDTPPGRKPGSPVWGEGSYLSSYPTCPAQAWCSRSALRAPS |
| | | SSLGAFFAGDLPPPLQAGA |
| 9 | human | PNIQNPDPAVYQLRDSKSSDKSVCLFTDFDSQTNVSQSKDSDV |
| | TCR α- | YITDKTVLDMRSMDFKSNSAVAWSNKSDFACANAFNNSIIPED |
| | chain C | TFFPSPESSCDVKLVEKSFETDTNLNFQNLSVIGFRILLLKVAGF |
| | region | NLLMTLRLWSS |
| 10 | human | MAMLLGASVLILWLQPDWVNSQQKNDDQQVKQNSPSLSVQE |
| | TCR α- | GRISILNCDYTNSMFDYFLWYKKYPAEGPTFLISISSIKDKNEDG |
| | chain V | RFTVFLNKSAKHLSLHIVPSQPGDSAVYFCAAKGAGTASKLTFG |
| | region | TGTRLQVTL |
| | CTL-L17 | |
| 11 | human | EDLNKVFPPEVAVFEPSEAEISHTQKATLVCLATGFFPDHVELS |
| | TCR β- | WWVNGKEVHSGVSTDPQPLKEQPALNDSRYCLSSRLRVSATF |
| | chain C | WQNPRNHFRCQVQFYGLSENDEWTQDRAKPVTQIVSAEAWGR |
| | region | ADCGFTSVSYQQGVLSATILYEILLGKATLYAVLVSALVLMAM |
| | | VKRKDF |
| 12 | human | MGTSLLCWMALCLLGADHADTGVSQNPRHNITKRGQNVTFRC |
| | TCR β- | DPISEHNRLYWYRQTLGQGPEFLTYFQNEAQLEKSRLLSDRFSA |
| | chain V | ERPKGSFSTLEIQRTEQGDSAMYLCASSLAGLNQPQHFGDGTRL |
| | region | SIL |
| | CTL-L17 | |
| 13 | human | MDSWTFCCVSLCILVAKHTDAGVIQSPRHEVTEMGQEVTLRCK |
| | TCR β- | PISGHNSLFWYRQTMMRGLELLIYFNNNVPIDDSGMPEDRFSA |
| | chain V | KMPNASFSTLKIQPSEPRDSAVYFCASSFSTCSANYGYTFGSGT |
| | region | RLTVV |
| | YT35 | |
| 14 | MSLN | acgcgtgtagtcttatgcaatactcttgtagtcttgcaacatggtaacgatgagttagcaacatgcct |
| | DNA | tacaaggagagaaaaagcaccgtgcatgccgattggtggaagtaaggtggtacgatcgtgccttatta |
| | Seq. | ggaaggcaacagacgggtctgacatggattggacgaaccactgaattgccgcattgcagagatattgt |
| | | atttaagtgcctagctcgatacaataaacgggtctctctggttagaccagatctgagcctgggagctc |
| | | tctggctaactagggaacccactgcttaagcctcaataaagcttgccttgagtgcttcaagtagtgtg |
| | | tgcccgtctgttgtgtgactctggtaactagagatccctcagacccttttagtcagtgtggaaaatct |
| | | ctagcagtggcgcccgaacagggacctgaaagcgaaagggaaaccagagctctctcgacgcaggactc |
| | | ggcttgctgaagcgcgcacggcaagaggcgaggggcggcgactggtgagtacgccaaaaattttg |
| | | actagcggaggctagaaggagagagatgggtgcgagagcgtcagtattaagcgggggagaatta |
| | | gatcgcgatgggaaaaaattcggttaaggccagggggaaagaaaaaatataaattaaaacatatagt |
| | | atgggcaagcagggagctagaacgattcgcagttaatcctggcctgttagaaacatcagaaggctgt |
| | | agacaaatactgggacagctacaaccatcccttcagacaggatcagaagaacttagatcattatataa |
| | | tacagtagcaaccctctattgtgtgcatcaaaggatagagataaaagacaccaaggaagctttagaca |
| | | agatagaggaagagcaaaacaaaagtaagaccaccgcacagcaagcggccactgatcttcagac |
| | | ctggaggaggagatatgagggacaattggagaagtgaattatataaatataaagtagtaaaaattgaa |
| | | ccattaggagtagcacccaccaaggcaaagagaagagtggtgcagagagaaaaaagagcagtgg |
| | | gaataggagctttgttccttgggttcttgggagcagcaggaagcactatgggcgcagcctcaatgac |
| | | gctgacggtacaggccagacaattattgtctggtatagtgcagcagcagaacaatttgctgagggcta |
| | | ttgaggcgcaacagcatctgttgcaactcacagtctggggcatcaagcagctccaggcaagaatcct |
| | | ggctgtggaaagatacctaaaggatcaacagctcctggggatttggggttgctctggaaaactcattt |
| | | gcaccactgctgtgccttggaatgctagttggagtaataaatctctggaacagattggaatcacacga |
| | | cctggatggagtgggacagagaaattaacaattacacaagcttaatacactccttaattgaagaatcg |
| | | caaaaccagcaagaaaagaatgaacaagaattattggaattagataaatgggcaagtttgtggaattg |
| | | gtttaacataacaaattggctgtggtatataaaattattcataatgatagtaggaggcttggtaggtt |
| | | taagaatagtttttgctgtactttctatagtgaatagagttaggcagggatattcaccattatcgttt |
| | | cagacccacctcccaaccccgaggggacccgacaggcccgaaggaatagaagaagaaggtggagagag |
| | | agacagagacagatccattcgattagtgaacggatctcgacggtatcggttaacttttaaaagaaaag |
| | | gggggattggggggtacagtgcaggggaaagaatagtagacataatagcaacagacatacaaacta |
| | | aagaattacaaaaacaaattacaaaattcaaaattttatcgatactagtattatgcccagtacatgac |
| | | cttatgggactttcctacttggcagtacatctacgtattagtcatcgctattaccatggtgatgcggt |
| | | tttggcagtacatcaatgggcgtggatagcggtttgactcacggggatttccaagtctccaccccatt |
| | | gacgtcaatggga |
| | | gtttgttttggcaccaaaatcaacgggactttccaaaatgtcgtaacaactccgccccattgacgcaa |
| | | atgggcggtaggcgtgtacggtgggaggtttatataagcagagctcgtttagtgaaccgtcagatcgc |
| | | ctggagacgccatccacgctgttttgacctccatagaagattctagagccgccaccatgcttctcctg |
| | | gtgacaagccttctgctctgtgagttaccacacccagcattcctcctgatcccagacattcagcaggt |
| | | ccagctccagcagtctggccctgaactcgaaaaacctggcgctagcgtgaaaatttcctgtaaagcct |
| | | ccggctactcttttactggctacacaatgaattgggtgaaacagtctcacggcaaatccctcgaatgg |
| | | atcggactcatcacaccctacaatggcgcctcttcctacaaccagaaattccggggcaaggcaacact |
| | | cactgtggacaaatcatcctctaccgcctacatggatctgctctccctcacatctgaggactccgctg |
| | | tctacttttgtgcccgaggaggatacgacggacgaggattcgattactggggacagggaacaactgtg |
| | | accgtgtctagtggcggcggagggagtggaggcggaggatcttctggcgggggatccgatattgaa |
| | | ctcacacagtctcccgctatcatgtctgcttctcccggcgagaaagtgactatgacttgctctgcttc |
| | | ctcttctgtgtcctacatgcactggtaccagcagaaatctggcacatcccctaaacggtggatctacg |
| | | atactagcaaactggcatccggcgtgcctgggcgattctctggctctggctctggcaactcttactct |
| | | ctcacaatctcatctgtcgaggctgaggacgatgccacatactactgtcagcagtggtctaaacaccc |
| | | actcacattcggcgctggcactaaactggaaataaaagcggccgcaggtggcggcggttctggtggcg |
| | | gcggttctggtggcggcggttctctcgaggatggtaatgaagaaatgggtggtattacacagacacca |
| | | tataaagtctccatctctggaaccacagtaatattgacatgccctcagtatcctggatctgaaatact |
| | | atggcaacacaatgataaaaacataggcggtgatgaggatgataaaaacataggcagtgatgaggatc |
| | | acctgtcactgaaggaattttcagaattggagcaaagtggttattatgtctgctaccccagaggaagc |
| | | aaaccagaagatgcgaacttttatctctacctgagggcaagagtgtgtgagaactgcatggagatgga |
| | | tgtgatgtcggtggccacaattgtcatagtggacatctgcatcactgggggcttgctgctgctggttt |
| | | actactggagcaagaatagaaaggccaaggccaagcctgtgacacgaggagcgggtgctggcggca |
| | | ggcaaaggggacaaaacaaggagaggccaccacctgttcccaacccagactatgagcccatccg |
| | | gaaaggccagcgggacctgtattctggcctgaatcagagacgcatctgataagaattcgatccgcg |
| | | gccgcgaaggatctgcgatcgctccggtgcccgtcagtgggcagagcgcacatcgcccacagtcc |
| | | ccgagaagttggggggaggggtcggcaattgaacgggtgcctagagaaggtggcgcggggtaaa |
| | | ctgggaaagtgatgtcgtgtactggctccgcctttttcccgagggtgggggagaaccgtatataagtg |
| | | cagtagtcgccgtgaacgttctttttcgcaacgggtttgccgccagaacacagctgaagcttcgaggg |
| | | gctcgcatctctccttcacgcgcccgccgccctacctgaggccgccatccacgccggttgagtcgc |
| | | gttctgccgcctcccgcctgtggtgcctcctgaactgcgtccgccgtctaggtaagtttaaagctcag |
| | | gtcgagaccgggcctttgtccggcgctcccttggagcctacctagactcagccggctctccacgcttt |
| | | gcctgaccctgcttgctcaactctacgtctttgtttcgttttctgttctgcgccgttacagatccaag |
| | | ctgtgaccggcgcctacgctagatgaccgagtacaagcccacggtgcgcctcgccacccgcgacgacg |
| | | tccccagggccgtacgcaccctcgccgccgcgttcgccgactaccccgccacgcgccacaccgtc |
| | | gatccggaccgccacatcgagcgggtcaccgagctgcaagaactcttcctcacgcgcgtcgggct |
| | | cgacatcggcaaggtgtgggtcgcggacgacggcgccgcggtggcggtctggaccacgccgga |
| | | gagcgtcgaagcgggggcggtgttcgccgagatcggcccgcgcatggccgagttgagcggttcc |
| | | cggctggccgcgcagcaacagatggaaggcctcctggcgccgcaccggcccaaggagcccgcg |
| | | tggttcctggccaccgtcggcgtctcgcccgaccaccagggcaagggtctgggcagcgccgtcgt |
| | | gctccccggagtggaggcggccgagcgcgccggggtgcccgccttcctggagacctccgcgccc |
| | | cgcaacctccccttctacgagcggctcggcttcaccgtcaccgccgacgtcgaggtgcccgaagga |
| | | ccgcgcacctggtgcatgacccgcaagcccggtgcctgagtcgacaatcaacctctggattacaaa |
| | | atttgtgaaagattgactggtattcttaactatgttgctccttttacgctatgtggatacgctgcttt |
| | | aatgcctttgtatcatgctattgcttcccgtatggctttcattttctcctccttgtataaatcctggt |
| | | tgctgtctctttatgaggagttgtggcccgttgtcaggcaacgtggcgtggtgtgcactgtgtttgct |
| | | gacgcaacccccactggttggggcattgccaccacctgtcagctcctttccgggactttcgctttccc |
| | | cctccctattgccacggcggaactcatcgccgcctgccttgcccgctgctggacaggggctcggctgt |
| | | tgggcactgacaattccgtggtgttgtcggggaaatcatcgtcctttccttggctgctcgcctgtgtt |
| | | gccacctggattctgcgcgggacgtccttctgctacgtcccttcggccctcaatccagcggaccttcc |
| | | ttcccgcggcctgctgccggctctgcggcctcttccgcgtcttcgccttcgccctcagacgagtcgga |
| | | tctccctttgggccgcctccccgcctggtacctttaagaccaatgacttacaaggcagctgtagatct |
| | | tagccactttttaaaagaaaaggggggactggaagggctaattcactcccaacgaaaataagatctgc |
| | | tttttgcttgtactgggtctctctggttagaccagatctgagcctgggagctctctggctaactaggg |
| | | aacccactgcttaagcctcaataaagcttgccttgagtgcttcaagtagtgtgtgcccgtctgttgtg |
| | | tgactctggtaactagagatccctcagacccttttagtcagtgtggaaaatctctagcagtagtagtt |
| | | catgtcatcttattattcagtatttataacttgcaaagaaatgaatatcagagagtgagaggaacttg |
| | | tttattgcagcttataatggttacaaataaagcaatagcatcacaaatttcacaaataaagcattttt |
| | | ttcactgcattctagttgtggtttgtccaaactcatcaatgtatcttatcatgtctggctctagctat |
| | | cccgcccctaactccgcccagttccgcccattctccgccccatggctgactaattttttttatttatg |
| | | cagaggccgaggccgcctcggcctctgagctattccagaagtagtgaggaggcttttttggaggccta |
| | | gacttttgcagagacggcccaaattcgtaatcatggtcatagctgtttcctgtgtgaaattgttatcc |
| | | gctcacaattccacacaacatacgagccggaagcataaagtgtaaagcctggggtgcctaatgagtga |
| | | gctaactcacattaattgcgttgcgctcactgcccgctttccagtcgggaaacctgtcgtgccagctg |
| | | cattaatgaatcggccaacgcgcggggagaggcggtttgcgtattgggcgctcttccgcttcctcgct |
| | | cactgactcgctgcgctcggtcgttcggctgcggcgagcggtatcagctcactcaaaggcggtaatac |
| | | ggttatccacagaatcaggggataacgcaggaaagaacatgtgagcaaaaggccagcaaaaggccagg |
| | | aaccgtaaaaaggccgcgttgctggcgtttttccataggctccgcccccctgacgagcatcacaaaaa |
| | | tcgacgctcaagtcagaggtggcgaaacccgacaggactataaagataccaggcgtttccccctggaa |
| | | gctccctcgtgcgctctcctgttccgaccctgccgcttaccggatacctgtccgcctttctcccttcg |
| | | ggaagcgtggcgctttctcatagctcacgctgtaggtatctcagttcggtgtaggtcgttcgctccaa |
| | | gctgggctgtgtgcacgaaccccccgttcagcccgaccgctgcgccttatccggtaactatcgtcttg |
| | | agtccaacccggtaagacacgacttatcgccactggcagcagccactggtaacaggattagcagagcg |
| | | aggtatgtaggcggtgctacagagttcttgaagtggtggcctaactacggctacactagaaggacagt |
| | | atttggtatctgcgctctgctgaagccagttaccttcggaaaaagagttggtagctcttgatccggca |
| | | aacaaaccaccgctggtagcggtggtttttttgtttgcaagcagcagattacgcgcagaaaaaaagga |
| | | tctcaagaagatcctttgatcttttctacggggtctgacgctcagtggaacgaaaactcacgttaagg |
| | | gattttggtcatgagattatcaaaaaggatcttcacctagatccttttaaattaaaaatgaagtttta |
| | | aatcaatctaaagtatatatgagtaaacttggtctgacagttaccaatgcttaatcagtgaggcacct |
| | | atctcagcgatctgtctatttcgttcatccatagttgcctgactccccgtcgtgtagataactacgat |
| | | acgggagggcttaccatctggccccagtgctgcaatgataccgcgagacccacgctcaccggctccag |
| | | atttatcagcaataaaccagccagccggaagggccgagcgcagaagtggtcctgcaactttatccgcc |
| | | tccatccagtctattaattgttgccgggaagctagagtaagtagttcgccagttaatagtttgcgcaa |
| | | cgttgttgccattgctacaggcatcgtggtgtcacgctcgtcgtttggtatggcttcattcagctccg |
| | | gttcccaacgatcaaggcgagttacatgatcccccatgttgtgcaaaaaagcggttagctccttcggt |
| | | cctccgatcgttgtcagaagtaagttggccgcagtgttatcactcatggttatggcagcactgcataa |
| | | ttctcttactgtcatgccatccgtaagatgcttttctgtgactggtgagtactcaaccaagtcattct |
| | | gagaatagtgtatgcggcgaccgagttgctcttgcccggcgtcaatacgggataataccgcgccacat |
| | | agcagaactttaaaagtgctcatcattggaaaacgttcttcggggcgaaaactctcaaggatcttacc |
| | | gctgttgagatccagttcgatgtaacccactcgtgcacccaactgatcttcagcatcttttactttca |
| | | ccagcgtttctgggtgagcaaaaacaggaaggcaaaatgccgcaaaaaagggaataagggcgacacgg |
| | | aaatgttgaatactcatactcttcctttttcaatattattgaagcatttatcagggttattgtctcat |
| | | gagcggatacatatttgaatgtatttagaaaaataaacaaataggggttccgcgcacatttccccgaa |
| | | aagtgccacctgacgtctaagaaaccattattatcatgacattaacctataaaaataggcgtatcacg |
| | | aggccctttcgtctcgcgcgtttcggtgatgacggtgaaaacctctgacacatgcagctcccggagac |
| | | ggtcacagcttgtctgtaagcggatgccgggagcagacaagcccgtcagggcgcgtcagcgggtgttg |
| | | gcgggtgtcggggctggcttaactatgcggcatcagagcagattgtactgagagtgcaccatatgcgg |
| | | tgtgaaataccgcacagatgcgtaaggagaaaataccgcatcaggcgccattcgccattcaggctgcg |
| | | caactgttgggaagggcgatcggtgcgggcctcttcgctattacgccagctggcgaaagggggatgtg |
| | | ctgcaaggcgattaagttgggtaacgccagggttttcccagtcacgacgttgtaaaacgacggccagt |
| | | gccaagctg |
| 15 | MSLN amino | MALPTARPLLGSCGTPALGSLLFLLFSLGWVQPSRTLAGETGQE |
| | acid | AAPLDGVLANPPNISSLSPRQLLGFPCAEVSGLSTERVRELAVA |
| | sequence: | LAQKNVKLSTEQLRCLAHRLSEPPEDLDALPLDLLLFLNPDAFS |
| | human | GPQACTRFFSRITKANVDLLPRGAPERQRLLPAALACWGVRGS |
| | mesothelin | LLSEADVRALGGLACDLPGRFVAESAEVLLPRLVSCPGPLDQD |
| | sequence | QQEAARAALQGGGPPYGPPSTWSVSTMDALRGLLPVLGQPIIRS |
| | (UniProt | IPQGIVAAWRQRSSRDPSWRQPERTILRPRFRREVEKTACPSGK |
| | Accession | KAREIDESLIFYKKWELEACVDAALLATQMDRVNAIPFTYEQL |
| | No. | DVLKHKLDELYPQGYPESVIQHLGYLFLKMSPEDIRKWNVTSL |
| | Q13421) | ETLKALLEVNKGHEMSPQVATLIDRFVKGRGQLDKDTLDTLTA |
| | | FYPGYLCSLSPEELSSVPPSSIWAVRPQDLDTCDPRQLDVLYPK |
| | | ARLAFQNMNGSEYFVKIQSFLGGAPTEDLKALSQQNVSMDLAT |
| | | FMKLRTDAVLPLTVAEVQKLLGPHVEGLKAEERHRPVRDWILR |
| | | QRQDDLDTLGLGLQGGIPNGYLVLDLSMQEALSGTPCLLGPGP |
| | | VLTVLALLLASTLA |
| 16 | p510_anti- | acgcgtgtagtcttatgcaatactcttgtagtcttgcaacatggtaacgatgagttagcaacatgcct |
| | MSLN_SS1_C | tacaaggagagaaaaagcaccgtgcatgccgattggtggaagtaaggtggtacgatcgtgccttatta |
| | D3ϵ DNA | ggaaggcaacagacgggtctgacatggattggacgaaccactgaattgccgcattgcagagatattgt |
| | | atttaagtgcctagctcgatacaataaacgggtctctctggttagaccagatctgagcctgggagctc |
| | | tctggctaactagggaacccactgcttaagcctcaataaagcttgccttgagtgcttcaagtagtgtg |
| | | tgcccgtctgttgtgtgactctggtaactagagatccctcagacccttttagtcagtgtggaaaatct |
| | | ctagcagtggcgcccgaacagggacctgaaagcgaaagggaaaccagagctctctcgacgcaggactc |
| | | ggcttgctgaagcgcgcacggcaagaggcgaggggcggcgactggtgagtacgccaaaaattttg |
| | | actagcggaggctagaaggagagagatgggtgcgagagcgtcagtattaagcgggggagaatta |
| | | gatcgcgatgggaaaaaattcggttaaggccagggggaaagaaaaaatataaattaaaacatatagt |
| | | atgggcaagcagggagctagaacgattcgcagttaatcctggcctgttagaaacatcagaaggctgt |
| | | agacaaatactgggacagctacaaccatcccttcagacaggatcagaagaacttagatcattatataa |
| | | tacagtagcaaccctctattgtgtgcatcaaaggatagagataaaagacaccaaggaagctttagaca |
| | | agatagaggaagagcaaaacaaaagtaagaccaccgcacagcaagcggccactgatcttcagac |
| | | ctggaggaggagatatgagggacaattggagaagtgaattatataaatataaagtagtaaaaattgaa |
| | | ccattaggagtagcacccaccaaggcaaagagaagagtggtgcagagagaaaaaagagcagtgg |
| | | gaataggagctttgttccttgggttcttgggagcagcaggaagcactatgggcgcagcctcaatgac |
| | | gctgacggtacaggccagacaattattgtctggtatagtgcagcagcagaacaatttgctgagggcta |
| | | ttgaggcgcaacagcatctgttgcaactcacagtctggggcatcaagcagctccaggcaagaatcct |
| | | ggctgtggaaagatacctaaaggatcaacagctcctggggatttggggttgctctggaaaactcattt |
| | | gcaccactgctgtgccttggaatgctagttggagtaataaatctctggaacagattggaatcacacga |
| | | cctggatggagtgggacagagaaattaacaattacacaagcttaatacactccttaattgaagaatcg |
| | | caaaaccagcaagaaaagaatgaacaagaattattggaattagataaatgggcaagtttgtggaattg |
| | | gtttaacataacaaattggctgtggtatataaaattattcataatgatagtaggaggcttggtaggtt |
| | | taagaatagtttttgctgtactttctatagtgaatagagttaggcagggatattcaccattatcgttt |
| | | cagacccacctcccaaccccgaggggacccgacaggcccgaaggaatagaagaagaaggtggagagag |
| | | agacagagacagatccattcgattagtgaacggatctcgacggtatcggttaacttttaaaagaaaag |
| | | gggggattggggggtacagtgcaggggaaagaatagtagacataatagcaacagacatacaaacta |
| | | aagaattacaaaaacaaattacaaaattcaaaattttatcgatactagtattatgcccagtacatgac |
| | | cttatgggactttcctacttggcagtacatctacgtattagtcatcgctattaccatggtgatgcggt |
| | | tttggcagtacatcaatgggcgtggatagcggtttgactcacggggatttccaagtctccaccccatt |
| | | gacgtcaatgggagtttgttttggcaccaaaatcaacgggactttccaaaatgtcgtaacaactccgc |
| | | cccattgacgcaaatgggcggtaggcgtgtacggtgggaggtttatataagcagagctcgtttagtga |
| | | accgtcagatcgcctggagacgccatccacgctgttttgacctccatagaagattctagagccgccac |
| | | catgcttctcctggtgacaagccttctgctctgtgagttaccacacccagcattcctcctgatcccag |
| | | acattcagcaggtccagctccagcagtctggccctgaactcgaaaaacctggcgctagcgtgaaaatt |
| | | tcctgtaaagcctccggctactcttttactggctacacaatgaattgggtgaaacagtctcacggcaa |
| | | atccctcgaatggatcggactcatcacaccctacaatggcgcctcttcctacaaccagaaattccggg |
| | | gcaaggcaacactcactgtggacaaatcatcctctaccgcctacatggatctgctctccctcacatct |
| | | gaggactccgctgtctacttttgtgcccgaggaggatacgacggacgaggattcgattactggggaca |
| | | gggaacaactgtgaccgtgtctagtggcggcggagggagtggaggcggaggatcttctggcgggggat |
| | | ccgatattgaactcacacagtctcccgctatcatgtctgcttctcccggcgagaaagtgactatgact |
| | | tgctctgcttcctcttctgtgtcctacatgcactggtaccagcagaaatctggcacatcccctaaacg |
| | | gtggatctacgatactagcaaactggcatccggcgtgcctgggcgattctctggctctggctctggca |
| | | actcttactctctcacaatctcatctgtcgaggctgaggacgatgccacatactactgtcagcagtgg |
| | | tctaaacacccactcacattcggcgctggcactaaactggaaataaaagcggccgcaggtggcggcgg |
| | | ttctggtggcggcggttctggtggcggcggttctctcgaggatggtaatgaagaaatgggtggtatta |
| | | cacagacaccatataaagtctccatctctggaaccacagtaatattgacatgccctcagtatcctgga |
| | | tctgaaatactatggcaacacaatgataaaaacataggcggtgatgaggatgataaaaacataggcag |
| | | tgatgaggatcacctgtcactgaaggaattttcagaattggagcaaagtggttattatgtctgctacc |
| | | ccagaggaagcaaaccagaagatgcgaacttttatctctacctgagggcaagagtgtgtgagaactgc |
| | | atggagatggatgtgatgtcggtggccacaattgtcatagtggacatctgcatcactgggggcttgct |
| | | gctgctggtttactactggagcaagaatagaaaggccaaggccaagcctgtgacacgaggagcgggtg |
| | | ctggcggcaggcaaaggggacaaaacaaggagaggccaccacctgttcccaacccagactatgagccc |
| | | atccggaaaggccagcgggacctgtattctggcctgaatcagagacgcatctgataagaattcgatc |
| | | cgcggccgcgaaggatctgcgatcgctccggtgcccgtcagtgggcagagcgcacatcgcccac |
| | | agtccccgagaagttggggggaggggtcggcaattgaacgggtgcctagagaaggtggcgcggg |
| | | gtaaactgggaaagtgatgtcgtgtactggctccgcctttttcccgagggtgggggagaaccgtatat |
| | | aagtgcagtagtcgccgtgaacgttctttttcgcaacgggtttgccgccagaacacagctgaagcttc |
| | | gaggggctcgcatctctccttcacgcgcccgccgccctacctgaggccgccatccacgccggttga |
| | | gtcgcgttctgccgcctcccgcctgtggtgcctcctgaactgcgtccgccgtctaggtaagtttaaag |
| | | ctcaggtcgagaccgggcctttgtccggcgctcccttggagcctacctagactcagccggctctcca |
| | | cgctttgcctgaccctgcttgctcaactctacgtctttgtttcgttttctgttctgcgccgttacaga |
| | | tccaagctgtgaccggcgcctacgctagatgaccgagtacaagcccacggtgcgcctcgccacccgcg |
| | | acgacgtccccagggccgtacgcaccctcgccgccgcgttcgccgactaccccgccacgcgccaca |
| | | ccgtcgatccggaccgccacatcgagcgggtcaccgagctgcaagaactcttcctcacgcgcgtcg |
| | | ggctcgacatcggcaaggtgtgggtcgcggacgacggcgccgcggtggcggtctggaccacgcc |
| | | ggagagcgtcgaagcgggggcggtgttcgccgagatcggcccgcgcatggccgagttgagcggt |
| | | tcccggctggccgcgcagcaacagatggaaggcctcctggcgccgcaccggcccaaggagccc |
| | | gcgtggttcctggccaccgtcggcgtctcgcccgaccaccagggcaagggtctgggcagcgccgt |
| | | cgtgctccccggagtggaggcggccgagcgcgccggggtgcccgccttcctggagacctccgcg |
| | | ccccgcaacctccccttctacgagcggctcggcttcaccgtcaccgccgacgtcgaggtgcccgaa |
| | | ggaccgcgcacctggtgcatgacccgcaagcccggtgcctgagtcgacaatcaacctctggattac |
| | | aaaatttgtgaaagattgactggtattcttaactatgttgctccttttacgctatgtggatacgctg |
| | | ctttaatgcctttgtatcatgctattgcttcccgtatggctttcattttctcctccttgtataaatcc |
| | | tggttgctgtctctttatgaggagttgtggcccgttgtcaggcaacgtggcgtggtgtgcactgtgtt |
| | | tgctgacgcaacccccactggttggggcattgccaccacctgtcagctcctttccgggactttcgctt |
| | | tccccctccctattgccacggcggaactcatcgccgcctgccttgcccgctgctggacaggggctcgg |
| | | ctgttgggcactgacaattccgtggtgttgtcggggaaatcatcgtcctttccttggctgctcgcctg |
| | | tgttgccacctggattctgcgcgggacgtccttctgctacgtcccttcggccctcaatccagcggacc |
| | | ttccttcccgcggcctgctgccggctctgcggcctcttccgcgtcttcgccttcgccctcagacgagt |
| | | cggatctccctttgggccgcctccccgcctggtacctttaagaccaatgacttacaaggcagctgtag |
| | | atcttagccactttttaaaagaaaaggggggactggaagggctaattcactcccaacgaaaataagat |
| | | ctgctttttgcttgtactgggtctctctggttagaccagatctgagcctgggagctctctggctaact |
| | | agggaacccactgcttaagcctcaataaagcttgccttgagtgcttcaagtagtgtgtgcccgtctgt |
| | | tgtgtgactctggtaactagagatccctcagacccttttagtcagtgtggaaaatctctagcagtagt |
| | | agttcatgtcatcttattattcagtatttataacttgcaaagaaatgaatatcagagagtgagaggaa |
| | | cttgtttattgcagcttataatggttacaaataaagcaatagcatcacaaatttcacaaataaagcat |
| | | ttttttcactgcattctagttgtggtttgtccaaactcatcaatgtatcttatcatgtctggctctag |
| | | ctatcccgcccctaactccgcccagttccgcccattctccgccccatggctgactaattttttttatt |
| | | tatgcagaggccgaggccgcctcggcctctgagctattccagaagtagtgaggaggcttttttggagg |
| | | cctagacttttgcagagacggcccaaattcgtaatcatggtcatagctgtttcctgtgtgaaattgtt |
| | | atccgctcacaattccacacaacatacgagccggaagcataaagtgtaaagcctggggtgcctaatga |
| | | gtgagctaactcacattaattgcgttgcgctcactgcccgctttccagtcgggaaacctgtcgtgcca |
| | | gctgcattaatgaatcggccaacgcgcggggagaggcggtttgcgtattgggcgctcttccgcttcct |
| | | cgctcactgactcgctgcgctcggtcgttcggctgcggcgagcggtatcagctcactcaaaggcggta |
| | | atacggttatccacagaatcaggggataacgcaggaaagaacatgtgagcaaaaggccagcaaaaggc |
| | | caggaaccgtaaaaaggccgcgttgctggcgtttttccataggctccgcccccctgacgagcatcaca |
| | | aaaatcgacgctcaagtcagaggtggcgaaacccgacaggactataaagataccaggcgtttccccct |
| | | ggaagctccctcgtgcgctctcctgttccgaccctgccgcttaccggatacctgtccgcctttctccc |
| | | ttcgggaagcgtggcgctttctcatagctcacgctgtaggtatctcagttcggtgtaggtcgttcgct |
| | | ccaagctgggctgtgtgcacgaaccccccgttcagcccgaccgctgcgccttatccggtaactatcgt |
| | | cttgagtccaacccggtaagacacgacttatcgccactggcagcagccactggtaacaggattagcag |
| | | agcgaggtatgtaggcggtgctacagagttcttgaagtggtggcctaactacggctacactagaagga |
| | | cagtatttggtatctgcgctctgctgaagccagttaccttcggaaaaagagttggtagctcttgatcc |
| | | ggcaaacaaaccaccgctggtagcggtggtttttttgtttgcaagcagcagattacgcgcagaaaaaa |
| | | aggatctcaagaagatcctttgatcttttctacggggtctgacgctcagtggaacgaaaactcacgtt |
| | | aagggattttggtcatgagattatcaaaaaggatcttcacctagatccttttaaattaaaaatgaagt |
| | | tttaaatcaatctaaagtatatatgagtaaacttggtctgacagttaccaatgcttaatcagtgaggc |
| | | acctatctcagcgatctgtctatttcgttcatccatagttgcctgactccccgtcgtgtagataacta |
| | | cgatacgggagggcttaccatctggccccagtgctgcaatgataccgcgagacccacgctcaccggct |
| | | ccagatttatcagcaataaaccagccagccggaagggccgagcgcagaagtggtcctgcaactttatc |
| | | cgcctccatccagtctattaattgttgccgggaagctagagtaagtagttcgccagttaatagtttgc |
| | | gcaacgttgttgccattgctacaggcatcgtggtgtcacgctcgtcgtttggtatggcttcattcagc |
| | | tccggttcccaacgatcaaggcgagttacatgatcccccatgttgtgcaaaaaagcggttagctcctt |
| | | cggtcctccgatcgttgtcagaagtaagttggccgcagtgttatcactcatggttatggcagcactgc |
| | | ataattctcttactgtcatgccatccgtaagatgcttttctgtgactggtgagtactcaaccaagtca |
| | | ttctgagaatagtgtatgcggcgaccgagttgctcttgcccggcgtcaatacgggataataccgcgcc |
| | | acatagcagaactttaaaagtgctcatcattggaaaacgttcttcggggcgaaaactctcaaggatct |
| | | taccgctgttgagatccagttcgatgtaacccactcgtgcacccaactgatcttcagcatcttttact |
| | | ttcaccagcgtttctgggtgagcaaaaacaggaaggcaaaatgccgcaaaaaagggaataagggcgac |
| | | acggaaatgttgaatactcatactcttcctttttcaatattattgaagcatttatcagggttattgtc |
| | | tcatgagcggatacatatttgaatgtatttagaaaaataaacaaataggggttccgcgcacatttccc |
| | | cgaaaagtgccacctgacgtctaagaaaccattattatcatgacattaacctataaaaataggcgtat |
| | | cacgaggccctttcgtctcgcgcgtttcggtgatgacggtgaaaacctctgacacatgcagctcccgg |
| | | agacggtcacagcttgtctgtaagcggatgccgggagcagacaagcccgtcagggcgcgtcagcgggt |
| | | gttggcgggtgtcggggctggcttaactatgcggcatcagagcagattgtactgagagtgcaccatat |
| | | gcggtgtgaaataccgcacagatgcgtaaggagaaaataccgcatcaggcgccattcgccattcaggc |
| | | tgcgcaactgttgggaagggcgatcggtgcgggcctcttcgctattacgccagctggcgaaaggggga |
| | | tgtgctgcaaggcgattaagttgggtaacgccagggttttcccagtcacgacgttgtaaaacgacggc |
| | | cagtgccaagctg |
| 17 | p510_anti- | MLLLVTSLLLCELPHPAFLLIPDIQQVQLQQSGPELEKPGASVKI |
| | MSLN_SS1_C | SCKASGYSFTGYTMNWVKQSHGKSLEWIGLITPYNGASSYNQK |
| | D3ϵ amino | FRGKATLTVDKSSSTAYMDLLSLTSEDSAVYFCARGGYDGRGF |
| | acid | DYWGQGTTVTVSSGGGGSGGGGSSGGGSDIELTQSPAIMSASP |
| | | GEKVTMTCSASSSVSYMHWYQQKSGTSPKRWIYDTSKLASGV |
| | | PGRFSGSGSGNSYSLTISSVEAEDDATYYCQQWSKHPLTFGAGT |
| | | KLEIKAAAGGGGSGGGGSGGGGSLEDGNEEMGGITQTPYKVSI |
| | | SGTTVILTCPQYPGSEILWQHNDKNIGGDEDDKNIGSDEDHLSL |
| | | KEFSELEQSGYYVCYPRGSKPEDANFYLYLRARVCENCMEMD |
| | | VMSVATIVIVDICITGGLLLLVYYWSKNRKAKAKPVTRGAGAG |
| | | GRQRGQNKERPPPVPNPDYEPIRKGQRDLYSGLNQRRI* |
| 18 | Anti-MSLN | DVVMTQTPLSLPVSLGDQASISCRSSQSLVHSNGNTYLHWYLQ |
| | Light Chain | KPGQSPKLLIYKVSNRFSGVPDRFSGSGSGTDFTLKITRVEAEDL |
| | amino acid | GVFFCSQSTHVPFTFGSGTKLEIK |
| | (MHC1445LC. | |
| | 1) | |
| 19 | Anti-MSLN | gatgttgtgatgacccaaactccactctccctgcctgtcagtcttggagatcaagcctccatctcttg |
| | Light Chain | cagatctagtcagagccttgtacacagtaatggaaacacctatttacattggtacctgcagaagccag |
| | DNA | gccagtctccaaagctcctgatctacaaagtttccaaccgattttctggggtcccagacaggttcagt |
| | (MHC1445LC. | ggcagtggatcagggactgatttcacactcaagatcaccagagtggaggctgaggatctgggagtttt |
| | 1) | tttctgctctcaaagtacacatgttccattcacgttcggctcggggacaaagttggaaataaaa |
| 20 | Anti-MSLN | QVQLQQSGAELVRPGASVTLSCKASGYTFFDYEMHWVKQTPV |
| | Heavy Chain | HGLEWIGAIDPEIDGTAYNQKFKGKAILTADKSSSTAYMELRSL |
| | amino acid | TSEDSAVYYCTDYYGSSYWYFDVWGTGTTVTVSS |
| | (MHC1445HC. | |
| | 1) | |
| 21 | Anti-MSLN | caggttcaactgcagcagtctggggctgagctggtgaggcctggggcttcagtgacgctgtcctgca |
| | Heavy Chain | aggcttcgggctacacattttttgactatgaaatgcactgggtgaagcagacacctgtgcatggcctg |
| | DNA | gaatggattggagctattgatcctgaaattgatggtactgcctacaatcagaagttcaagggcaaggc |
| | (MHC1445HC. | catactgactgcagacaaatcctccagcacagcctacatggagctccgcagcctgacatctgagga |
| | 1) | ctctgccgtctattactgtacagattactacggtagtagctactggtacttcgatgtctggggcacag |
| | | ggaccacggtcaccgtctcctc |
| 22 | Anti-MSLN | DVMMTQTPLSLPVSLGDQASISCRSSQSLVHSNGNTYLHWFLQ |
| | Light Chain | KPGQSPKLLIYKVSNRFSGVPDRFSGSGSGTDFTLKISRVEAEDL |
| | amino acid | GVYFCSQTTHVPLTFGAGTKLELK |
| | (MHC1446LC. | |
| | 1) | |
| 23 | Anti-MSLN | gatgttatgatgacccaaactccactctccctgcctgtcagtcttggagatcaagcctccatctcttg |
| | Light Chain | cagatctagtcagagccttgtacacagtaatggaaacacctatttacattggttcctgcagaagccag |
| | DNA | gccagtctccaaagctcctgatctacaaagtttccaaccgattttctggggtcccagacaggttcagt |
| | (MHC1446LC. | ggcagtggatcagggacagatttcacactcaagatcagcagagtggaggctgaggatctgggagttta |
| | 1) | tttctgctctcaaactacacatgttccgctcacgttcggtgctgggaccaagctggagctgaaa |
| 24 | Anti-MSLN | QVQLQQSGAELVRPGASVTLSCKASGYTFTDYEMHWVKQTPV |
| | Heavy Chain | HGLEWIGAIDPEIAGTAYNQKFKGKAILTADKSSSTAYMELRSL |
| | amino acid | TSEDSAVYYCSRYGGNYLYYFDYWGQGTTLTVSS |
| | (MHC1446HC. | |
| | 3) | |
| 25 | Anti-MSLN | caggttcaactgcagcagtctggggctgagctggtgaggcctggggcttcagtgacgctgtcctgca |
| | Heavy Chain | aggcttcgggctacacttttactgactatgaaatgcactgggtgaagcagacacctgtccatggcctg |
| | DNA | gaatggattggagctattgatcctgaaattgctggtactgcctacaatcagaagttcaagggcaaggc |
| | (MHC1446HC. | catactgactgcagacaaatcctccagcacagcctacatggagctccgcagcctgacatctgagga |
| | 3) | ctctgccgtctattactgttcaagatacggtggtaactacctttactactttgactactggggccaag |
| | | gcaccactctcacagtctcctca |
| 26 | Anti-MSLN | DVLMTQIPLSLPVSLGDQASISCRSSQNIVYSNGNTYLEWYLQK |
| | Light Chain | PGQSPKLLIYKVSNRFSGVPDRFSGSGSGTDFTLKISRVEAEDLG |
| | amino acid | VYYCFQGSHVPFTFGSGTKLEIK |
| | (MHC1447LC. | |
| | 5) | |
| 27 | Anti-MSLN | gatgttttgatgacccaaattccactctccctgcctgtcagtcttggagatcaagcctccatctcttg |
| | Light Chain | cagatctagtcagaacattgtgtatagtaatggaaacacctatttagagtggtacctgcagaaaccag |
| | DNA | gccagtctccaaagctcctgatctacaaagtttccaaccgattttctggggtcccagacaggttcagt |
| | (MHC1447LC. | ggcagtggatcagggacagatttcacactcaagatcagcagagtggaggctgaggatctgggagttta |
| | 5) | ttactgctttcaaggttcacatgttccattcacgttcggctcggggacaaagttggaaataaaa |
| 28 | Anti-MSLN | QVQLQQSGAELVRPGASVTLSCKASGYTFTDYEMHWVKQTPV |
| | Heavy Chain | HGLEWIGAIDPEIGGSAYNQKFKGRAILTADKSSSTAYMELRSL |
| | amino acid | TSEDSAVYYCTGYDGYFWFAYWGQGTLVTVSS |
| | (MHC1447HC. | |
| | 5) | |
| 29 | Anti-MSLN | caggttcaactgcagcagtccggggctgagctggtgaggcctggggcttcagtgacgctgtcctgc |
| | Heavy Chain | aaggcttcgggctacacatttactgactatgaaatgcactgggtgaagcagacacctgtgcatggcct |
| | DNA | ggaatggattggagctattgatcctgaaattggtggttctgcctacaatcagaagttcaagggcaggg |
| | (MHC1447HC. | ccatattgactgcagacaaatcctccagcacagcctacatggagctccgcagcctgacatctgagga |
| | 5) | ctctgccgtctattattgtacgggctatgatggttacttttggtttgcttactggggccaagggactc |
| | | tggtcactgtctcttca |
| 30 | Anti-MSLN | ENVLTQSPAIMSASPGEKVTMTCSASSSVSYMHWYQQKSSTSP |
| | Light Chain | KLWIYDTSKLASGVPGRFSGSGSGNSYSLTISSMEAEDVATYYC |
| | amino acid | FQGSGYPLTFGSGTKLEIK |
| | (MHC1448LC. | |
| | 4) | |
| 31 | Anti-MSLN | gaaaatgttctcacccagtctccagcaatcatgtccgcatctccaggggaaaaggtcaccatgacctg |
| | Light Chain | cagtgctagctcaagtgtaagttacatgcactggtaccagcagaagtcaagcacctcccccaaactct |
| | DNA | ggatttatgacacatccaaactggcttctggagtcccaggtcgcttcagtggcagtgggtctggaaac |
| | (MHC1448LC. | tcttactctctcacgatcagcagcatggaggctgaagatgttgccacttattactgttttcaggggag |
| | 4) | tgggtacccactcacgttcggctcggggacaaagttggaaataaaa |
| 32 | Anti-MSLN | QVQLQQSGAELVRPGASVTLSCKASGYTFTDYEMHWVKQTPV |
| | Heavy Chain | HGLEWIGGIDPETGGTAYNQKFKGKAILTADKSSSTAYMELRS |
| | amino acid | LTSEDSAVYYCTSYYGSRVFWGTGTTVTVSS |
| | (MHC1448HC. | |
| | 3) | |
| 33 | Anti-MSLN | caggttcaactgcagcagtctggggctgagctggtgaggcctggggcttcagtgacgctgtcctgca |
| | Heavy Chain | aggcttcgggctacacatttactgactatgaaatgcactgggtgaaacagacacctgtgcatggcctg |
| | DNA | gaatggattggaggtattgatcctgaaactggtggtactgcctacaatcagaagttcaagggtaaggc |
| | (MHC1448HC. | catactgactgcagacaaatcctccagcacagcctacatggagctccgcagcctgacatctgagga |
| | 3) | ctctgccgtctattactgtacaagttactatggtagtagagtcttctggggcacagggaccacggtca |
| | | ccgtctcctca |
| 34 | Anti-MSLN | QIVLSQSPAILSAFPGEKVTMTCRASSSVSYMHWYQQKPGSSPK |
| | Light Chain | PWIYATSNLASGVPARFSGSGSGTSYSLTISSVEAEDAATYYCQ |
| | amino acid | QWSSNPPTLTFGAGTKLELK |
| | (MHC1449LC. | |
| | 3) | |
| 35 | Anti-MSLN | caaattgttctctcccagtctccagcaatcctgtctgcatttccaggggagaaggtcactatgacttg |
| | Light Chain | cagggccagctcaagtgtaagttacatgcactggtaccagcagaagccaggatcctcccccaaaccct |
| | DNA | ggatttatgccacatccaacctggcttctggagtccctgctcgcttcagtggcagtgggtctgggacc |
| | (MHC1449LC. | tcttactctctcacaatcagcagtgtggaggctgaagatgctgccacttattactgccagcagtggag |
| | 3) | tagtaacccacccacgctcacgttcggtgctgggaccaagctggagctgaaa |
| 36 | Anti-MSLN | QVQLQQSGAELARPGASVKLSCKASGYTFTSYGISWVKQRTGQ |
| | Heavy Chain | GLEWIGEIYPRSGNTYYNESFKGKVTLTADKSSGTAYMELRSLT |
| | amino acid | SEDSAVYFCARWGSYGSPPFYYGMDYWGQGTSVTVSS |
| | (MHC1449HC. | |
| | 3) | |
| 37 | Anti-MSLN | caggttcagctgcagcagtctggagctgagctggcgaggcctggggcttcagtgaagctgtcctgc |
| | Heavy Chain | aaggcttctggctacaccttcacaagctatggtataagctgggtgaagcagaggactggacagggc |
| | DNA | cttgagtggattggagagatttatcctagaagtggtaatacttactacaatgagagcttcaagggcaa |
| | (MHC1449HC. | ggtcacactgaccgcagacaaatcttccggcacagcgtacatggagctccgcagcctgacatctgag |
| | 3) | gactctgcggtctatttctgtgcaagatggggctcctacggtagtccccccttttactatggtatgga |
| | | ctactggggtcaaggaacctcagtcaccgtctcctca |
| 38 | Anti-MSLN | DVLMTQTPLSLPVSLGNQASISCRSSQSIVHSSGSTYLEWYLQK |
| | Light Chain | PGQSPKLLIYKVSNRFSGVPDRFSGSGSGTDFTLKISRVEAEDLG |
| | amino acid | VYYCFQGSHVPYTFGGGTKLEIK |
| | (MHC1450LC. | |
| | 3) | |
| 39 | Anti-MSLN | gatgttttgatgacccaaactccactctccctgcctgtcagtcttggaaatcaagcctccatctcttg |
| | Light Chain | cagatctagtcagagcattgtacatagtagtggaagcacctatttagaatggtacctgcagaaaccag |
| | DNA | gccagtctccaaagctcctgatctacaaagtttccaaccgattttctggggtcccagacaggttcagt |
| | (MHC1450LC. | ggcagtggatcagggacagatttcacactcaagatcagcagagtggaggctgaggatctgggagttta |
| | 3) | ttactgctttcaaggctcacatgttccatacacgttcggaggggggaccaagctggaaataaaa |
| 40 | Anti-MSLN | QVQLQQSGAELARPGTSVKVSCKASGYTFTSYGISWVKQRIGQ |
| | Heavy Chain | GLEWIGEIHPRSGNSYYNEKIRGKATLTADKSSSTAYMELRSLIS |
| | amino acid | EDSAVYFCARLITTVVANYYAMDYWGQGTSVTVSS |
| | (MHC1450HC. | |
| | 5) | |
| 41 | Anti-MSLN | caggttcagctgcagcagtctggagctgagctggcgaggcctgggacttcagtgaaggtgtcctgc |
| | Heavy Chain | aaggcttctggctataccttcacaagttatggtataagctgggtgaagcagagaattggacagggcct |
| | DNA | tgagtggattggagagattcatcctagaagtggtaatagttactataatgagaagatcaggggcaagg |
| | (MHC1450HC. | ccacactgactgcagacaaatcctccagcacagcgtacatggagctccgcagcctgatatctgagg |
| | 5) | actctgcggtctatttctgtgcaaggctgattactacggtagttgctaattactatgctatggactac |
| | | tggggtcaaggaacctcagtcaccgtctcctca |
| 42 | Anti-MSLN | DIVMSQSPSSLAVSAGEKVTMSCKSSQSLLNSRTRKNYLAWYQ |
| | Light Chain | QKPGQSPKLLIYWASTRESGVPDRFTGSGSGTDFTLTISSVQAE |
| | amino acid | DLAVYYCKQSYNLVTFGAGTKLELK |
| | (MHC1451LC. | |
| | 1) | |
| 43 | Anti-MSLN | gacattgtgatgtcacagtctccatcctccctggctgtgtcagcaggagagaaggtcactatgagctg |
| | Light Chain | caaatccagtcagagtctgctcaacagtagaacccgaaagaactacttggcttggtaccagcagaaa |
| | DNA | ccagggcagtctcctaaactgctgatctactgggcatccactagggaatctggggtccctgatcgctt |
| | (MHC1451LC. | cacaggcagtggatctgggacagatttcactctcaccatcagcagtgtgcaggctgaagacctggca |
| | 1) | gtttattactgcaaacaatcttataatctggtcacgttcggtgctgggaccaagctggagctgaaa |
| 44 | Anti-MSLN | QVQLQQSGAELVRPGASVTLSCKASGYTFFDYEMHWVKQTPV |
| | Heavy Chain | HGLEWIGAIDPEIDGTAYNQKFKGKAILTADKSSSTAYMELRSL |
| | amino acid | TSEDSAVYYCTDYYGSSYWYFDVWGTGTTVTVSS |
| | (MHC1451HC. | |
| | 2) | |
| 45 | Anti-MSLN | caggttcaactgcagcagtctggggctgagctggtgaggcctggggcttcagtgacgctgtcctgca |
| | Heavy Chain | aggcttcgggctacacattttttgactatgaaatgcactgggtgaagcagacacctgtgcatggcctg |
| | DNA | gaatggattggagctattgatcctgaaattgatggtactgcctacaatcagaagttcaagggcaaggc |
| | (MHC1451HC. | catactgactgcagacaaatcctccagcacagcctacatggagctccgcagcctgacatctgagga |
| | 2) | ctctgccgtctattactgtacagattactacggtagtagctactggtacttcgatgtctggggcacag |
| | | ggaccacggtcaccgtctcctc |
| 46 | Anti-MSLN | QIVLTQSPAIMSASPGEKVTISCSASSSVSYMYWYQQKPGSSPK |
| | Light Chain | PWIYRTSNLASGVPARFSGSGSGTSYSLTISSMEAEDAATYYCQ |
| | amino acid | QYHSYPLTFGAGTKLELK |
| | (MHC1452LC. | |
| | 1) | |
| 47 | Anti-MSLN | caaattgttctcacccagtctccagcaatcatgtctgcatctccaggggagaaggtcaccatatcctg |
| | Light Chain | cagtgccagctcaagtgtaagttacatgtactggtaccagcagaagccaggatcctcccccaaaccct |
| | DNA | ggatttatcgcacatccaacctggcttctggagtccctgctcgcttcagtggcagtgggtctgggacc |
| | (MHC1452LC. | tcttactctctcacaatcagcagcatggaggctgaagatgctgccacttattactgccagcagtatca |
| | 1) | tagttacccactcacgttcggtgctgggaccaagctggagctgaaa |
| 48 | Anti-MSLN | QIVLTQSPAIMSASPGERVTMTCSASSSVSSSYLYWYQQKSGSS |
| | Light Chain | PKLWIYSISNLASGVPARFSGSGSGTSYSLTINSMEAEDAATYY |
| | amino acid | CQQWSSNPQLTFGAGTKLELK |
| | (MHC1452LC. | |
| | 6) | |
| 49 | Anti-MSLN | caaattgttctcacccagtctccagcaatcatgtctgcatctcctggggaacgggtcaccatgacctg |
| | Light Chain | cagtgccagctcaagtgtaagttccagctacttgtactggtaccagcagaagtcaggatcctccccaa |
| | DNA | aactctggatttatagcatatccaacctggcttctggagtcccagctcgcttcagtggcagtgggtct |
| | (MHC1452LC. | gggacctcttactctctcacaatcaacagcatggaggctgaagatgctgccacttattactgccagca |
| | 6) | gtggagtagtaacccacagctcacgttcggtgctgggaccaagctggagctgaaa |
| | Anti-MSLN | QVQLKQSGAELVKPGASVKISCKASGYTFTDYYINWVKQRPGQ |
| | Heavy Chain | GLEWIGKIGPGSGSTYYNEKFKGKATLTADKSSSTAYMQLSSLT |
| | amino acid | SEDSAVYFCARTGYYVGYYAMDYWGQGTSVTVSS |
| | (MHC1452HC. | |
| | 2) | |
| 50 | Anti-MSLN | caggtccagctgaagcagtctggagctgagctggtgaagcctggggcttcagtgaagatatcctgc |
| | Heavy Chain | aaggcttctggctacaccttcactgactactatataaactgggtgaagcagaggcctggacagggcc |
| | DNA | ttgagtggattggaaagattggtcctggaagtggtagtacttactacaatgagaagttcaagggcaag |
| | (MHC1452HC. | gccacactgactgcagacaaatcctccagcacagcctacatgcagctcagcagcctgacatctgag |
| | 2) | gactctgcagtctatttctgtgcaagaactggttactacgttggttactatgctatggactactgggg |
| | | tcaaggaacctcagtcaccgtctcctca |
| 51 | Anti-MSLN | QVQLQQSGAELARPGASVKLSCKASGYTFTIYGISWVKQRTGQ |
| | Heavy Chain | GLEWIGEIYPRSDNTYYNEKFKGKATLTADKSSSTAYMELRSLT |
| | amino acid | SEDSAVYFCARWYSFYAMDYWGQGTSVTVSS |
| | (MHC1452HC. | |
| | 4) | |
| 52 | Anti-MSLN | caggttcagctgcagcagtctggagctgagctggcgaggcctggggcttcagtgaagctgtcctgc |
| | Heavy Chain | aaggcttctggctacaccttcacaatctatggtataagctgggtgaaacagagaactggacagggcc |
| | DNA | ttgagtggattggagagatttatcctagaagtgataatacttactacaatgagaagttcaagggcaag |
| | (MHC1452HC. | gccacactgactgcagacaaatcctccagcacagcgtacatggagctccgcagcctgacatctgagg |
| | 4) | actctgcggtctatttctgtgcaagatggtactcgttctatgctatggactactggggtcaaggaacc |
| | | tcagtcaccgtctcctca |
| 53 | Single | EVQLVESGGGLVQPGGSLRLSCAASGGDWSANFMYWYRQAP |
| | domain | GKQRELVARISGRGVVDYVESVKGRFTISRDNSKNTLYLQMNS |
| | anti-MSLN | LRAEDTAVYYCAVASYWGQGTLVTVSS |
| | binder | |
| | 1 (SD1) | |
| 54 | Single | EVQLVESGGGLVQPGGSLRLSCAASGSTSSINTMYWYRQAPGK |
| | domain | ERELVAFISSGGSTNVRDSVKGRFTISRDNSKNTLYLQMNSLRA |
| | anti-MSLN | EDTAVYYCNTYIPYGGTLHDFWGQGTLVTVSS |
| | binder | |
| | 4 (SD4) | |
| 55 | Single | QVQLVESGGGVVQAGGSLRLSCAASGSTFSIRAMRWYRQAPG |
| | domain | TERDLVAVIYGSSTYYADAVKGRFTISRDNSKNTLYLQMNSLR |
| | anti-MSLN | AEDTAVYYCNADTIGTARDYWGQGTLVTVSS |
| | binder | |
| | 6 (SD6) | |

What is claimed is:

1. A method of providing an anti-tumor immunity in a mammal comprising administering to the mammal an effective amount of a population of T cells transduced with a recombinant nucleic acid molecule encoding a T cell receptor (TCR) fusion protein (TFP) comprising
(a) a TCR subunit comprising
(i) at least a portion of a TCR extracellular domain, and
(ii) a TCR intracellular domain comprising a stimulatory domain from an intracellular signaling domain of CD3epsilon or CD3gamma; and
(b) a human or humanized antibody domain comprising an antigen binding domain that is an anti-mesothelin binding domain;
wherein the TCR subunit and the antibody domain are operatively linked,
wherein the TFP incorporates into a TCR when expressed in a T cell, and
wherein lower levels of cytokines are released following treatment compared to the cytokine levels of a mammal treated with a CAR-T cell comprising the same human or humanized antibody domain.

2. The method of claim 1, wherein the human or humanized antibody domain is the VHH domain set forth in SEQ ID NO:53, SEQ ID NO:54, or SEQ ID NO:55.

3. The method of claim 1, wherein the cell is an autologous T cell.

4. The method of claim 1, wherein the cell is an allogeneic T cell.

5. The method of any one of claims 1-4, wherein the mammal is a human.

6. A method of treating a mammal having a disease associated with expression of mesothelin comprising administering to the mammal an effective amount of a population of T cells transduced with a recombinant nucleic acid molecule encoding a T cell receptor (TCR) fusion protein (TFP) comprising
(a) a TCR subunit comprising
(i) at least a portion of a TCR extracellular domain, and
(ii) a TCR intracellular domain comprising a stimulatory domain from an intracellular signaling domain of CD3epsilon or CD3gamma; and
(b) a human or humanized antibody domain comprising an antigen binding domain that is an anti-mesothelin binding domain;
wherein the TCR subunit and the antibody domain are operatively linked,
wherein the TFP incorporates into a TCR when expressed in a T cell, and
wherein lower levels of cytokines are released following treatment compared to the cytokine levels of a mammal treated with a CAR-T cell comprising the same human or humanized antibody domain.

7. The method of claim 6, wherein the human or humanized antibody domain is the VHH domain set forth in SEQ ID NO:53, SEQ ID NO:54, or SEQ ID NO:55.

8. The method of claim 6, wherein the cell is an autologous T cell.

9. The method of claim 6, wherein the cell is an allogeneic T cell.

10. The method of claim 6, wherein the disease associated with mesothelin expression is selected from the group consisting of a proliferative disease, a cancer, a malignancy, and a non-cancer related indication associated with expression of mesothelin.

11. The method of claim 6, wherein the disease is a cancer selected from the group consisting of mesothelioma, renal cell carcinoma, stomach cancer, breast cancer, lung cancer, ovarian cancer, prostate cancer, colon cancer, cervical cancer, brain cancer, liver cancer, pancreatic cancer, thyroid cancer, bladder cancer, ureter cancer, kidney cancer, endometrial cancer, esophogeal cancer, gastric cancer, thymic carcinoma, cholangiocarcinoma and stomach cancer.

12. The method of claim 6, wherein the disease is a cancer selected from the group consisting of mesothelioma, papillary serous ovarian adenocarcinoma, clear cell ovarian carcinoma, mixed Mullerian ovarian carcinoma, endometroid mucinous ovarian carcinoma, pancreatic adenocarcinoma, ductal pancreatic adenocarcinoma, uterine serous carcinoma, lung adenocarcinoma, extrahepatic bile duct carcinoma, gastric adenocarcinoma, esophageal adenocarcinoma, colorectal adenocarcinoma, breast adenocarcinoma, a disease associated with mesothelin expression, and combinations thereof.

13. The method of claim 6, wherein the cells expressing a TFP molecule are administered in combination with an agent that increases the efficacy of a cell expressing a TFP molecule.

14. The method of claim 1, wherein for a given cytokine, at least 10% less amount of the given cytokine is released following treatment compared to an amount of the given cytokine of a mammal treated with a CAR-T cell comprising the same human or humanized antibody domain.

15. The method of claim 14, wherein the given cytokine comprises one or more cytokines selected from the group consisting of IL-2, IFN-γ, IL-4, TNF-α, IL-6, IL-13, IL-5, IL-10, sCD137, GM-CSF, MIP-1α, MIP-1β, and any combinations thereof.

16. The method of claim 1, wherein a tumor growth in the mammal is inhibited such that a size of the tumor is at most 10%, at most 20%, at most 30%, at most 40%, at most 50%, or at most 60% of a size of a tumor in a mammal treated with T cells that do not express the TFP after at least 8 days of treatment, wherein the mammal treated with T cells expressing TFP and the mammal treated with T cells that do not express the TFP have the same tumor size before the treatment.

17. The method of claim 16, wherein the tumor growth in the mammal is completely inhibited.

18. The method of claim 17, wherein the tumor growth in the mammal is completely inhibited for at least 20 days, at least 30 days, at least 40 days, at least 50 days, at least 60 days, at least 70 days, at least 80 days, at least 90 days, at least 100 days, or more.

19. The method of claim 1, wherein the population of T cells transduced with TFP kill similar amount of tumor cells compared to the CAR-T cells comprising the same human or humanized antibody domain.

20. The method of claim 1, wherein the population of T cells transduced with the TFP have a different gene expression profile than the CAR-T cells comprising the same human or humanized antibody domain.

21. The method of claim 20, wherein an expression level of a gene is different in the T cells transduced with the TFP than an expression level of the gene in the CAR-T cells comprising the same human or humanized antibody domain.

22. The method of claim 21, wherein the gene has a function in antigen presentation, TCR signaling, homeostasis, metabolism, chemokine signaling, cytokine signaling, toll like receptor signaling, MMP and adhesion molecule signaling, or TNFR related signaling.


SEQUENCE LISTING (73 sequences)

SEQ ID NO: 1 - protein, 17 aa, Artificial Sequence
  source: /note=“Description of Artificial Sequence Synthetic peptide”
GGGGSGGGGSGGGGSLE

SEQ ID NO: 2 - protein, 20 aa, Artificial Sequence
  source: /note=“Description of Artificial Sequence Synthetic peptide”
AAAGGGGSGGGGSGGGGSLE

SEQ ID NO: 3 - protein, 30 aa, Artificial Sequence
  source: /note=“Description of Artificial Sequence Synthetic polypeptide”
AAAIEVMYPPPYLGGGGSGGGGSGGGGSLE

SEQ ID NO: 4 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 5 - protein, 182 aa, Homo sapiens
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 6 - protein, 172 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNKS

SEQ ID NO: 7 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 8 - protein, 281 aa, Homo sapiens
MAGTWLLLLLALGCPALPTGVGGTPFPSLAPPIMLLVDGKQQMVVVCLVLDVAPPGLDSPIWFSAGNGSALDAFTYGPSPATDGTWTNLAHLSLPSEELASWEPLVCHTGPGAEGHSRSTQPMHLSGEASTARTCPQEPLRGTPGGALWLGVLRLLLFKLLLFDLLLTCSCLCDPAGPLPSPATTTRLRALGSHRLHPATETGGREATSSPRPQPRDRRWGDTPPGRKPGSPVWGEGSYLSSYPTCPAQAWCSRSALRAPSSSLGAFFAGDLPPPLQAGAA

SEQ ID NO: 9 - protein, 142 aa, Homo sapiens
PNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 10 - protein, 139 aa, Homo sapiens
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAAKGAGTASKLTFGTGTRLQVTL

SEQ ID NO: 11 - protein, 177 aa, Homo sapiens
EDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 12 - protein, 133 aa, Homo sapiens
MGTSLLCWMALCLLGADHADTGVSQNPRHNITKRGQNVTFRCDPISEHNRLYWYRQTLGQGPEFLTYFQNEAQLEKSRLLSDRFSAERPKGSFSTLEIQRTEQGDSAMYLCASSLAGLNQPQHFGDGTRLSIL

SEQ ID NO: 13 - protein, 135 aa, Homo sapiens
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSFSTCSANYGYTFGSGTRLTVV

SEQ ID NO: 14 - DNA, 8791 bp, Homo sapiens
acgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatactagtattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtttatataagcagagctcgtttagtgaaccgtcagatcgcctggagacgccatccacgctgttttgacctccatagaagattctagagccgccaccatgcttctcctggtgacaagccttctgctctgtgagttaccacacccagcattcctcctgatcccagacattcagcaggtccagctccagcagtctggccctgaactcgaaaaacctggcgctagcgtgaaaatttcctgtaaagcctccggctactcttttactggctacacaatgaattgggtgaaacagtctcacggcaaatccctcgaatggatcggactcatcacaccctacaatggcgcctcttcctacaaccagaaattccggggcaaggcaacactcactgtggacaaatcatcctctaccgcctacatggatctgctctccctcacatctgaggactccgctgtctacttttgtgcccgaggaggatacgacggacgaggattcgattactggggacagggaacaactgtgaccgtgtctagtggcggcggagggagtggaggcggaggatcttctggcgggggatccgatattgaactcacacagtctcccgctatcatgtctgcttctcccggcgagaaagtgactatgacttgctctgcttcctcttctgtgtcctacatgcactggtaccagcagaaatctggcacatcccctaaacggtggatctacgatactagcaaactggcatccggcgtgcctgggcgattctctggctctggctctggcaactcttactctctcacaatctcatctgtcgaggctgaggacgatgccacatactactgtcagcagtggtctaaacacccactcacattcggcgctggcactaaactggaaataaaagcggccgcaggtggcggcggttctggtggcggcggttctggtggcggcggttctctcgaggatggtaatgaagaaatgggtggtattacacagacaccatataaagtctccatctctggaaccacagtaatattgacatgccctcagtatcctggatctgaaatactatggcaacacaatgataaaaacataggcggtgatgaggatgataaaaacataggcagtgatgaggatcacctgtcactgaaggaattttcagaattggagcaaagtggttattatgtctgctaccccagaggaagcaaaccagaagatgcgaacttttatctctacctgagggcaagagtgtgtgagaactgcatggagatggatgtgatgtcggtggccacaattgtcatagtggacatctgcatcactgggggcttgctgctgctggtttactactggagcaagaatagaaaggccaaggccaagcctgtgacacgaggagcgggtgctggcggcaggcaaaggggacaaaacaaggagaggccaccacctgttcccaacccagactatgagcccatccggaaaggccagcgggacctgtattctggcctgaatcagagacgcatctgataagaattcgatccgcggccgcgaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaaaataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctagacttttgcagagacggcccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctg

SEQ ID NO: 15 - protein, 630 aa, Homo sapiens
MALPTARPLLGSCGTPALGSLLFLLFSLGWVQPSRTLAGETGQEAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQAPRRPLPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSGTPCLLGPGPVLTVLALLLASTLA

SEQ ID NO: 16 - DNA, 8791 bp, Artificial Sequence; identical to SEQ ID NO: 14
  source: /note=“Description of Artificial Sequence Synthetic polynucleotide”

SEQ ID NO: 17 - protein, 470 aa, Artificial Sequence
  source: /note=“Description of Artificial Sequence Synthetic polypeptide”
MLLLVTSLLLCELPHPAFLLIPDIQQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSGGGGSGGGGSSGGGSDIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGAGTKLEIKAAAGGGGSGGGGSGGGGSLEDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 18 - protein, 112 aa, Artificial Sequence
  source: /note=“Description of Artificial Sequence Synthetic polypeptide”
DVVMTQTPLSLPVSLGDQASISCRSSQSLVHSNGNTYLHWYLQKPGQSPKLLIYKVSNRFSGVPDRFSGSGSGTDFTLKITRVEAEDLGVFFCSQSTHVPFTFGSGTKLEIK

SEQ ID NO: 19 - DNA, 336 bp, Artificial Sequence
  source: /note=“Description of Artificial Sequence Synthetic polynucleotide”
gatgttgtgatgacccaaactccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggactgatttcacactcaagatcaccagagtggaggctgaggatctgggagtttttttctgctctcaaagtacacatgttccattcacgttcggctcggggacaaagttggaaataaaa

SEQ ID NO: 20 - protein, 120 aa, Artificial Sequence
  source: /note=“Description of Artificial Sequence Synthetic polypeptide”
QVQLQQSGAELVRPGASVTLSCKASGYTFFDYEMHWVKQTPVHGLEWIGAIDPEIDGTAYNQKFKGKAILTADKSSSTAYMELRSLTSEDSAVYYCTDYYGSSYWYFDVWGTGTTVTVSS

SEQ ID NO: 21 - DNA, 359 bp, Artificial Sequence
  source: /note=“Description of Artificial Sequence Synthetic polynucleotide”
caggttcaactgcagcagtctggggctgagctggtgaggcctggggcttcagtgacgctgtcctgcaaggcttcgggctacacattttttgactatgaaatgcactgggtgaagcagacacctgtgcatggcctggaatggattggagctattgatcctgaaattgatggtactgcctacaatcagaagttcaagggcaaggccatactgactgcagacaaatcctccagcacagcctacatggagctccgcagcctgacatctgaggactctgccgtctattactgtacagattactacggtagtagctactggtacttcgatgtctggggcacagggaccacggtcaccgtctcctc

SEQ ID NO: 22 - protein, 112 aa, Artificial Sequence
  source: /note=“Description of Artificial Sequence Synthetic polypeptide”
DVMMTQTPLSLPVSLGDQASISCRSSQSLVHSNGNTYLHWFLQKPGQSPKLLIYKVSNRFSGVPDRFSGSGSGTDFTLKISRVEAEDLGVYFCSQTTHVPLTFGAGTKLELK

SEQ ID NO: 23 - DNA, 336 bp, Artificial Sequence
  source: /note=“Description of Artificial Sequence Synthetic polynucleotide”
gatgttatgatgacccaaactccactctccctgcctgtcagtcttggagatcaagcctccatctcttgcagatctagtcagagccttgtacacagtaatggaaacacctatttacattggttcctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaactacacatgttccgctcacgttcggtgctgggaccaagctggagctgaaa

SEQ ID NO: 24 - protein, 120 aa, Artificial Sequence
  source: /note=“Description of Artificial Sequence Synthetic polypeptide”
QVQLQQSGAELVRPGASVTLSCKASGYTFTDYEMHWVKQTPVHGLEWIGAIDPEIAGTAYNQKFKGKAILTADKSSSTAYMELRSLTSEDSAVYYCSRYGGNYLYYFDYWGQGTTLTVSS

SEQ ID NO: 25 - DNA, 360 bp, Artificial Sequence
  source: /note=“Description of Artificial Sequence Synthetic polynucleotide”
caggttcaactgcagcagtctggggctgagctggtgaggcctggggcttcagtgacgctgtcctgcaaggcttcgggctacacttttactgactatgaaatgcactgggtgaagcagacacctgtccatggcctggaatggattggagctattgatcctgaaattgctggtactgcctacaatcagaagttcaagggcaaggccatactgactgcagacaaatcctccagcacagcctacatggagctccgcagcctgacatctgaggactctgccgtctattactgttcaagatacggtggtaactacctttactactttgactactggggccaaggcaccactctcacagtctcctca

SEQ ID NO: 26 - protein, 112 aa, Artificial Sequence
  source: /note=“Description of Artificial Sequence Synthetic polypeptide”
DVLMTQIPLSLPVSLGDQASISCRSSQNIVYSNGNTYLEWYLQKPGQSPKLLIYKVSNRFSGVPDRFSGSGSGTDFTLKISRVEAEDLGVYYCFQGSHVPFTFGSGTKLEIK

SEQ ID NO: 27 - DNA, 336 bp, Artificial Sequence
  source: /note=“Description of Artificial Sequence Synthetic polynucleotide”
gatgttttgatgacccaaattccactctccctgcctgtcagtcttggagatcaagcctccatctcttgcagatctagtcagaacattgtgtatagtaatggaaacacctatttagagtggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttccattcacgttcggctcggggacaaagttggaaataaaa

SEQ ID NO: 28 - protein, 118 aa, Artificial Sequence
  source: /note=“Description of Artificial Sequence Synthetic polypeptide”
QVQLQQSGAELVRPGASVTLSCKASGYTFTDYEMHWVKQTPVHGLEWIGAIDPEIGGSAYNQKFKGRAILTADKSSSTAYMELRSLTSEDSAVYYCTGYDGYFWFAYWGQGTLVTVSS

SEQ ID NO: 29 - DNA, 354 bp, Artificial Sequence
  source: /note=“Description of Artificial Sequence Synthetic polynucleotide”
caggttcaactgcagcagtccggggctgagctggtgaggcctggggcttcagtgacgctgtcctgcaaggcttcgggctacacatttactgactatgaaatgcactgggtgaagcagacacctgtgcatggcctggaatggattggagctattgatcctgaaattggtggttctgcctacaatcagaagttcaagggcagggccatattgactgcagacaaatcctccagcacagcctacatggagctccgcagcctgacatctgaggactctgccgtctattattgtacgggctatgatggttacttttggtttgcttactggggccaagggactctggtcactgtctcttca

SEQ ID NO: 30 - protein, 106 aa, Artificial Sequence
  source: /note=“Description of Artificial Sequence Synthetic polypeptide”
ENVLTQSPAIMSASPGEKVTMTCSASSSVSYMHWYQQKSSTSPKLWIYDTSKLASGVPGRFSGSGSGNSYSLTISSMEAEDVATYYCFQGSGYPLTFGSGTKLEIK

SEQ ID NO: 31 - DNA, 318 bp, Artificial Sequence
  source: /note=“Description of Artificial Sequence Synthetic polynucleotide”
gaaaatgttctcacccagtctccagcaatcatgtccgcatctccaggggaaaaggtcaccatgacctgcagtgctagctcaagtgtaagttacatgcactggtaccagcagaagtcaagcacctcccccaaactctggatttatgacacatccaaactggcttctggagtcccaggtcgcttcagtggcagtgggtctggaaactcttactctctcacgatcagcagcatggaggctgaagatgttgccacttattactgttttcaggggagtgggtacccactcacgttcggctcggggacaaagttggaaataaaa

SEQ ID NO: 32 - protein, 116 aa, Artificial Sequence
  source: /note=“Description of Artificial Sequence Synthetic polypeptide”
QVQLQQSGAELVRPGASVTLSCKASGYTFTDYEMHWVKQTPVHGLEWIGGIDPETGGTAYNQKFKGKAILTADKSSSTAYMELRSLTSEDSAVYYCTSYYGSRVFWGTGTTVTVSS

SEQ ID NO: 33 - DNA, 348 bp, Artificial Sequence
  source: /note=“Description of Artificial Sequence Synthetic polynucleotide”
caggttcaactgcagcagtctggggctgagctggtgaggcctggggcttcagtgacgctgtcctgcaaggcttcgggctacacatttactgactatgaaatgcactgggtgaaacagacacctgtgcatggcctggaatggattggaggtattgatcctgaaactggtggtactgcctacaatcagaagttcaagggtaaggccatactgactgcagacaaatcctccagcacagcctacatggagctccgcagcctgacatctgaggactctgccgtctattactgtacaagttactatggtagtagagtcttctggggcacagggaccacggtcaccgtctcctca

SEQ ID NO: 34 - protein, 108 aa, Artificial Sequence
  source: /note=“Description of Artificial Sequence Synthetic polypeptide”
QIVLSQSPAILSAFPGEKVTMTCRASSSVSYMHWYQQKPGSSPKPWIYATSNLASGVPARFSGSGSGTSYSLTISSVEAEDAATYYCQQWSSNPPTLTFGAGTKLELK

SEQ ID NO: 35 - DNA, 324 bp, Artificial Sequence
  source: /note=“Description of Artificial Sequence Synthetic polynucleotide”
caaattgttctctcccagtctccagcaatcctgtctgcatttccaggggagaaggtcactatgacttgcagggccagctcaagtgtaagttacatgcactggtaccagcagaagccaggatcctcccccaaaccctggatttatgccacatccaacctggcttctggagtccctgctcgcttcagtggcagtgggtctgggacctcttactctctcacaatcagcagtgtggaggctgaagatgctgccacttattactgccagcagtggagtagtaacccacccacgctcacgttcggtgctgggaccaagctggagctgaaa

SEQ ID NO: 36 - protein, 124 aa, Artificial Sequence
  source: /note=“Description of Artificial Sequence Synthetic polypeptide”
QVQLQQSGAELARPGASVKLSCKASGYTFTSYGISWVKQRTGQGLEWIGEIYPRSGNTYYNESFKGKVTLTADKSSGTAYMELRSLTSEDSAVYFCARWGSYGSPPFYYGMDYWGQGTSVTVSS

SEQ ID NO: 37 - DNA, 372 bp, Artificial Sequence
  source: /note=“Description of Artificial Sequence Synthetic polynucleotide”
caggttcagctgcagcagtctggagctgagctggcgaggcctggggcttcagtgaagctgtcctgcaaggcttctggctacaccttcacaagctatggtataagctgggtgaagcagaggactggacagggccttgagtggattggagagatttatcctagaagtggtaatacttactacaatgagagcttcaagggcaaggtcacactgaccgcagacaaatcttccggcacagcgtacatggagctccgcagcctgacatctgaggactctgcggtctatttctgtgcaagatggggctcctacggtagtccccccttttactatggtatggactactggggtcaaggaacctcagtcaccgtctcctca

SEQ ID NO: 38 - protein, 112 aa, Artificial Sequence
  source: /note=“Description of Artificial Sequence Synthetic polypeptide”
DVLMTQTPLSLPVSLGNQASISCRSSQSIVHSSGSTYLEWYLQKPGQSPKLLIYKVSNRFSGVPDRFSGSGSGTDFTLKISRVEAEDLGVYYCFQGSHVPYTFGGGTKLEIK

SEQ ID NO: 39 - DNA, 336 bp, Artificial Sequence
  source: /note=“Description of Artificial Sequence Synthetic polynucleotide”
gatgttttgatgacccaaactccactctccctgcctgtcagtcttggaaatcaagcctccatctcttgcagatctagtcagagcattgtacatagtagtggaagcacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggctcacatgttccatacacgttcggaggggggaccaagctggaaataaaa

SEQ ID NO: 40 - protein, 123 aa, Artificial Sequence
  source: /note=“Description of Artificial Sequence Synthetic polypeptide”
QVQLQQSGAELARPGTSVKVSCKASGYTFTSYGISWVKQRIGQGLEWIGEIHPRSGNSYYNEKIRGKATLTADKSSSTAYMELRSLISEDSAVYFCARLITTVVANYYAMDYWGQGTSVTVSS

SEQ ID NO: 41 - DNA, 369 bp, Artificial Sequence
  source: /note=“Description of Artificial Sequence Synthetic polynucleotide”
caggttcagctgcagcagtctggagctgagctggcgaggcctgggacttcagtgaaggtgtcctgcaaggcttctggctataccttcacaagttatggtataagctgggtgaagcagagaattggacagggccttgagtggattggagagattcatcctagaagtggtaatagttactataatgagaagatcaggggcaaggccacactgactgcagacaaatcctccagcacagcgtacatggagctccgcagcctgatatctgaggactctgcggtctatttctgtgcaaggctgattactacggtagttgctaattactatgctatggactactggggtcaaggaacctcagtcaccgtctcctca

SEQ ID NO: 42 - protein, 112 aa, Artificial Sequence
  source: /note=“Description of Artificial Sequence Synthetic polypeptide”
DIVMSQSPSSLAVSAGEKVTMSCKSSQSLLNSRTRKNYLAWYQQKPGQSPKLLIYWASTRESGVPDRFTGSGSGTDFTLTISSVQAEDLAVYYCKQSYNLVTFGAGTKLELK

SEQ ID NO: 43 - DNA, 336 bp, Artificial Sequence
  source: /note=“Description of Artificial Sequence Synthetic polynucleotide”
gacattgtgatgtcacagtctccatcctccctggctgtgtcagcaggagagaaggtcactatgagctgcaaatccagtcagagtctgctcaacagtagaacccgaaagaactacttggcttggtaccagcagaaaccagggcagtctcctaaactgctgatctactgggcatccactagggaatctggggtccctgatcgcttcacaggcagtggatctgggacagatttcactctcaccatcagcagtgtgcaggctgaagacctggcagtttattactgcaaacaatcttataatctggtcacgttcggtgctgggaccaagctggagctgaaa

SEQ ID NO: 44 - protein, 120 aa, Artificial Sequence; identical to SEQ ID NO: 20
  source: /note=“Description of Artificial Sequence Synthetic polypeptide”

SEQ ID NO: 45 - DNA, 359 bp, Artificial Sequence; identical to SEQ ID NO: 21
  source: /note=“Description of Artificial Sequence Synthetic polynucleotide”

SEQ ID NO: 46 - protein, 106 aa, Artificial Sequence
  source: /note=“Description of Artificial Sequence Synthetic polypeptide”
QIVLTQSPAIMSASPGEKVTISCSASSSVSYMYWYQQKPGSSPKPWIYRTSNLASGVPARFSGSGSGTSYSLTISSMEAEDAATYYCQQYHSYPLTFGAGTKLELK

SEQ ID NO: 47 - DNA, 318 bp, Artificial Sequence
  source: /note=“Description of Artificial Sequence Synthetic polynucleotide”
caaattgttctcacccagtctccagcaatcatgtctgcatctccaggggagaaggtcaccatatcctgcagtgccagctcaagtgtaagttacatgtactggtaccagcagaagccaggatcctcccccaaaccctggatttatcgcacatccaacctggcttctggagtccctgctcgcttcagtggcagtgggtctgggacctcttactctctcacaatcagcagcatggaggctgaagatgctgccacttattactgccagcagtatcatagttacccactcacgttcggtgctgggaccaagctggagctgaaa

SEQ ID NO: 48 - protein, 109 aa, Artificial Sequence
  source: /note=“Description of Artificial Sequence Synthetic polypeptide”
QIVLTQSPAIMSASPGERVTMTCSASSSVSSSYLYWYQQKSGSSPKLWIYSISNLASGVPARFSGSGSGTSYSLTINSMEAEDAATYYCQQWSSNPQLTFGAGTKLELK

SEQ ID NO: 49 - DNA, 327 bp, Artificial Sequence
  source: /note=“Description of Artificial Sequence Synthetic polynucleotide”
caaattgttctcacccagtctccagcaatcatgtctgcatctcctggggaacgggtcaccatgacctgcagtgccagctcaagtgtaagttccagctacttgtactggtaccagcagaagtcaggatcctccccaaaactctggatttatagcatatccaacctggcttctggagtcccagctcgcttcagtggcagtgggtctgggacctcttactctctcacaatcaacagcatggaggctgaagatgctgccacttattactgccagcagtggagtagtaacccacagctcacgttcggtgctgggaccaagctggagctgaaa

SEQ ID NO: 50 - DNA, 363 bp, Artificial Sequence
  source: /note=“Description of Artificial Sequence Synthetic polynucleotide”
caggtccagctgaagcagtctggagctgagctggtgaagcctggggcttcagtgaagatatcctgcaaggcttctggctacaccttcactgactactatataaactgggtgaagcagaggcctggacagggccttgagtggattggaaagattggtcctggaagtggtagtacttactacaatgagaagttcaagggcaaggccacactgactgcagacaaatcctccagcacagcctacatgcagctcagcagcctgacatctgaggactctgcagtctatttctgtgcaagaactggttactacgttggttactatgctatggactactggggtcaaggaacctcagtcaccgtctcctca

SEQ ID NO: 51 - protein, 118 aa, Artificial Sequence
  source: /note=“Description of Artificial Sequence Synthetic polypeptide”
QVQLQQSGAELARPGASVKLSCKASGYTFTIYGISWVKQRTGQGLEWIGEIYPRSDNTYYNEKFKGKATLTADKSSSTAYMELRSLTSEDSAVYFCARWYSFYAMDYWGQGTSVTVSS

SEQ ID NO: 52 - DNA, 354 bp, Artificial Sequence
  source: /note=“Description of Artificial Sequence Synthetic polynucleotide”
caggttcagctgcagcagtctggagctgagctggcgaggcctggggcttcagtgaagctgtcctgcaaggcttctggctacaccttcacaatctatggtataagctgggtgaaacagagaactggacagggccttgagtggattggagagatttatcctagaagtgataatacttactacaatgagaagttcaagggcaaggccacactgactgcagacaaatcctccagcacagcgtacatggagctccgcagcctgacatctgaggactctgcggtctatttctgtgcaagatggtactcgttctatgctatggactactggggtcaaggaacctcagtcaccgtctcctca

SEQ ID NO: 53 - protein, 111 aa, Artificial Sequence
  source: /note=“Description of Artificial Sequence Synthetic polypeptide”
EVQLVESGGGLVQPGGSLRLSCAASGGDWSANFMYWYRQAPGKQRELVARISGRGVVDYVESVKGRFTISRDNSKNTLYLQMNSLRAEDTAVYYCAVASYWGQGTLVTVSS

SEQ ID NO: 54 - protein, 119 aa, Artificial Sequence
  source: /note=“Description of Artificial Sequence Synthetic polypeptide”
EVQLVESGGGLVQPGGSLRLSCAASGSTSSINTMYWYRQAPGKERELVAFISSGGSTNVRDSVKGRFTISRDNSKNTLYLQMNSLRAEDTAVYYCNTYIPYGGTLHDFWGQGTLVTVSS

SEQ ID NO: 55 - protein, 116 aa, Artificial Sequence
  source: /note=“Description of Artificial Sequence Synthetic polypeptide”
QVQLVESGGGVVQAGGSLRLSCAASGSTFSIRAMRWYRQAPGTERDLVAVIYGSSTYYADAVKGRFTISRDNSKNTLYLQMNSLRAEDTAVYYCNADTIGTARDYWGQGTLVTVSS

SEQ ID NO: 56 - protein, 10 aa, Artificial Sequence
  source: /note=“Description of Artificial Sequence Synthetic peptide”
GGGGSGGGGS

SEQ ID NO: 57 - DNA, 30 bp, Artificial Sequence
  source: /note=“Description of Artificial Sequence Synthetic oligonucleotide”
ggtggcggaggttctggaggtggaggttcc

SEQ ID NO: 58 - protein, 280 aa, Homo sapiens
MAGTWLLLLLALGCPALPTGVGGTPFPSLAPPIMLLVDGKQQMVVVCLVLDVAPPGLDSPIWFSAGNGSALDAFTYGPSPATDGTWTNLAHLSLPSEELASWEPLVCHTGPGAEGHSRSTQPMHLSGEASTARTCPQEPLRGTPGGALWLGVLRLLLFKLLLFDLLLTCSCLCDPAGPLPSPATTTRLRALGSHRLHPATETGGREATSSPRPQPRDRRWGDTPPGRKPGSPVWGEGSYLSSYPTCPAQAWCSRSALRAPSSSLGAFFAGDLPPPLQAGA

SEQ ID NO: 59 - protein, 622 aa, Homo sapiens
MALPTARPLLGSCGTPALGSLLFLLFSLGWVQPSRTLAGETGQEAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSGTPCLLGPGPVLTVLALLLASTLA

SEQ ID NO: 60 - protein, 20 aa, Artificial Sequence
  source: /note=“Description of Artificial Sequence Synthetic peptide”
  SITE (1)..(20): /note=“This sequence may encompass 1-4 ′Gly Gly Gly Gly Ser′ repeating units”
GGGGSGGGGSGGGGSGGGGS

SEQ ID NO: 61 - protein, 20 aa, Artificial Sequence
  source: /note=“Description of Artificial Sequence Synthetic peptide”
  SITE (1)..(20): /note=“This sequence may encompass 2-4 ′Gly Gly Gly Gly Ser′ repeating units”
GGGGSGGGGSGGGGSGGGGS

SEQ ID NO: 62 - protein, 15 aa, Artificial Sequence
  source: /note=“Description of Artificial Sequence Synthetic peptide”
  SITE (1)..(15): /note=“This sequence may encompass 1-3 ′Gly Gly Gly Gly Ser′ repeating units”
GGGGSGGGGSGGGGS

SEQ ID NO: 63 - protein, 121 aa, Artificial Sequence
  source: /note=“Description of Artificial Sequence Synthetic polypeptide”
QVQLKQSGAELVKPGASVKISCKASGYTFTDYYINWVKQRPGQGLEWIGKIGPGSGSTYYNEKFKGKATLTADKSSSTAYMQLSSLTSEDSAVYFCARTGYYVGYYAMDYWGQGTSVTVSS

SEQ ID NO: 64 - protein, 20 aa, Artificial Sequence
  source: /note=“Description of Artificial Sequence Synthetic peptide”
GGGGSGGGGSGGGGSGGGGS

SEQ ID NO: 65 - protein, 15 aa, Artificial Sequence
  source: /note=“Description of Artificial Sequence Synthetic peptide”
GGGGSGGGGSGGGGS

SEQ ID NO: 66 - protein, 4 aa, Artificial Sequence
  source: /note=“Description of Artificial Sequence Synthetic peptide”
GGGS

SEQ ID NO: 67 - DNA, 5000 bp, Artificial Sequence
  source: /note=“Description of Artificial Sequence Synthetic polynucleotide”
  MISC_FEATURE (1)..(5000): /note=“This sequence may encompass 50-5000 nucleotides”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 68 - protein, 30 aa, Artificial Sequence
  source: /note=“Description of Artificial Sequence Synthetic polypeptide”
  SITE (1)..(30): /note=“This sequence may encompass 1-6 ′Gly Gly Gly Gly Ser′ repeating units”
GGGGSGGGGSGGGGSGGGGSGGGGSGGGGS

SEQ ID NO: 69 - protein, 5 aa, Artificial Sequence
  source: /note=“Description of Artificial Sequence Synthetic peptide”
GGGGS

SEQ ID NO: 70 - DNA, 100 bp, Artificial Sequence
  source: /note=“Description of Artificial Sequence Synthetic polynucleotide”
tttttttttttttttttttttttttttttttttttttttttttttttttttttttttttttttttttttttttttttttttttttttttttttttttttt

SEQ ID NO: 71 - DNA, 5000 bp, Artificial Sequence
  source: /note=“Description of Artificial Sequence Synthetic polynucleotide”
  MISC_FEATURE (1)..(5000): /note=“This sequence may encompass 50-5000 nucleotides”
tttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttt

SEQ ID NO: 72 - DNA, 5000 bp, Artificial Sequence; identical to SEQ ID NO: 67
  source: /note=“Description of Artificial Sequence Synthetic polynucleotide”
  MISC_FEATURE (1)..(5000): /note=“This sequence may encompass 100-5000 nucleotides”

SEQ ID NO: 73 - DNA, 400 bp, Artificial Sequence
  source: /note=“Description of Artificial Sequence Synthetic polynucleotide”
  MISC_FEATURE (1)..(5000): /note=“This sequence may encompass 100-400 nucleotides”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa